SEQUENCE LISTING (260 sequences)

SEQ ID NO: 1 - DNA, 18 bp, Artificial sequence
  Synthetic
cttggtggtcctggctgc

SEQ ID NO: 2 - DNA, 23 bp, Artificial sequence
  Synthetic
ggtacgtgctgttgaactgttcc

SEQ ID NO: 3 - DNA, 47 bp, Artificial sequence
  Synthetic
catgccatgactgtggcccaggcggcccagktgcagctcgtggagtc

SEQ ID NO: 4 - DNA, 51 bp, Artificial sequence
  Synthetic
catgccatgactcgcggccggcctggccatgggggtcttcgctgtggtgcg

SEQ ID NO: 5 - DNA, 52 bp, Artificial sequence
  Synthetic
catgccatgactcgcggccggcctggccgtcttgtggttttggtgtcttggg

SEQ ID NO: 6 - protein, 217 aa, Artificial sequence
  Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 15 aa, Artificial sequence
  Synthetic
EPKSADKTHTCPPCP

SEQ ID NO: 8 - protein, 119 aa, Artificial sequence
  Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 9 - protein, 107 aa, Artificial sequence
  Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 10 - protein, 124 aa, Artificial sequence
  Synthetic
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 11 - protein, 118 aa, Artificial sequence
  Synthetic
EVQLVESGGGLVKPGGSLRLSCEASGFTFSDAWMSWVRQAPGKGLEWVGRIKSKTDGGTTDYVVPLNGRFIISRDDSRNTLYLQLNNLKTEDTAVYYCTTVPGSYGYWGQGTLVTVSS

SEQ ID NO: 12 - protein, 112 aa, Artificial sequence
  Synthetic
DIVMTQSPLSLPVTPGEPASISCRSSQSLLHNKRNNYLDWFLQKPGQSPQLLIYLASNRASGVPDRFSGGGSGTDFTLKISRVEAEDVGVYYCMQAQQTPITFGQGTRLEIK

SEQ ID NO: 13 - protein, 120 aa, Artificial sequence [sequence omitted]

SEQ ID NO: 14 - protein, 107 aa, Artificial sequence [sequence omitted]

SEQ ID NO: 15 - protein, 943 aa, Artificial sequence
  Synthetic
MRPSGTAGAALLALLAALCPASRALEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 16 - protein, 354 aa, Artificial sequence
  Synthetic
LEEKKGNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 17 - protein, 1210 aa, Artificial sequence [sequence omitted]

SEQ ID NO: 18 - protein, 1210 aa, Artificial sequence
  Synthetic
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSEFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDTLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSSQKTKIISNRGENSCKATGQVCHALCSPEGCWGPEPRDCVSCQNVSRGRECVDKCNILEGEPREFVENSECIQCHPECLPQVMNITCTGRGPDNCIQCAHYIDGPHCVKTCPAGVMGENNTLVWKYADAGHVCHLCHPNCTYGCTGPGLEGCARNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 19 - protein, 114 aa, Artificial sequence
  Synthetic
QVQLVESGGGLVQAGGSLRLSCAAFISTFGRTDMTWYRQRSGNEREFVARISSGGSTIYADSAKGRFTISRDNVKNTVYLQMNSLTPEDTAVYYCNTVPPRGSWSQGTQVTVSS

SEQ ID NO: 20 - DNA, 342 bp, Artificial sequence
  Synthetic
caggtgcagctggtggagagcggcggcggcctggtgcaggccggcggcagcctgaggctgagctgcgccgccttcatcagcaccttcggcaggaccgacatgacctggtacaggcagaggagcggcaacgagagggagttcgtggccaggatcagcagcggcggcagcaccatctacgccgacagcgccaagggcaggttcaccatcagcagggacaacgtgaagaacaccgtgtacctgcagatgaacagcctgacccccgaggacaccgccgtgtactactgcaacaccgtgccccccaggggcagctggagccagggcacccaggtgaccgtgagcagc

SEQ ID NO: 21 - protein, 117 aa, Artificial sequence
  Synthetic
EVQVVESGGGLVQPGGSLRLSCAASDDISSIYTMAWYRQAPGKQRELVALVTPGGGTNYADSVKGRFTISRDNAKNTVYLQMNSLKPEDTAVYYCNARHRVTGFAYWGQGTQVTVSS

SEQ ID NO: 22 - DNA, 351 bp, Artificial sequence
  Synthetic
gaggtgcaggtggtggagagcggcggcggcctggtgcagcccggcggcagcctgaggctgagctgcgccgccagcgacgacatcagcagcatctacaccatggcctggtacaggcaggcccccggcaagcagagggagctggtggccctggtgacccccggcggcggcaccaactacgccgacagcgtgaagggcaggttcaccatcagcagggacaacgccaagaacaccgtgtacctgcagatgaacagcctgaagcccgaggacaccgccgtgtactactgcaacgccaggcacagggtgaccggcttcgcctactggggccagggcacccaggtgaccgtgagcagc

SEQ ID NO: 23 - protein, 120 aa, Artificial sequence
  Synthetic
EVQVVESGGGLVQAGGSLRLSCAASGSTFSSYAMTWYRQAPGKQRELVAAISSGGSTNYAASVKGRFTISRDNAKNTLYLQMNTLKPEDTAVYYCNTDWGNGFSAEYDYWGQGTQVTVSS

SEQ ID NO: 24 - DNA, 360 bp, Artificial sequence
  Synthetic
gaggtgcaggtggtggagagcggcggcggcctggtgcaggccggcggcagcctgaggctgagctgcgccgccagcggcagcaccttcagcagctacgccatgacctggtacaggcaggcccccggcaagcagagggagctggtggccgccatcagcagcggcggcagcaccaactacgccgccagcgtgaagggcaggttcaccatcagcagggacaacgccaagaacaccctgtacctgcagatgaacaccctgaagcccgaggacaccgccgtgtactactgcaacaccgactggggcaacggcttcagcgccgagtacgactactggggccagggcacccaggtgaccgtgagcagc

SEQ ID NO: 25 - protein, 114 aa, Artificial sequence
  Synthetic
EVQLVESGGGLVQPGGSLRLSCAVSQSISSINAMDWYRQAPGKQRELVAIIFNNGRTNYADSVKGRFTISRDNARNTVYLQMNSLKPEDTAVYYCNIVPPLRNWGQGTQVTVSS

SEQ ID NO: 26 - DNA, 342 bp, Artificial sequence
  Synthetic
gaggtgcagctggtggagagcggcggcggcctggtgcagcccggcggcagcctgaggctgagctgcgccgtgagccagagcatcagcagcatcaacgccatggactggtacaggcaggcccccggcaagcagagggagctggtggccatcatcttcaacaacggcaggaccaactacgccgacagcgtgaagggcaggttcaccatcagcagggacaacgccaggaacaccgtgtacctgcagatgaacagcctgaagcccgaggacaccgccgtgtactactgcaacatcgtgccccccctgaggaactggggccagggcacccaggtgaccgtgagcagc

SEQ ID NO: 27 - protein, 116 aa, Artificial sequence
  Synthetic
EVQLVESGGGLVRAGGSLRLSCAASGTISSYDVVGWYRQAPGKQRELVALIGTDRWLNLGDFAKGRFTMSTDDAANTVDLEMNSLKPEDTAVYYCYTFQHTVGPRWGQGTQVTVSS

SEQ ID NO: 28 - DNA, 348 bp, Artificial sequence
  Synthetic
gaggtgcagctggtggagagcggcggcggcctggtgagggccggcggcagcctgaggctgagctgcgccgccagcggcaccatcagcagctacgacgtggtgggctggtacaggcaggcccccggcaagcagagggagctggtggccctgatcggcaccgacaggtggctgaacctgggcgacttcgccaagggcaggttcaccatgagcaccgacgacgccgccaacaccgtggacctggagatgaacagcctgaagcccgaggacaccgccgtgtactactgctacaccttccagcacaccgtgggccccaggtggggccagggcacccaggtgaccgtgagcagc

SEQ ID NO: 29 - protein, 114 aa, Artificial sequence
  Synthetic
QLQLVESGGGLVQAGGSLKLSCVASGFTFSNYAMAWYRQAPGKQRELIASCSASCIWTNYGASVKGRFTMSLDNAKKTVYLQMDSLKPEDTAVYYCRNLDANYWGQGTQVTVSS

SEQ ID NO: 30 - DNA, 342 bp, Artificial sequence
  Synthetic
cagctgcagctggtggagagcggcggcggcctggtgcaggccggcggcagcctgaagctgagctgcgtggccagcggcttcaccttcagcaactacgccatggcctggtacaggcaggcccccggcaagcagagggagctgatcgccagctgcagcgccagctgcatctggaccaactacggcgccagcgtgaagggcaggttcaccatgagcctggacaacgccaagaagaccgtgtacctgcagatggacagcctgaagcccgaggacaccgccgtgtactactgcaggaacctggacgccaactactggggccagggcacccaggtgaccgtgagcagc

SEQ ID NO: 31 - protein, 114 aa, Artificial sequence
  Synthetic
QLQLVESGGGLVQAGGSLRLSCAVSISTFTTNGWDWYRQAPGKQRELVALISNDGTTTYTDSVKGRFTISRDGAKNTVYLQMNNLKPEDTAVYYCNTIPPAGSWGQGTQVTVSS

SEQ ID NO: 32 - DNA, 342 bp, Artificial sequence
  Synthetic
cagctgcagctggtggagagcggcggcggcctggtgcaggccggcggcagcctgaggctgagctgcgccgtgagcatcagcaccttcaccaccaacggctgggactggtacaggcaggcccccggcaagcagagggagctggtggccctgatcagcaacgacggcaccaccacctacaccgacagcgtgaagggcaggttcaccatcagcagggacggcgccaagaacaccgtgtacctgcagatgaacaacctgaagcccgaggacaccgccgtgtactactgcaacaccatcccccccgccggcagctggggccagggcacccaggtgaccgtgagcagc

SEQ ID NO: 33 - protein, 114 aa, Artificial sequence
  Synthetic
EVQLVESGGGLVQAGGSLRLSCAASGFRIINDRMAWYRQAPGKQREAVASIDYAGSTTYAEFVKGRFTISRDNTKNMVTLQMNNLEPEDTAVYYCNTAPIARFRGQGTQVTVSS

SEQ ID NO: 34 - DNA, 342 bp, Artificial sequence
  Synthetic
gaggtgcagctggtggagagcggcggcggcctggtgcaggccggcggcagcctgaggctgagctgcgccgccagcggcttcaggatcatcaacgacaggatggcctggtacaggcaggcccccggcaagcagagggaggccgtggccagcatcgactacgccggcagcaccacctacgccgagttcgtgaagggcaggttcaccatcagcagggacaacaccaagaacatggtgaccctgcagatgaacaacctggagcccgaggacaccgccgtgtactactgcaacaccgcccccatcgccaggttcaggggccagggcacccaggtgaccgtgagcagc

SEQ ID NO: 35 - protein, 113 aa, Artificial sequence
  Synthetic
EVQVVESGGGLVQAGGSLRLSCAASTITISSAGVSWYRQAPGKQRELVAIITRGGSTNYADSVKGRFTISRDNAKNTHYLQMYNLKPEDTGIYYCNVVPPTYWGQGTQVTVSS

SEQ ID NO: 36 - DNA, 339 bp, Artificial sequence
  Synthetic
gaggtgcaggtggtggagagcggcggcggcctggtgcaggccggcggcagcctgaggctgagctgcgccgccagcaccatcaccatcagcagcgccggcgtgagctggtacaggcaggcccccggcaagcagagggagctggtggccatcatcaccaggggcggcagcaccaactacgccgacagcgtgaagggcaggttcaccatcagcagggacaacgccaagaacacccactacctgcagatgtacaacctgaagcccgaggacaccggcatctactactgcaacgtggtgccccccacctactggggccagggcacccaggtgaccgtgagcagc

SEQ ID NO: 37 - protein, 114 aa, Artificial sequence
  Synthetic
QVQLVESGGGLVQAGGSLRLSCAASEGTLSSEAMGWHRLAPGKQRESVGFISSGGSTNYRDSVKGRFTISRDNAKNTVYLQMNSLKPEDTGVYFCRIPNSVGPWGQGTQVTVSS

SEQ ID NO: 38 - DNA, 342 bp, Artificial sequence
  Synthetic
caggtgcagctggtggagagcggcggcggcctggtgcaggccggcggcagcctgaggctgagctgcgccgccagcgagggcaccctgagcagcgaggccatgggctggcacaggctggcccccggcaagcagagggagagcgtgggcttcatcagcagcggcggcagcaccaactacagggacagcgtgaagggcaggttcaccatcagcagggacaacgccaagaacaccgtgtacctgcagatgaacagcctgaagcccgaggacaccggcgtgtacttctgcaggatccccaacagcgtgggcccctggggccagggcacccaggtgaccgtgagcagc

SEQ ID NO: 39 - protein, 114 aa, Artificial sequence
  Synthetic
EVQVVESGGGLVQAGGSLRLSCVASGFDFSDSVMGWYRQAPGKQREAVAIISSVGQSNYRDSVQGRFTVSRSNTENTMYLQMDSLKPEDTAIYYCKKFGPGEYWGQGTQVTVSS

SEQ ID NO: 40 - DNA, 342 bp, Artificial sequence
  Synthetic
gaggtgcaggtggtggagagcggcggcggcctggtgcaggccggcggcagcctgaggctgagctgcgtggccagcggcttcgacttcagcgacagcgtgatgggctggtacaggcaggcccccggcaagcagagggaggccgtggccatcatcagcagcgtgggccagagcaactacagggacagcgtgcagggcaggttcaccgtgagcaggagcaacaccgagaacaccatgtacctgcagatggacagcctgaagcccgaggacaccgccatctactactgcaagaagttcggccccggcgagtactggggccagggcacccaggtgaccgtgagcagc

SEQ ID NO: 41 - protein, 115 aa, Artificial sequence
  Synthetic
EVQLVESGGGLVQAGGSLRLSCVVSGSTFSSYAMGWYRQAPGKQRELVAAISSGVSANVADSLKGRFAISRDNAKNAVYLQMNSLKPEDTAVYYCNTLPRSMPYWGKGTLVTVSS

SEQ ID NO: 42 - DNA, 345 bp, Artificial sequence
  Synthetic
gaggtgcagctggtggagagcggcggcggcctggtgcaggccggcggcagcctgaggctgagctgcgtggtgagcggcagcaccttcagcagctacgccatgggctggtacaggcaggcccccggcaagcagagggagctggtggccgccatcagcagcggcgtgagcgccaacgtggccgacagcctgaagggcaggttcgccatcagcagggacaacgccaagaacgccgtgtacctgcagatgaacagcctgaagcccgaggacaccgccgtgtactactgcaacaccctgcccaggagcatgccctactggggcaagggcaccctggtgaccgtgagcagc

SEQ ID NO: 43 - protein, 118 aa, Artificial sequence
  Synthetic
EVQLVESGGGLVQAGGSLRLSCAASGSTLSSYAMGWYRQAPGKQRELVAAISSGGGSTNYRDSVKGRFTISRDNAKNTVYLQMNSLKPEDTAVYYCKSTDYGSLFDSWGQGTQVTVSS

SEQ ID NO: 44 - DNA, 354 bp, Artificial sequence
  Synthetic
gaggtgcagctggtggagagcggcggcggcctggtgcaggccggcggcagcctgaggctgagctgcgccgccagcggcagcaccctgagcagctacgccatgggctggtacaggcaggcccccggcaagcagagggagctggtggccgccatcagcagcggcggcggcagcaccaactacagggacagcgtgaagggcaggttcaccatcagcagggacaacgccaagaacaccgtgtacctgcagatgaacagcctgaagcccgaggacaccgccgtgtactactgcaagagcaccgactacggcagcctgttcgacagctggggccagggcacccaggtgaccgtgagcagc

SEQ ID NO: 45 - protein, 127 aa, Artificial sequence
  Synthetic
EVQVVESGGGTVQAGGSLRLSCAASARTFANAHMAWFRQAPGKEREFLAAITWSGGITDYANSVKGRFTISRDNAENAMYLQMNSLKPEDTAIYICAVDTTSAYDQLWSRQSEYEYWGQGTQVTVSS

SEQ ID NO: 46 - DNA, 381 bp, Artificial sequence
  Synthetic
gaggtgcaggtggtggagagcggcggcggcaccgtgcaggccggcggcagcctgaggctgagctgcgccgccagcgccaggaccttcgccaacgcccacatggcctggttcaggcaggcccccggcaaggagagggagttcctggccgccatcacctggagcggcggcatcaccgactacgccaacagcgtgaagggcaggttcaccatcagcagggacaacgccgagaacgccatgtacctgcagatgaacagcctgaagcccgaggacaccgccatctacatctgcgccgtggacaccaccagcgcctacgaccagctgtggagcaggcagagcgagtacgagtactggggccagggcacccaggtgaccgtgagcagc

SEQ ID NO: 47 - protein, 114 aa, Artificial sequence
  Synthetic
QVQLVESGGGLVQPGGSLRLSCVVSESISVINAMTWYRQAPGKQRELVALISRGGSTNYADSVKGRFTISRDNAKNSVYLQMNSLKPEDTALYYCNVVPPLGSWGQGTQVTVSS

SEQ ID NO: 48 - DNA, 342 bp, Artificial sequence
  Synthetic
caggtgcagctggtggagagcggcggcggcctggtgcagcccggcggcagcctgaggctgagctgcgtggtgagcgagagcatcagcgtgatcaacgccatgacctggtacaggcaggcccccggcaagcagagggagctggtggccctgatcagcaggggcggcagcaccaactacgccgacagcgtgaagggcaggttcaccatcagcagggacaacgccaagaacagcgtgtacctgcagatgaacagcctgaagcccgaggacaccgccctgtactactgcaacgtggtgccccccctgggcagctggggccagggcacccaggtgaccgtgagcagc

SEQ ID NO: 49 - protein, 115 aa, Artificial sequence
  Synthetic
QLQLVESGGGLVRPGGSLRLSCAASGSIFSRSGATWYRQAPGKQRELVALLTRDGHTDYPVVSVKGRFTISKDNAKNTVYLQMNSLQPEDTAVYYCNAIPPLGSWGRGTQVTVSS

SEQ ID NO: 50 - DNA, 345 bp, Artificial sequence
  Synthetic
cagctgcagctggtggagagcggcggcggcctggtgaggcccggcggcagcctgaggctgagctgcgccgccagcggcagcatcttcagcaggagcggcgccacctggtacaggcaggcccccggcaagcagagggagctggtggccctgctgaccagggacggccacaccgactaccccgtggtgagcgtgaagggcaggttcaccatcagcaaggacaacgccaagaacaccgtgtacctgcagatgaacagcctgcagcccgaggacaccgccgtgtactactgcaacgccatcccccccctgggcagctggggcaggggcacccaggtgaccgtgagcagc

SEQ ID NO: 51 - protein, 131 aa, Artificial sequence
  Synthetic
QVQLVESGGGLVQPGGSLRLSCAASGFSLDAYAIGWFRQAPGMGREGVSCISSAGNTDYADSVKGRFAISRDNAKNTVYLQMNSLKPEDSGVYYCARTSEGVYRGRLACALYESAADFRSGGQGTRVTVSS

SEQ ID NO: 52 - DNA, 393 bp, Artificial sequence
  Synthetic
caggtgcagctggtggagagcggcggcggcctggtgcagcccggcggcagcctgaggctgagctgcgccgccagcggcttcagcctggacgcctacgccatcggctggttcaggcaggcccccggcatgggcagggagggcgtgagctgcatcagcagcgccggcaacaccgactacgccgacagcgtgaagggcaggttcgccatcagcagggacaacgccaagaacaccgtgtacctgcagatgaacagcctgaagcccgaggacagcggcgtgtactactgcgccaggaccagcgagggcgtgtacaggggcaggctggcctgcgccctgtacgagagcgccgccgacttcaggagcggcggccagggcaccagggtgaccgtgagcagc

SEQ ID NO: 53 - protein, 113 aa, Artificial sequence
  Synthetic
EVQVVESGGGLVQPGGSLRLSCAASGSGFTINAMTWYRRAPGKERELVAIITNGGITNYADSVKGRFTISRDNAKSTVYLQMDGLEPEDTAVYYCNIVPPVYWGQGTQVTVSS

SEQ ID NO: 54 - DNA, 339 bp, Artificial sequence
  Synthetic
gaggtgcaggtggtggagagcggcggcggcctggtgcagcccggcggcagcctgaggctgagctgcgccgccagcggcagcggcttcaccatcaacgccatgacctggtacaggagggcccccggcaaggagagggagctggtggccatcatcaccaacggcggcatcaccaactacgccgacagcgtgaagggcaggttcaccatcagcagggacaacgccaagagcaccgtgtacctgcagatggacggcctggagcccgaggacaccgccgtgtactactgcaacatcgtgccccccgtgtactggggccagggcacccaggtgaccgtgagcagc

SEQ ID NO: 55 - protein, 125 aa, Artificial sequence
  Synthetic
QVQLVESGGGLVQAGGSLRLSCVASGRFPSIYRMAWFRQAPGKERDFVAAINWGGTATYYEDSVKGRFTISRDNTKNTVWLQMNSLKPEDTAVYYCAAGTGTTYTPQRGDAYGYWGQGTQVTVSS

SEQ ID NO: 56 - DNA, 375 bp, Artificial sequence
  Synthetic
caggtgcagctggtggagagcggcggcggcctggtgcaggccggcggcagcctgaggctgagctgcgtggccagcggcaggttccccagcatctacaggatggcctggttcaggcaggcccccggcaaggagagggacttcgtggccgccatcaactggggcggcaccgccacctactacgaggacagcgtgaagggcaggttcaccatcagcagggacaacaccaagaacaccgtgtggctgcagatgaacagcctgaagcccgaggacaccgccgtgtactactgcgccgccggcaccggcaccacctacaccccccagaggggcgacgcctacggctactggggccagggcacccaggtgaccgtgagcagc

SEQ ID NO: 57 - protein, 114 aa, Artificial sequence
  Synthetic
QLQLVESGGGLVQPGGSLRLSCAASRSISSINTMTWYRYQGPGKERELVALITLGGTTNYADSVKGRFTISRDDAKNTLYLEMNSLKPEDTAVYYCNAVPPFRWGQGTQVTVSS

SEQ ID NO: 58 - DNA, 342 bp, Artificial sequence
  Synthetic
cagctgcagctggtggagagcggcggcggcctggtgcagcccggcggcagcctgaggctgagctgcgccgccagcaggagcatcagcagcatcaacaccatgacctggtacaggtaccagggccccggcaaggagagggagctggtggccctgatcaccctgggcggcaccaccaactacgccgacagcgtgaagggcaggttcaccatcagcagggacgacgccaagaacaccctgtacctggagatgaacagcctgaagcccgaggacaccgccgtgtactactgcaacgccgtgccccccttcaggtggggccagggcacccaggtgaccgtgagcagc

SEQ ID NO: 59 - protein, 115 aa, Artificial sequence
  Synthetic
EVQVVESGGGLVQPGGSLRLSCVASGIIFSSYAMGWYRQAPGKQRELVARISSGGGLYYEDPVKGRFTISRDNARNTVYLQMSSVKPEDTAVYYCNVVPYTPGYWGQGTQVTVSS

SEQ ID NO: 60 - DNA, 345 bp, Artificial sequence
  Synthetic
gaggtgcaggtggtggagagcggcggcggcctggtgcagcccggcggcagcctgaggctgagctgcgtggccagcggcatcatcttcagcagctacgccatgggctggtacaggcaggcccccggcaagcagagggagctggtggccaggatcagcagcggcggcggcctgtactacgaggaccccgtgaagggcaggttcaccatcagcagggacaacgccaggaacaccgtgtacctgcagatgagcagcgtgaagcccgaggacaccgccgtgtactactgcaacgtggtgccctacacccccggctactggggccagggcacccaggtgaccgtgagcagc

SEQ ID NO: 61 - protein, 122 aa, Artificial sequence
  Synthetic
QVQLVESGGFVQAGGSLRLSCAASGRTFSKYAMGWFRQAPGKEREFVAAIRWIGGSTYYADSVKGRFTISRDNDKNTLYLQMNSLKPEDTAVYYCAAGYQAYPEPPWEYDYWGQGTQVTVSS

SEQ ID NO: 62 - DNA, 366 bp, Artificial sequence
  Synthetic
caggtgcagctggtggagagcggcggcttcgtgcaggccggcggcagcctgaggctgagctgcgccgccagcggcaggaccttcagcaagtacgccatgggctggttcaggcaggcccccggcaaggagagggagttcgtggccgccatcaggtggatcggcggcagcacctactacgccgacagcgtgaagggcaggttcaccatcagcagggacaacgacaagaacaccctgtacctgcagatgaacagcctgaagcccgaggacaccgccgtgtactactgcgccgccggctaccaggcctaccccgagcccccctgggagtacgactactggggccagggcacccaggtgaccgtgagcagc

SEQ ID NO: 63 - protein, 8 aa, Artificial sequence
  CDR
ISTFGRTD

SEQ ID NO: 64 - protein, 7 aa, Artificial sequence
  CDR
ISSGGST

SEQ ID NO: 65 - protein, 8 aa, Artificial sequence
  CDR
NTVPPRGS

SEQ ID NO: 66 - protein, 5 aa, Artificial sequence
  CDR
RTDMT

SEQ ID NO: 67 - protein, 16 aa, Artificial sequence
  CDR
RISSGGSTIYADSAKG

SEQ ID NO: 68 - protein, 6 aa, Artificial sequence
  CDR
VPPRGS

SEQ ID NO: 69 - protein, 7 aa, Artificial sequence
  CDR
ISTFGRT

SEQ ID NO: 70 - protein, 5 aa, Artificial sequence
  CDR
SSGGS

SEQ ID NO: 71 - protein, 6 aa, Artificial sequence
  CDR
VPPRGS

SEQ ID NO: 72 - protein, 8 aa, Artificial sequence
  CDR
DDISSIYT

SEQ ID NO: 73 - protein, 7 aa, Artificial sequence
  CDR
VTPGGGT

SEQ ID NO: 74 - protein, 11 aa, Artificial sequence
  CDR
NARHRVTGFAY

SEQ ID NO: 75 - protein, 5 aa, Artificial sequence
  CDR
IYTMA

SEQ ID NO: 76 - protein, 16 aa, Artificial sequence
  CDR
LVTPGGGTNYADSVKG

SEQ ID NO: 77 - protein, 9 aa, Artificial sequence
  CDR
RHRVTGFAY

SEQ ID NO: 78 - protein, 7 aa, Artificial sequence
  CDR
DDISSIY

SEQ ID NO: 79 - protein, 5 aa, Artificial sequence
  CDR
TPGGG

SEQ ID NO: 80 - protein, 9 aa, Artificial sequence
  CDR
RHRVTGFAY

SEQ ID NO: 81 - protein, 8 aa, Artificial sequence
  CDR
GSTFSSYA

SEQ ID NO: 82 - protein, 7 aa, Artificial sequence
  CDR
ISSGGST

SEQ ID NO: 83 - protein, 14 aa, Artificial sequence
  CDR
NTDWGNGFSAEYDY

SEQ ID NO: 84 - protein, 5 aa, Artificial sequence
  CDR
SYAMT

SEQ ID NO: 85 - protein, 16 aa, Artificial sequence
  CDR
AISSGGSTNYAASVKG

SEQ ID NO: 86 - protein, 12 aa, Artificial sequence
  CDR
DWGNGFSAEYDY

SEQ ID NO: 87 - protein, 7 aa, Artificial sequence
  CDR
GSTFSSY

SEQ ID NO: 88 - protein, 5 aa, Artificial sequence
  CDR
SSGGS

SEQ ID NO: 89 - protein, 12 aa, Artificial sequence
  CDR
DWGNGFSAEYDY

SEQ ID NO: 90 - protein, 8 aa, Artificial sequence
  CDR
QSISSINA

SEQ ID NO: 91 - protein, 7 aa, Artificial sequence
  CDR
IFNNGRT

SEQ ID NO: 92 - protein, 8 aa, Artificial sequence
  CDR
NIVPPLRN

SEQ ID NO: 93 - protein, 5 aa, Artificial sequence
  CDR
INAMD

SEQ ID NO: 94 - protein, 16 aa, Artificial sequence
  CDR
IIFNNGRTNYADSVKG

SEQ ID NO: 95 - protein, 6 aa, Artificial sequence
  CDR
VPPLRN

SEQ ID NO: 96 - protein, 7 aa, Artificial sequence
  CDR
QSISSIN

SEQ ID NO: 97 - protein, 5 aa, Artificial sequence
  CDR
FNNGR

SEQ ID NO: 98 - protein, 6 aa, Artificial sequence
  CDR
VPPLRN

SEQ ID NO: 99 - protein, 8 aa, Artificial sequence
  CDR
GTISSYDV

SEQ ID NO: 100 - protein, 7 aa, Artificial sequence
  CDR
IGTDRWL

SEQ ID NO: 101 - protein, 10 aa, Artificial sequence
  CDR
YTFQHTVGPR

SEQ ID NO: 102 - protein, 5 aa, Artificial sequence
  CDR
YDVVG

SEQ ID NO: 103 - protein, 16 aa, Artificial sequence
  CDR
LIGTDRWLNLGDFAKG

SEQ ID NO: 104 - protein, 8 aa, Artificial sequence
  CDR
FQHTVGPR

SEQ ID NO: 105 - protein, 7 aa, Artificial sequence
  CDR
GTISSYD

SEQ ID NO: 106 - protein, 5 aa, Artificial sequence
  CDR
GTDRW

SEQ ID NO: 107 - protein, 8 aa, Artificial sequence
  CDR
FQHTVGPR

SEQ ID NO: 108 - protein, 8 aa, Artificial sequence
  CDR
GFTFSNYA

SEQ ID NO: 109 - protein, 8 aa, Artificial sequence
  CDR
CSASCIWT

SEQ ID NO: 110 - protein, 7 aa, Artificial sequence
  CDR
RNLDANY

SEQ ID NO: 111 - protein, 5 aa, Artificial sequence
  CDR
NYAMA

SEQ ID NO: 112 - protein, 17 aa, Artificial sequence
  CDR
SCSASCIWTNYGASVKG

SEQ ID NO: 113 - protein, 5 aa, Artificial sequence
  CDR
LDANY

SEQ ID NO: 114 - protein, 7 aa, Artificial sequence
  CDR
GFTFSNY

SEQ ID NO: 115 - protein, 6 aa, Artificial sequence
  CDR
SASCIW

SEQ ID NO: 116 - protein, 5 aa, Artificial sequence
  CDR
LDANY

SEQ ID NO: 117 - protein, 8 aa, Artificial sequence
  CDR
ISTFTTNG

SEQ ID NO: 118 - protein, 7 aa, Artificial sequence
  CDR
ISNDGTT

SEQ ID NO: 119 - protein, 8 aa, Artificial sequence
  CDR
NTIPPAGS

SEQ ID NO: 120 - protein, 5 aa, Artificial sequence
  CDR
TNGWD

SEQ ID NO: 121 - protein, 16 aa, Artificial sequence
  CDR
LISNDGTTTYTDSVKG

SEQ ID NO: 122 - protein, 6 aa, Artificial sequence
  CDR
IPPAGS

SEQ ID NO: 123 - protein, 7 aa, Artificial sequence
  CDR
ISTFTTN

SEQ ID NO: 124 - protein, 5 aa, Artificial sequence
  CDR
SNDGT

SEQ ID NO: 125 - protein, 6 aa, Artificial sequence
  CDR
IPPAGS

SEQ ID NO: 126 - protein, 8 aa, Artificial sequence
  CDR
GFRIINDR

SEQ ID NO: 127 - protein, 7 aa, Artificial sequence
  CDR
IDYAGST

SEQ ID NO: 128 - protein, 8 aa, Artificial sequence
  CDR
NTAPIARF

SEQ ID NO: 129 - protein, 5 aa, Artificial sequence
  CDR
NDRMA

SEQ ID NO: 130 - protein, 16 aa, Artificial sequence
  CDR
SIDYAGSTTYAEFVKG

SEQ ID NO: 131 - protein, 6 aa, Artificial sequence
  CDR
APIARF

SEQ ID NO: 132 - protein, 7 aa, Artificial sequence
  CDR
GFRIIND

SEQ ID NO: 133 - protein, 5 aa, Artificial sequence
  CDR
DYAGS

SEQ ID NO: 134 - protein, 6 aa, Artificial sequence
  CDR
APIARF

SEQ ID NO: 135 - protein, 8 aa, Artificial sequence
  CDR
TITISSAG

SEQ ID NO: 136 - protein, 7 aa, Artificial sequence
  CDR
ITRGGST

SEQ ID NO: 137 - protein, 7 aa, Artificial sequence
  CDR
NVVPPTY

SEQ ID NO: 138 - protein, 5 aa, Artificial sequence
  CDR
SAGVS

SEQ ID NO: 139 - protein, 16 aa, Artificial sequence
  CDR
IITRGGSTNYADSVKG

SEQ ID NO: 140 - protein, 5 aa, Artificial sequence
  CDR
VPPTY

SEQ ID NO: 141 - protein, 7 aa, Artificial sequence
  CDR
TITISSA

SEQ ID NO: 142 - protein, 5 aa, Artificial sequence
  CDR
TRGGS

SEQ ID NO: 143 - protein, 5 aa, Artificial sequence
  CDR
VPPTY

SEQ ID NO: 144 - protein, 8 aa, Artificial sequence
  CDR
EGTLSSEA

SEQ ID NO: 145 - protein, 7 aa, Artificial sequence
  CDR
ISSGGST

SEQ ID NO: 146 - protein, 8 aa, Artificial sequence
  CDR
RIPNSVGP

SEQ ID NO: 147 - protein, 5 aa, Artificial sequence
  CDR
SEAMG

SEQ ID NO: 148 - protein, 16 aa, Artificial sequence
  CDR
FISSGGSTNYRDSVKG

SEQ ID NO: 149 - protein, 6 aa, Artificial sequence
  CDR
PNSVGP

SEQ ID NO: 150 - protein, 7 aa, Artificial sequence
  CDR
EGTLSSE

SEQ ID NO: 151 - protein, 5 aa, Artificial sequence
  CDR
SSGGS

SEQ ID NO: 152 - protein, 6 aa, Artificial sequence
  CDR
PNSVGP

SEQ ID NO: 153 - protein, 8 aa, Artificial sequence
  CDR
GFDFSDSV

SEQ ID NO: 154 - protein, 7 aa, Artificial sequence
  CDR
ISSVGQS

SEQ ID NO: 155 - protein, 8 aa, Artificial sequence
  CDR
KKFGPGEY

SEQ ID NO: 156 - protein, 5 aa, Artificial sequence
  CDR
DSVMG

SEQ ID NO: 157 - protein, 16 aa, Artificial sequence
  CDR
IISSVGQSNYRDSVQG

SEQ ID NO: 158 - protein, 6 aa, Artificial sequence
  CDR
FGPGEY

SEQ ID NO: 159 - protein, 7 aa, Artificial sequence
  CDR
GFDFSDS

SEQ ID NO: 160 - protein, 5 aa, Artificial sequence
  CDR
SSVGQ

SEQ ID NO: 161 - protein, 6 aa, Artificial sequence
  CDR
FGPGEY

SEQ ID NO: 162 - protein, 8 aa, Artificial sequence
  CDR
GSTFSSYA

SEQ ID NO: 163 - protein, 7 aa, Artificial sequence
  CDR
ISSGVSA

SEQ ID NO: 164 - protein, 9 aa, Artificial sequence
  CDR
NTLPRSMPY

SEQ ID NO: 165 - protein, 5 aa, Artificial sequence
  CDR
SYAMG

SEQ ID NO: 166 - protein, 16 aa, Artificial sequence
  CDR
AISSGVSANVADSLKG

SEQ ID NO: 167 - protein, 7 aa, Artificial sequence
  CDR
LPRSMPY

SEQ ID NO: 168 - protein, 7 aa, Artificial sequence
  CDR
GSTFSSY

SEQ ID NO: 169 - protein, 5 aa, Artificial sequence
  CDR
SSGVS

SEQ ID NO: 170 - protein, 7 aa, Artificial sequence
  CDR
LPRSMPY

SEQ ID NO: 171 - protein, 8 aa, Artificial sequence
  CDR
GSTLSSYA

SEQ ID NO: 172 - protein, 8 aa, Artificial sequence
  CDR
ISSGGGST

SEQ ID NO: 173 - protein, 11 aa, Artificial sequence
  CDR
KSTDYGSLFDS

SEQ ID NO: 174 - protein, 5 aa, Artificial sequence
  CDR
SYAMG

SEQ ID NO: 175 - protein, 17 aa, Artificial sequence
  CDR
AISSGGGSTNYRDSVKG

SEQ ID NO: 176 - protein, 9 aa, Artificial sequence
  CDR
TDYGSLFDS

SEQ ID NO: 177 - protein, 7 aa, Artificial sequence
  CDR
GSTLSSY

SEQ ID NO: 178 - protein, 6 aa, Artificial sequence
  CDR
SSGGGS

SEQ ID NO: 179 - protein, 9 aa, Artificial sequence
  CDR
TDYGSLFDS

SEQ ID NO: 180 - protein, 8 aa, Artificial sequence
  CDR
ARTFANAH

SEQ ID NO: 181 - protein, 8 aa, Artificial sequence
  CDR
ITWSGGIT

SEQ ID NO: 182 - protein, 20 aa, Artificial sequence
  CDR
AVDTTSAYDQLWSRQSEYEY

SEQ ID NO: 183 - protein, 5 aa, Artificial sequence
  CDR
NAHMA

SEQ ID NO: 184 - protein, 17 aa, Artificial sequence
  CDR
AITWSGGITDYANSVKG

SEQ ID NO: 185 - protein, 18 aa, Artificial sequence
  CDR
DTTSAYDQLWSRQSEYEY

SEQ ID NO: 186 - protein, 7 aa, Artificial sequence
  CDR
ARTFANA

SEQ ID NO: 187 - protein, 6 aa, Artificial sequence
  CDR
TWSGGI

SEQ ID NO: 188 - protein, 18 aa, Artificial sequence
  CDR
DTTSAYDQLWSRQSEYEY

SEQ ID NO: 189 - protein, 8 aa, Artificial sequence
  CDR
ESISVINA

SEQ ID NO: 190 - protein, 7 aa, Artificial sequence
  CDR
ISRGGST

SEQ ID NO: 191 - protein, 8 aa, Artificial sequence
  CDR
NVVPPLGS

SEQ ID NO: 192 - protein, 5 aa, Artificial sequence
  CDR
INAMT

SEQ ID NO: 193 - protein, 16 aa, Artificial sequence
  CDR
LISRGGSTNYADSVKG

SEQ ID NO: 194 - protein, 6 aa, Artificial sequence
  CDR
VPPLGS

SEQ ID NO: 195 - protein, 7 aa, Artificial sequence
  CDR
ESISVIN

SEQ ID NO: 196 - protein, 5 aa, Artificial sequence
  CDR
SRGGS

SEQ ID NO: 197 - protein, 6 aa, Artificial sequence
  CDR
VPPLGS

SEQ ID NO: 198 - protein, 8 aa, Artificial sequence
  CDR
GSIFSRSG

SEQ ID NO: 199 - protein, 7 aa, Artificial sequence
  CDR
LTRDGHT

SEQ ID NO: 200 - protein, 8 aa, Artificial sequence
  CDR
NAIPPLGS

SEQ ID NO: 201 - protein, 5 aa, Artificial sequence
  CDR
RSGAT

SEQ ID NO: 202 - protein, 17 aa, Artificial sequence
  CDR
LLTRDGHTDYPVVSVKG

SEQ ID NO: 203 - protein, 6 aa, Artificial sequence
  CDR
IPPLGS

SEQ ID NO: 204 - protein, 7 aa, Artificial sequence
  CDR
GSIFSRS

SEQ ID NO: 205 - protein, 5 aa, Artificial sequence
  CDR
TRDGH

SEQ ID NO: 206 - protein, 6 aa, Artificial sequence
  CDR
IPPLGS

SEQ ID NO: 207 - protein, 8 aa, Artificial sequence
  CDR
GFSLDAYA

SEQ ID NO: 208 - protein, 7 aa, Artificial sequence
  CDR
ISSAGNT

SEQ ID NO: 209 - protein, 12 aa, Artificial sequence
  CDR
ARTSEGVYRGRL

SEQ ID NO: 210 - protein, 5 aa, Artificial sequence
  CDR
AYAIG

SEQ ID NO: 211 - protein, 16 aa, Artificial sequence
  CDR
CISSAGNTDYADSVKG

SEQ ID NO: 212 - protein, 23 aa, Artificial sequence
  CDR
TSEGVYRGRLACALYESAADFRS

SEQ ID NO: 213 - protein, 7 aa, Artificial sequence
  CDR
GFSLDAY

SEQ ID NO: 214 - protein, 5 aa, Artificial sequence
  CDR
SSAGN

SEQ ID NO: 215 - protein, 23 aa, Artificial sequence
  CDR
TSEGVYRGRLACALYESAADFRS

SEQ ID NO: 216 - protein, 8 aa, Artificial sequence
  CDR
GSGFTINA

SEQ ID NO: 217 - protein, 7 aa, Artificial sequence
  CDR
ITNGGIT

SEQ ID NO: 218 - protein, 7 aa, Artificial sequence
  CDR
NIVPPVY

SEQ ID NO: 219 - protein, 5 aa, Artificial sequence
  CDR
INAMT

SEQ ID NO: 220 - protein, 16 aa, Artificial sequence
  CDR
IITNGGITNYADSVKG

SEQ ID NO: 221 - protein, 5 aa, Artificial sequence
  CDR
VPPVY

SEQ ID NO: 222 - protein, 7 aa, Artificial sequence
  CDR
GSGFTIN

SEQ ID NO: 223 - protein, 5 aa, Artificial sequence
  CDR
TNGGI

SEQ ID NO: 224 - protein, 5 aa, Artificial sequence
  CDR
VPPVY

SEQ ID NO: 225 - protein, 8 aa, Artificial sequence
  CDR
GRFPSIYR

SEQ ID NO: 226 - protein, 8 aa, Artificial sequence
  CDR
INWGGTAT

SEQ ID NO: 227 - protein, 18 aa, Artificial sequence
  CDR
AAGTGTTYTPQRGDAYGY

SEQ ID NO: 228 - protein, 5 aa, Artificial sequence
  CDR
IYRMA

SEQ ID NO: 229 - protein, 17 aa, Artificial sequence
  CDR
AINWGGTATYYEDSVKG

SEQ ID NO: 230 - protein, 16 aa, Artificial sequence
  CDR
GTGTTYTPQRGDAYGY

SEQ ID NO: 231 - protein, 7 aa, Artificial sequence
  CDR
GRFPSIY

SEQ ID NO: 232 - protein, 6 aa, Artificial sequence
  CDR
NWGGTA

SEQ ID NO: 233 - protein, 16 aa, Artificial sequence
  CDR
GTGTTYTPQRGDAYGY

SEQ ID NO: 234 - protein, 8 aa, Artificial sequence
  CDR
RSISSINT

SEQ ID NO: 235 - protein, 7 aa, Artificial sequence
  CDR
LITLGGT

SEQ ID NO: 236 - protein, 7 aa, Artificial sequence
  CDR
NAVPPFR

SEQ ID NO: 237 - protein, 5 aa, Artificial sequence
  CDR
INTMT

SEQ ID NO: 238 - protein, 16 aa, Artificial sequence
  CDR
LITLGGTTNYADSVKG

SEQ ID NO: 239 - protein, 5 aa, Artificial sequence
  CDR
VPPFR

SEQ ID NO: 240 - protein, 7 aa, Artificial sequence
  CDR
RSISSIN

SEQ ID NO: 241 - protein, 5 aa, Artificial sequence
  CDR
TLGGT

SEQ ID NO: 242 - protein, 5 aa, Artificial sequence
  CDR
VPPFR

SEQ ID NO: 243 - protein, 8 aa, Artificial sequence
  CDR
GIIFSSYA

SEQ ID NO: 244 - protein, 7 aa, Artificial sequence
  CDR
ISSGGGL

SEQ ID NO: 245 - protein, 9 aa, Artificial sequence
  CDR
NVVPYTPGY

SEQ ID NO: 246 - protein, 5 aa, Artificial sequence
  CDR
SYAMG

SEQ ID NO: 247 - protein, 16 aa, Artificial sequence
  CDR
RISSGGGLYYEDPVKG

SEQ ID NO: 248 - protein, 7 aa, Artificial sequence
  CDR
VPYTPGY

SEQ ID NO: 249 - protein, 7 aa, Artificial sequence
  CDR
GIIFSSY

SEQ ID NO: 250 - protein, 5 aa, Artificial sequence
  CDR
SSGGG

SEQ ID NO: 251 - protein, 7 aa, Artificial sequence
  CDR
VPYTPGY

SEQ ID NO: 252 - protein, 8 aa, Artificial sequence
  CDR
GRTFSKYA

SEQ ID NO: 253 - protein, 8 aa, Artificial sequence
  CDR
IRWIGGST

SEQ ID NO: 254 - protein, 16 aa, Artificial sequence
  CDR
AAGYQAYPEPPWEYDY

SEQ ID NO: 255 - protein, 5 aa, Artificial sequence
  CDR
KYAMG

SEQ ID NO: 256 - protein, 17 aa, Artificial sequence
  CDR
AIRWIGGSTYYADSVKG

SEQ ID NO: 257 - protein, 14 aa, Artificial sequence
  CDR
GYQAYPEPPWEYDY

SEQ ID NO: 258 - protein, 7 aa, Artificial sequence
  CDR
GRTFSKY

SEQ ID NO: 259 - protein, 6 aa, Artificial sequence
  CDR
RWIGGS

SEQ ID NO: 260 - protein, 14 aa, Artificial sequence
  CDR
GYQAYPEPPWEYDY

(19) United States
(12) Patent Application Publication
(10) Pub. No.: US 2024/0101686 A1
(43) Pub. Date: Mar. 28, 2024

(54) ANTI-EGFR NANOBODY AND USE THEREOF

(71) Applicant: SIMCERE ZAIMING PHARMACEUTICAL CO., LTD., Jiangsu (CN)

(72) Inventors: Peipei Wei, Shanghai (CN); Cuiqing Yang, Shanghai (CN); Zhuoxiao Cao, Shanghai (CN); Renhong Tang, Shanghai (CN); Jinsheng Ren, Nanjing, Jiangsu (CN)

(21) Appl. No.: 18/266,125

(22) PCT Filed: Dec. 8, 2021
(86) PCT No.: PCT/CN2021/136362

§ 371 Date: Jun. 8, 2023

(30) Foreign Application Priority Data
Dec. 9, 2020 (CN) 202011425866.9

(51) Int. Cl. C07K 16/28; A61K 45/06

(52) U.S. Cl. CPC C07K 16/2863; A61K 45/06; C07K 2317/24; C07K 2317/31; C07K 2317/565; C07K 2317/569; C07K 2317/92

(57) ABSTRACT

An EGFR nanobody, and a preparation method therefor and the use thereof. The EGFR nanobody has high affinity for a wild-type EGRF protein, and also recognizes the EGRFvIII protein.

The present disclosure claims the right of priority of Chinese patent application no. 202011425866.9 titled “ANTI-EGFR NANOBODY AND USE THEREOF” and filed with the China Patent Office on Dec. 9, 2020, which is incorporated in the present disclosure by reference in its entirety.

### TECHNICAL FIELD

The present invention relates to the fields of bioengineering and biomedicine, and mainly relates to a nanobody targeting EGFR or an antigen-binding fragment thereof, and an encoding nucleic acid, an expression vector and an expression cell, a preparation method, a pharmaceutical composition therefor, and their use for treating diseases, such as use for treating tumors.

### BACKGROUND ART

Epidermal growth factor receptor (EGFR) is a multifunctional glycoprotein widely distributed on the cell membrane of various tissues in the human body. It is a homologue of the oncogene of avian erythroblastic leukemia virus (v-erb-b) and one of the four members of HER/ErbB family, so it is also called HER1 or ErbB-1. Overexpression of EGFR has been found in a variety of tumors, including bladder cancer, brain cancer, head and neck cancer, pancreatic cancer, lung cancer, breast cancer, ovarian cancer, colon cancer, prostate cancer, and kidney cancer (Atalay et al., Novel therapeutic strategies targeting the epidermal growth factor receptor (EGFR) family and its downstream effectors in breast cancer. Ann. Oncology, 2003, 14: 1346-1363; Herbst and Shin, Monoclone antibodies target epidermal growth factor receptor positive cancer therapy. American Cancer Society, 2000, 1593-1611). Therefore, EGFR is a promising target molecule for the treatment of tumors.

EGFR consists of three parts: (1) Extracellular domain (ECD): extracellular region is at the NH2 end, is a ligand binding region, has a total of 621 amino acid residues, and consists of four subdomains I, II, III, and IV (or correspondingly called L1, S1/CR1, L2, S2/CR2 subdomains) (Bishayee S., Role of conformational alteration in the epidermal growth factor receptor (EGFR) function. BiochemPharmacol, 2000, 60(8): 1217-1223). Domains II and IV have high homology and are dimerization binding regions. Domain II has the characteristics of (3-hairpin or dimerization arm. Normally, the extracellular domain of the receptor is in a closed, inactive conformation in its equilibrium state, and the (3-hairpin in domain II contacts with the conserved residue in domain IV intramolecularly, which prevents dimerization. Upon binding of a ligand to a corresponding receptor, the orientation of domains I and III changes, resulting in the exposure of dimerization arm that allows dimerization with other receptors. (2) Transmembrane (TM) domain: transmembrane domain is a hydrophobic region composed of 23 amino acid residues and is a single-stranded α-helix (Abe Y, Odaka M, Inagaki F, et al., Disulfide bond structure of human epidermal growth factor receptor. J Biol Chem, 1998, 273(18): 11150-11157). (3) Intracellular domain (ICD): ICD has a total of 542 amino acid residues, and can be divided into two parts, i.e., a tyrosine kinase domain and a C-terminal domain. The former has an adenosine triphosphate (ATP) binding site, and the phosphate group can be transferred upon ATP binding; the latter has multiple tyrosine residues, which can be phosphorylated and directly participate in intracellular signal transduction (Nam Y. Lee, Structure and dynamics of the epidermal growth factor receptor C-terminal phosphorylation domain. Protein Sci. 2006, 15(5): 1142-1152).

Ligands of EGFR include EGF, TGFA/TGF-alpha, amphiregulin, epigen/EPGN, BTC/betacellulin, epiregulin/EREG and HBEGF/heparin-binding EGF. The binding of the receptor and the ligand would trigger EGFR to form a homodimer or a heterodimer, which would lead to autophosphorylation of the intracellular domain and further activate complex downstream signal cascade reactions, mainly including the following signaling pathways: RAS-RAF-MEK-ERK signaling pathway, phosphatidylinositol 3-kinase (PI3K) signaling pathway, PLC gamma-PKC signaling pathway and STATs modules signaling pathway. EGFR can regulate a variety of cellular physiological processes through these tyrosine kinase-mediated signal transduction pathways, and the cellular physiological processes mainly include cell proliferation and differentiation, cell survival and apoptosis, angiogenesis, and cell mitosis and cell metastasis (Atalay et al., Novel therapeutic strategies targeting the epidermal growth factor receptor (EGFR) family and its downstream effectors in breast cancer. Ann. Oncology, 2003, 14: 1346-1363; Herbst and Shin, Monoclone antibodies target epidermal growth factor receptor positive cancer therapy. American Cancer Society, 2000, 1593-1611; Modjtahedi et al., Phase I trial and tumor localization of the anti-EGFR monoclonal antibody ICR62 in head and neck or lung cancer Br. J. Cancer, 1996, 73: 228-235).

Studies have shown that overexpression of EGFR can promote the transformation of normal cells and the metastasis of malignant tumors. Overexpression is often associated with gene amplification (Towia A. Libermann et al., Amplification, enhanced expression and possible rearrangement of EGF receptor gene in primary human brain tumors of glial origin, 1985, Nature 313:144-147). Rearrangement of the EGFR gene is evident in many tumors with gene amplification, giving rise to EGFR variants (Maiden et al, Selective Amplification of the Cytoplasmic Domain of the Epidermal Growth Factor Receptor Gene in Glioblastoma Multiforme, 1988, Cancer Research 4: 2711-2714). Currently there are eight main mutant types: (1) EGFRvI lacking most of the extracellular domain of EGFR; (2) EGFRvII consisting of an 83 amino acid in-frame deletion in the extracellular domain of EGFR; (3) EGFRvIII consisting of a 267 amino acid in-frame deletion in the extracellular domain of EGFR; (4) EGFRvIV comprising a deletion in the cytoplasmic domain of EGFR; (5) EGFRvV comprising a deletion in the cytoplasmic domain of EGFR; (6) EGFR.TDM/2-7 comprising a duplication of exon 2-7 in the extracellular domain of EGFR; (7) EGFR.TDM/18-25 comprising a duplication of exon 18-26 in the tyrosine kinase domain of EGFR; (8) EGFR. TDM/18-26 comprising a duplication of exon 18-26 in the tyrosine kinase domain of EGFR (Kuan et al., EGF mutant receptor villas a molecular target in cancer therapy, Endocr relat cancer 8-96, 2001; Wang Chengxing, et al., Tumor therapy targeting EGFR and its mutants, Foreign Medical Sciences, Section of Pathophysiology and Clinical Medicine 2000, 20(2): 137-140). Among them, the EGFRvIII mutant type is the most common variant of the epidermal growth factor (EGF) receptor in human cancers, also known as de2-7 EGFR, ΔFGFR, or Δ7-7 (Olapade-Olaopa E O, Moscatello D K, et al., Evidence for the differential expression of a variant EGF receptor protein in human prostate cancer, Br. J., 2000 Cancer. 82(1): 86-94). 801 nucleotides of exon 2-7 are deleted in the mature EGFRvIII mRNA, and the corresponding EGFR protein has 267 amino acids (6-273) deleted, and has a glycine residue inserted at the same time, forming a unique connecting peptide (A J Wong et al., Structural Alterations of the Epidermal Growth Factor Receptor Gene in Human Glioma. Proc Natl Acad Sci USA 1992, 89(7): 2965-9; Yamazaki et al., mutation within the ligand binding domain is responsible for activation of epidermal growth factor receptor gene in human brain tumors, Jpn. J. Cancer Res., 1990, 81: 773-9; Yamazaki et al., Amplification of the structurally and functionally altered epidermal growth factor receptor gene (c-erbB) in human brain tumor, Mol. Cell. Biol, 1988, 8(4): 1816-20; Sugawa et al. Identical Splicing of Aberrant Epidermal Growth Factor Receptor Transcripts From Amplified Rearranged Genes in Human Glioblastomas, Proc Natl Acad Sci USA 1990 November; 87(21): 8602-6).

Expression of EGFRvIII has been reported in a variety of tumor types, including glioma, breast, lung, ovarian, and prostate cancers (Wikstrand et al., cell surface localization and density of the tumor-associated variant of the epidermal growth factor receptor, EGFRvIII, Cancer Res. 57, 4130-40, 1997; Olapade-Olaopa et al., Evidence for the differential expression of the variant EGF receptor protein in human prostate cancer, Br. J. Cancer. 82, 86-94, 2000; C J Wikstrand et al., Monoclonal Antibodies Against EGFRvIII Are Tumor Specific and React With Breast and Lung Carcinomas and Malignant Gliomas, Cancer Res, 1995, 55(14): 3140-3148; Garcia de Palazzo et al., Expression of mutated epidermal growth factor receptor by non-small cell lung carcinomas. Cancer Res, 1993, 53: 3217-3220). EGFRvIII cannot bind to ligand, but it is in a persistent low activation state. The mechanism of EGFRvIII in glioma is not completely clear, but according to existing reports, EGFRvIII can reduce the apoptosis of glioma cells and slightly increase the proliferation of glioma cells (M Nagane et al. A Common Mutant Epidermal Growth Factor Receptor Confers Enhanced Tumorigenicity on Human Glioblastoma Cells by Increasing Proliferation and Reducing Apoptosis. Cancer Res1996, 56(21): 5079-5086). EGFRvIII is specifically expressed in tumor tissues but not in normal tissues, so it is a highly specific target in antibody therapy (Henriqueta A C Silva et al. Molecular Detection of EGFRvIII-positive Cells in the Peripheral Blood of Breast Cancer Patients, Eur J Cancer. 2006, 42(15): 2617-2622).

Nanobody (Nb) is a genetically engineered antibody containing only a single domain. In 1993, Belgian scientist Hamers-Casterman C discovered a natural heavy chain antibody containing only heavy chains and no light chains in camel blood (Hamers-Casterman C, Atarhouch T, Muyldermans S, Robinson G, Hamers C, Songa E B, et al. Naturally occurring antibodies devoid of light chains. Nature. 363(6428): 446-8(1993).), although the heavy chain antibody lacks light chains compared with an ordinary antibody, they still retain the ability to bind an antigen. After cloning the variable region of the heavy chain antibody in camel, the obtained single domain antibody (sdAb) consisting of only one heavy chain variable region is called nanobody or VHH antibody (variable heavy chain domain of a heavy chain antibody). Not only the nanobody has a molecular weight which is only 1/10 of that of an ordinary antibody, but also has more flexible chemical properties, good stability, high solubility, easy expression, high tumor tissue penetration, and easy coupling to other molecules. Therefore, the application of nanobody technology to develop a therapeutic antibody against EGFR and EGFRvIII has broad prospects.

### SUMMARY OF THE INVENTION

The present invention provides a nanobody or an antigen-binding fragment that specifically binds to EGFR and EGFRvIII, a nucleic acid encoding the antibody and the antigen-binding fragment, a pharmaceutical composition and a kit comprising the antibody and the antigen-binding fragment, and they can be used in the preparation of drugs for treating tumors, etc.

In some embodiments, the nanobody or the antigen-binding fragment that specifically binds EGFR and EGFRvIII, the nanobody or the antigen-binding fragment comprises a combination of CDRs, the combination of CDRs comprises: CDR1, CDR2, and CDR3; the CDR1, CDR2 and CDR3 have any sequence combination selected from the following or a sequence combination with 1, 2, 3 or more amino acid insertions, deletions and/or substitutions compared to the sequence combination:

| | SEQ ID NO. | | |
|---|---|---|---|
| No. | CDR1 | CDR2 | CDR3 |
| VH1 | SEQ ID NO. 63 | SEQ ID NO. 64 | SEQ ID NO. 65 |
| VH2 | SEQ ID NO. 66 | SEQ ID NO. 67 | SEQ ID NO. 68 |
| VH3 | SEQ ID NO. 69 | SEQ ID NO. 70 | SEQ ID NO. 71 |
| VH4 | SEQ ID NO. 72 | SEQ ID NO. 73 | SEQ ID NO. 74 |
| VH5 | SEQ ID NO. 75 | SEQ ID NO. 76 | SEQ ID NO. 77 |
| VH6 | SEQ ID NO. 78 | SEQ ID NO. 79 | SEQ ID NO. 80 |
| VH7 | SEQ ID NO. 81 | SEQ ID NO. 82 | SEQ ID NO. 83 |
| VH8 | SEQ ID NO. 84 | SEQ ID NO. 85 | SEQ ID NO. 86 |
| VH9 | SEQ ID NO. 87 | SEQ ID NO. 88 | SEQ ID NO. 89 |
| VH10 | SEQ ID NO. 90 | SEQ ID NO. 91 | SEQ ID NO. 92 |
| VH11 | SEQ ID NO. 93 | SEQ ID NO. 94 | SEQ ID NO. 95 |
| VH12 | SEQ ID NO. 96 | SEQ ID NO. 97 | SEQ ID NO. 98 |
| VH13 | SEQ ID NO. 99 | SEQ ID NO. 100 | SEQ ID NO. 101 |
| VH14 | SEQ ID NO. 102 | SEQ ID NO. 103 | SEQ ID NO. 104 |
| VH15 | SEQ ID NO. 105 | SEQ ID NO. 106 | SEQ ID NO. 107 |
| VH16 | SEQ ID NO. 108 | SEQ ID NO. 109 | SEQ ID NO. 110 |
| VH17 | SEQ ID NO. 111 | SEQ ID NO. 112 | SEQ ID NO. 113 |
| VH18 | SEQ ID NO. 114 | SEQ ID NO. 115 | SEQ ID NO. 116 |
| VH19 | SEQ ID NO. 117 | SEQ ID NO. 118 | SEQ ID NO. 119 |
| VH20 | SEQ ID NO. 120 | SEQ ID NO. 121 | SEQ ID NO. 122 |
| VH21 | SEQ ID NO. 123 | SEQ ID NO. 124 | SEQ ID NO. 125 |
| VH22 | SEQ ID NO. 126 | SEQ ID NO. 127 | SEQ ID NO. 128 |
| VH23 | SEQ ID NO. 129 | SEQ ID NO. 130 | SEQ ID NO. 131 |
| VH24 | SEQ ID NO. 132 | SEQ ID NO. 133 | SEQ ID NO. 134 |
| VH25 | SEQ ID NO. 135 | SEQ ID NO. 136 | SEQ ID NO. 137 |
| VH26 | SEQ ID NO. 138 | SEQ ID NO. 139 | SEQ ID NO. 140 |
| VH27 | SEQ ID NO. 141 | SEQ ID NO. 142 | SEQ ID NO. 143 |
| VH28 | SEQ ID NO. 144 | SEQ ID NO. 145 | SEQ ID NO. 146 |
| VH29 | SEQ ID NO. 147 | SEQ ID NO. 148 | SEQ ID NO. 149 |
| VH30 | SEQ ID NO. 150 | SEQ ID NO. 151 | SEQ ID NO. 152 |
| VH31 | SEQ ID NO. 153 | SEQ ID NO. 154 | SEQ ID NO. 155 |
| VH32 | SEQ ID NO. 156 | SEQ ID NO. 157 | SEQ ID NO. 158 |
| VH33 | SEQ ID NO. 159 | SEQ ID NO. 160 | SEQ ID NO. 161 |
| VH34 | SEQ ID NO. 162 | SEQ ID NO. 163 | SEQ ID NO. 164 |
| VH35 | SEQ ID NO. 165 | SEQ ID NO. 166 | SEQ ID NO. 167 |
| VH36 | SEQ ID NO. 168 | SEQ ID NO. 169 | SEQ ID NO. 170 |
| VH37 | SEQ ID NO. 171 | SEQ ID NO. 172 | SEQ ID NO. 173 |
| VH38 | SEQ ID NO. 174 | SEQ ID NO. 175 | SEQ ID NO. 176 |
| VH39 | SEQ ID NO. 177 | SEQ ID NO. 178 | SEQ ID NO. 179 |
| VH40 | SEQ ID NO. 180 | SEQ ID NO. 181 | SEQ ID NO. 182 |
| VH41 | SEQ ID NO. 183 | SEQ ID NO. 184 | SEQ ID NO. 185 |
| VH42 | SEQ ID NO. 186 | SEQ ID NO. 187 | SEQ ID NO. 188 |
| VH43 | SEQ ID NO. 189 | SEQ ID NO. 190 | SEQ ID NO. 191 |
| VH44 | SEQ ID NO. 192 | SEQ ID NO. 193 | SEQ ID NO. 194 |
| VH45 | SEQ ID NO. 195 | SEQ ID NO. 196 | SEQ ID NO. 197 |
| VH46 | SEQ ID NO. 198 | SEQ ID NO. 199 | SEQ ID NO. 200 |
| VH47 | SEQ ID NO. 201 | SEQ ID NO. 202 | SEQ ID NO. 203 |
| VH48 | SEQ ID NO. 204 | SEQ ID NO. 205 | SEQ ID NO. 206 |
| VH49 | SEQ ID NO. 207 | SEQ ID NO. 208 | SEQ ID NO. 209 |
| VH50 | SEQ ID NO. 210 | SEQ ID NO. 211 | SEQ ID NO. 212 |
| VH51 | SEQ ID NO. 213 | SEQ ID NO. 214 | SEQ ID NO. 215 |
| VH52 | SEQ ID NO. 216 | SEQ ID NO. 217 | SEQ ID NO. 218 |
| VH53 | SEQ ID NO. 219 | SEQ ID NO. 220 | SEQ ID NO. 221 |
| VH54 | SEQ ID NO. 222 | SEQ ID NO. 223 | SEQ ID NO. 224 |
| VH55 | SEQ ID NO. 225 | SEQ ID NO. 226 | SEQ ID NO. 227 |
| VH56 | SEQ ID NO. 228 | SEQ ID NO. 229 | SEQ ID NO. 230 |
| VH57 | SEQ ID NO. 231 | SEQ ID NO. 232 | SEQ ID NO. 233 |
| VH58 | SEQ ID NO. 234 | SEQ ID NO. 235 | SEQ ID NO. 236 |
| VH59 | SEQ ID NO. 237 | SEQ ID NO. 238 | SEQ ID NO. 239 |
| VH60 | SEQ ID NO. 240 | SEQ ID NO. 241 | SEQ ID NO. 242 |
| VH61 | SEQ ID NO. 243 | SEQ ID NO. 244 | SEQ ID NO. 245 |
| VH62 | SEQ ID NO. 246 | SEQ ID NO. 247 | SEQ ID NO. 248 |
| VH63 | SEQ ID NO. 249 | SEQ ID NO. 250 | SEQ ID NO. 251 |
| VH64 | SEQ ID NO. 252 | SEQ ID NO. 253 | SEQ ID NO. 254 |
| VH65 | SEQ ID NO. 255 | SEQ ID NO. 256 | SEQ ID NO. 257 |
| VH66 | SEQ ID NO. 258 | SEQ ID NO. 259 | SEQ ID NO. 260 |

each CDR1, CDR2 and CDR3 is coded according to the prevailing analysis methods of KABAT, Chothia or IMGT; preferably, the substitution is a conservative amino acid substitution.

In particular, for example, the nanobody or the antigen-binding fragment of the invention, wherein:
(1) the CDR1, CDR2 and CDR3 have sequences as shown in SEQ ID NO. 63, 64 and 65, respectively;
(2) the CDR1, CDR2 and CDR3 have sequences as shown in SEQ ID NO. 66, 67 and 68, respectively;
(3) the CDR1, CDR2 and CDR3 have sequences as shown in SEQ ID NO. 69, 70 and 71, respectively;
(4) the CDR1, CDR2 and CDR3 have sequences as shown in SEQ ID NO. 72, 73 and 74, respectively;
(5) the CDR1, CDR2 and CDR3 have sequences as shown in SEQ ID NO. 75, 76 and 77, respectively;
(6) the CDR1, CDR2 and CDR3 have sequences as shown in SEQ ID NO. 78, 79 and 80, respectively;
(7) the CDR1, CDR2 and CDR3 have sequences as shown in SEQ ID NO. 81, 82 and 83, respectively;
(8) the CDR1, CDR2 and CDR3 have sequences as shown in SEQ ID NO. 84, 85 and 86, respectively;
(9) the CDR1, CDR2 and CDR3 have sequences as shown in SEQ ID NO. 87, 88 and 89, respectively;
(10) the CDR1, CDR2 and CDR3 have sequences as shown in SEQ ID NO. 90, 91 and 92, respectively;
(11) the CDR1, CDR2 and CDR3 have sequences as shown in SEQ ID NO. 93, 94 and 95, respectively;
(12) the CDR1, CDR2 and CDR3 have sequences as shown in SEQ ID NO. 96, 97 and 98, respectively;
(13) the CDR1, CDR2 and CDR3 have sequences as shown in SEQ ID NO. 99, 100 and 101, respectively;
(14) the CDR1, CDR2 and CDR3 have sequences as shown in SEQ ID NO. 102, 103 and 104, respectively;
(15) the CDR1, CDR2 and CDR3 have sequences as shown in SEQ ID NO. 105, 106 and 107, respectively;
(16) the CDR1, CDR2 and CDR3 have sequences as shown in SEQ ID NO. 108, 109 and 110, respectively;
(17) the CDR1, CDR2 and CDR3 have sequences as shown in SEQ ID NO.

111, 112 and 113, respectively;
(18) the CDR1, CDR2 and CDR3 have sequences as shown in SEQ ID NO. 114, 115 and 116, respectively;
(19) the CDR1, CDR2 and CDR3 have sequences as shown in SEQ ID NO. 117, 118 and 119, respectively;
(20) the CDR1, CDR2 and CDR3 have sequences as shown in SEQ ID NO. 120, 121 and 122, respectively;
(21) the CDR1, CDR2 and CDR3 have sequences as shown in SEQ ID NO. 123, 124 and 125, respectively;
(22) the CDR1, CDR2 and CDR3 have sequences as shown in SEQ ID NO. 126, 127 and 128, respectively;
(23) the CDR1, CDR2 and CDR3 have sequences as shown in SEQ ID NO. 129, 130 and 131, respectively;
(24) the CDR1, CDR2 and CDR3 have sequences as shown in SEQ ID NO. 132, 133 and 134, respectively;
(25) the CDR1, CDR2 and CDR3 have sequences as shown in SEQ ID NO. 135, 136 and 137, respectively;
(26) the CDR1, CDR2 and CDR3 have sequences as shown in SEQ ID NO. 138, 139 and 140, respectively;
(27) the CDR1, CDR2 and CDR3 have sequences as shown in SEQ ID NO. 141, 142 and 143, respectively;
(28) the CDR1, CDR2 and CDR3 have sequences as shown in SEQ ID NO. 144, 145 and 146, respectively;
(29) the CDR1, CDR2 and CDR3 have sequences as shown in SEQ ID NO. 147, 148 and 149, respectively;
(30) the CDR1, CDR2 and CDR3 have sequences as shown in SEQ ID NO. 150, 151 and 152, respectively;
(31) the CDR1, CDR2 and CDR3 have sequences as shown in SEQ ID NO. 153, 154 and 155, respectively;
(32) the CDR1, CDR2 and CDR3 have sequences as shown in SEQ ID NO. 156, 157 and 158, respectively;
(33) the CDR1, CDR2 and CDR3 have sequences as shown in SEQ ID NO. 159, 160 and 161, respectively;
(34) the CDR1, CDR2 and CDR3 have sequences as shown in SEQ ID NO. 162, 163 and 164, respectively;
(35) the CDR1, CDR2 and CDR3 have sequences as shown in SEQ ID NO. 165, 166 and 167, respectively;
(36) the CDR1, CDR2 and CDR3 have sequences as shown in SEQ ID NO. 168, 169 and 170, respectively;
(37) the CDR1, CDR2 and CDR3 have sequences as shown in SEQ ID NO. 171, 172 and 173, respectively;
(38) the CDR1, CDR2 and CDR3 have sequences as shown in SEQ ID NO. 174, 175 and 176, respectively;
(39) the CDR1, CDR2 and CDR3 have sequences as shown in SEQ ID NO. 177, 178 and 179, respectively;
(40) the CDR1, CDR2 and CDR3 have sequences as shown in SEQ ID NO. 180, 181 and 182, respectively;
(41) the CDR1, CDR2 and CDR3 have sequences as shown in SEQ ID NO. 183, 184 and 185, respectively;
(42) the CDR1, CDR2 and CDR3 have sequences as shown in SEQ ID NO. 186, 187 and 188, respectively;
(43) the CDR1, CDR2 and CDR3 have sequences as shown in SEQ ID NO. 189, 190 and 191, respectively;
(44) the CDR1, CDR2 and CDR3 have sequences as shown in SEQ ID NO. 192, 193 and 194, respectively;
(45) the CDR1, CDR2 and CDR3 have sequences as shown in SEQ ID NO. 195, 196 and 197, respectively;
(46) the CDR1, CDR2 and CDR3 have sequences as shown in SEQ ID NO. 198, 199 and 200, respectively;
(47) the CDR1, CDR2 and CDR3 have sequences as shown in SEQ ID NO. 201, 202 and 203, respectively;
(48) the CDR1, CDR2 and CDR3 have sequences as shown in SEQ ID NO. 204, 205 and 206, respectively;
(49) the CDR1, CDR2 and CDR3 have sequences as shown in SEQ ID NO. 207, 208 and 209, respectively;
(50) the CDR1, CDR2 and CDR3 have sequences as shown in SEQ ID NO. 210, 211 and 212, respectively;
(51) the CDR1, CDR2 and CDR3 have sequences as shown in SEQ ID NO. 213, 214 and 215, respectively;
(52) the CDR1, CDR2 and CDR3 have sequences as shown in SEQ ID NO. 216, 217 and 218, respectively;
(53) the CDR1, CDR2 and CDR3 have sequences as shown in SEQ ID NO. 219, 220 and 221, respectively;
(54) the CDR1, CDR2 and CDR3 have sequences as shown in SEQ ID NO. 222, 223 and 224, respectively;
(55) the CDR1, CDR2 and CDR3 have sequences as shown in SEQ ID NO. 225, 226 and 227, respectively;
(56) the CDR1, CDR2 and CDR3 have sequences as shown in SEQ ID NO. 228, 229 and 230, respectively;
(57) the CDR1, CDR2 and CDR3 have sequences as shown in SEQ ID NO. 231, 232 and 233, respectively;
(58) the CDR1, CDR2 and CDR3 have sequences as shown in SEQ ID NO. 234, 235 and 236, respectively;
(59) the CDR1, CDR2 and CDR3 have sequences as shown in SEQ ID NO. 237, 238 and 239, respectively;
(60) the CDR1, CDR2 and CDR3 have sequences as shown in SEQ ID NO. 240, 241 and 242, respectively;
(61) the CDR1, CDR2 and CDR3 have sequences as shown in SEQ ID NO. 243, 244 and 245, respectively;
(62) the CDR1, CDR2 and CDR3 have sequences as shown in SEQ ID NO. 246, 247 and 248, respectively;
(63) the CDR1, CDR2 and CDR3 have sequences as shown in SEQ ID NO. 249, 250 and 251, respectively;
(64) the CDR1, CDR2 and CDR3 have sequences as shown in SEQ ID NO. 252, 253 and 254, respectively;
(65) the CDR1, CDR2 and CDR3 have sequences as shown in SEQ ID NO. 255, 256 and 257, respectively;
(66) the CDR1, CDR2 and CDR3 have sequences as shown in SEQ ID NO. 258, 259 and 260, respectively; or, the CDR1, CDR2 and CDR3 have a sequence combination having 1, 2, 3 or more amino acid insertions, deletions and/or substitutions compared with the above sequence combinations (1)-(66); it is preferably a substitution, more preferably a substitution of a conservative amino acid residue.

In another specific embodiment, the invention provides an antibody or an antigen-binding fragment thereof comprising:
(1) a variable region having a sequence as shown in SEQ ID NO: 19, 21, 23, 25, 27, 29, 31, 33, 35, 37, 39, 41, 43, 45, 47, 49, 51, 53, 55, 57, 59 or 61;
(2) an amino acid sequence having at least 90% identity, preferably at least 91%, 92%, 93%, 94%, 95%, 96%, 97%, 98% or 99% identity with the sequence shown in (1) above; or,
(3) the framework region of the nanobody or the antigen-binding fragment has at least 90% identity, preferably at least 91%, 92%, 93%, 94%, 95%, 96%, 97%, 98%, or 99% identity with the framework region of the amino acid sequence as shown in SEQ ID NO: 19, 21, 23, 25, 27, 29, 31, 33, 35, 37, 39, 41, 43, 45, 47, 49, 51, 53, 55, 57, 59 or 61.

In a preferred embodiment, the antibody or the antigen-binding fragment thereof of the present invention binds to human EGFR and EGFRvIII with a dissociation constant (KD) of no more than 10⁻⁷ nM, and binds to cynomolgus monkey EGFR with a dissociation constant (KD) of no more than 10⁻⁸ nM;

Optionally, the nanobody or the antigen-binding fragment binds to or does not bind to monkey EGFR protein;

Optionally, the nanobody or the antigen-binding fragment binds to or does not bind to murine EGFR protein;

optionally, the nanobody or the antigen-binding fragment does not compete with antibody C225 or antibody 7D12.

In a preferred embodiment, the antibody or the antigen-binding fragment thereof of the present invention comprises a sequence of the constant region of any one of human or murine antibody IgG1, IgG2, IgG3, IgG4, IgA, IgM, IgE or IgD; preferably, a sequence comprising the constant region of human or murine antibody IgG1, IgG2, IgG3 or IgG4.

In a preferred embodiment, the antibody or the antigen-binding fragment thereof of the invention further comprises a heavy chain constant region sequence without a CH1 fragment.

In a preferred embodiment, the antibody or the antigen-binding fragment thereof of the present invention further comprises a heavy chain constant region sequence with CH2 and CH3 fragments, or, the antibody or the antigen-binding fragment further comprises an antibody Fc region;

the antibody constant region or the antibody Fc region is linked to the antibody or the antigen-binding fragment with or without a linker peptide;

optionally, the antibody constant region or the antibody Fc region is derived from camelidae, mice, rats, rabbits, sheep or humans;

optionally, the antibody constant region or the antibody Fc region is derived from IgG, IgA, IgM, IgD or IgE.

In a preferred embodiment, the antibody or the antigen-binding fragment thereof of the invention is chimeric or humanized or fully human; preferably, the antibody or the antigen-binding fragment is selected from a monoclonal antibody, a polyclonal antibody, a natural antibody, an engineered antibody, a monospecific antibody, a multispecific antibody (for example, a bispecific antibody), a monovalent antibody, a multivalent antibody, a full-length antibody, an antibody fragment, a naked antibody, a conjugated antibody, a humanized antibody, a fully human antibody, Fab, Fab′, F(ab′)2, Fd, Fv, scFv, a diabody or a single domain antibody.

In a preferred embodiment, the antibody or the antigen-binding fragment thereof of the invention is further coupled with a therapeutic agent or a tracer; preferably, the therapeutic agent is selected from a radioisotope, a chemotherapeutic agent or an immunomodulator, and the tracer is selected from a radiological contrast agent, a paramagnetic ion, a metal, a fluorescent label, a chemiluminescence label, an ultrasound contrast agent or a photosensitizer.

In a preferred embodiment, the present invention also provides a multispecific antigen-binding molecule; preferably, the multispecific antigen-binding molecule comprises a first antigen-binding module and a second antigen-binding module, the first antigen-binding module comprises the antibody or the antigen-binding fragment described in any one of the above, the second antigen-binding module specifically binds to other antigens than EGFR or binds to a different EGFR epitope than the first antigen-binding module;

preferably, the other antigens are selected from CD3, PD-1, PD-L1, Her2, EpCAM, CD16, CD20, CD30, CD33, CD47, CD52, CD64, CD133, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, or FAP;

preferably, the multispecific antibody is “bispecific”, “trispecific” or “tetraspecific”.

In a preferred embodiment, the present invention provides a chimeric antigen receptor (CAR); preferably, the chimeric antigen receptor at least comprises an extracellular antigen-binding domain, a transmembrane domain and an intracellular signaling domain, and the extracellular antigen-binding domain comprises any of the EGFR antibody or the antigen-binding fragment described above.

In a preferred embodiment, the present invention provides an immune effector cell; preferably, the immune effector cell comprises the chimeric antigen receptor described above or a nucleic acid fragment encoding the chimeric antigen receptor described above;

preferably, the immune effector cell is selected from a T cell, a NK cell (a natural killer cell), a NKT cell (a natural killer cell), a monocyte, a macrophage, a dendritic cell or a mast cell; the T cell may be selected from an inflammatory T cell, a cytotoxic T cell, a regulatory T cell (Treg) or a helper T cell;

preferably, the immune effector cell is an allogeneic immune effector cell or an autologous immune cell.

In a preferred embodiment, the present invention provides an isolated nucleic acid molecule encoding the nanobody, the antigen-binding fragment, or any combination thereof according to any one of the above aspects of the present invention, the multispecific antigen-binding molecule described above or the chimeric antigen receptor described above.

In some embodiments, the present invention provides an expression vector comprising the isolated nucleic acid molecule of the present invention described above.

In some embodiments, the present invention provides a host cell comprising the isolated nucleic acid molecule or the expression vector of the present invention described above.

In a preferred embodiment, the host cell is a eukaryotic cell or a prokaryotic cell; more preferably, the host cell is derived from a mammalian cell, a yeast cell, an insect cell, Escherichia coli and/or Bacillus subtilis; more preferably, the host cell is selected from HEK293E or Chinese hamster ovary (CHO) cell.

In some embodiments, the present invention provides a method for preparing an antibody or an antigen-binding fragment or a multispecific antigen-binding molecule, the method comprises culturing the host cell of the present invention described above under appropriate conditions, and isolating the antibody or the antigen-binding fragment or the multispecific antigen-binding molecule.

In some embodiments, the present invention provides a method for preparing an immune effector cell, wherein the CAR nucleic acid fragment described above is introduced into the immune effector cell, preferably, the method further comprises enabling the immune effector cell to express the CAR described above.

In some embodiments, the present invention provides a pharmaceutical composition comprising the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, or a product (e.g., an antibody and an antigen-binding fragment) prepared by the method of the invention described above, and a pharmaceutically acceptable carrier.

In a preferred embodiment, the pharmaceutical composition further comprises a pharmaceutically acceptable carrier, diluent or adjuvant; more preferably, the pharmaceutical composition further comprises an additional antineoplastic agent.

In some embodiments, the present invention provides a method for preventing and/or treating a tumor disease or an inflammatory disease, the method comprises administering the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above to a patient in need thereof. The tumor disease or the inflammatory disease is a tumor disease or an inflammatory disease with EGFR overexpression. The tumor disease is preferably glioma, melanoma, glioblastoma, sarcoma, brain tumor, non-small cell lung cancer, bladder cancer, breast cancer, endometrial cancer, lung cancer, ovarian cancer, prostate cancer, colon cancer, stomach cancer, liver cancer, kidney cancer, brain cancer, laryngeal cancer, rectal cancer, pancreatic cancer, head and neck cancer, esophageal adenocarcinoma, esophageal squamous cell carcinoma, solid tumors, non-Hodgkin's lymphoma, thyroid cancer, nasopharyngeal carcinoma, esophageal carcinoma, or skin cancers; the inflammatory disease is preferably inflammatory arthritis, serpedo, psoriasis, rheumatoid arthritis, spondylarthropathies, contact dermatitis, delayed hypersensitivity reaction, endometriosis, scar formation, benign prostatic hyperplasia, eczema, dermatitis, nerve inflammation, liver diseases and nephritis, gastrointestinal diseases, inflammatory bowel diseases, Crohn's disease or gastritis.

In some embodiments, the present invention provides the use of the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above in the preparation of a drug for preventing and/or treating a tumor disease or an inflammatory disease. The tumor disease is preferably glioma, melanoma, glioblastoma, sarcoma, brain tumor, non-small cell lung cancer, bladder cancer, breast cancer, endometrial cancer, lung cancer, ovarian cancer, prostate cancer, colon cancer, stomach cancer, liver cancer, kidney cancer, brain cancer, laryngeal cancer, rectal cancer, pancreatic cancer, head and neck cancer, esophageal adenocarcinoma, esophageal squamous cell carcinoma, solid tumors, non-Hodgkin's lymphoma, thyroid cancer, nasopharyngeal carcinoma, esophageal carcinoma, or skin cancers; the inflammatory disease is preferably inflammatory arthritis, serpedo, psoriasis, rheumatoid arthritis, spondylarthropathies, contact dermatitis, delayed hypersensitivity reaction, endometriosis, scar formation, benign prostatic hyperplasia, eczema, dermatitis, nerve inflammation, liver diseases and nephritis, gastrointestinal diseases, inflammatory bowel diseases, Crohn's disease or gastritis.

In some embodiments, the present invention provides the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, a product (e.g., an antibody and an antigen-binding fragment) prepared by the method of the invention described above, or the pharmaceutical composition of the invention described above for preventing and/or treating a tumor disease or an inflammatory disease. The tumor disease is preferably glioma, melanoma, glioblastoma, sarcoma, brain tumor, non-small cell lung cancer, bladder cancer, breast cancer, endometrial cancer, lung cancer, ovarian cancer, prostate cancer, colon cancer, stomach cancer, liver cancer, kidney cancer, brain cancer, laryngeal cancer, rectal cancer, pancreatic cancer, head and neck cancer, esophageal adenocarcinoma, esophageal squamous cell carcinoma, solid tumors, non-Hodgkin's lymphoma, thyroid cancer, nasopharyngeal carcinoma, esophageal carcinoma, or skin cancers; the inflammatory disease is preferably inflammatory arthritis, serpedo, psoriasis, rheumatoid arthritis, spondylarthropathies, contact dermatitis, delayed hypersensitivity reaction, endometriosis, scar formation, benign prostatic hyperplasia, eczema, dermatitis, nerve inflammation, liver diseases and nephritis, gastrointestinal diseases, inflammatory bowel diseases, Crohn's disease or gastritis.

In some embodiments, the present invention provides a kit comprising the antibody or the antigen-binding fragment of the present invention described above, the multispecific antigen-binding molecule of the present invention described above, the chimeric antigen receptor of the present invention described above, the immune effector cell of the present invention described above, the isolated nucleic acid molecule of the present invention described above, the expression vector of the present invention described above, the cell of the present invention described above, or a product (e.g., an antibody and an antigen-binding fragment) prepared by the method of the invention described above, or the pharmaceutical composition of the invention described above, and instructions for use.

### Definition and Description of Terminology

Unless otherwise specified, the terms used herein have the meanings commonly understood by those of ordinary skill in the art. For a term that is explicitly defined herein, the meaning of the term is to be determined based on the definition.

As used herein, the term “antibody” (Ab) refers to an immunoglobulin molecule that specifically binds to or is immunoreactive with an antigen of interest and includes polyclonal, monoclonal, genetically engineered, and other modified forms of an antibody (including but not limited to a chimeric antibody, a humanized antibody, a fully human antibody, a heteroconjugate antibody (e.g. a bispecific, trispecific and tetraspecific antibody, a diabody, a triabody and a tetrabody)), an antibody conjugate and an antigen-binding fragment of an antibody (including, for example, Fab′, F(ab′)2, Fab, Fv, rIgG and scFv fragment). Furthermore, unless otherwise indicated, the term “monoclonal antibody” (mAb) is intended to include both an intact antibody molecule and an incomplete antibody fragment (such as Fab and F(ab′)2 fragment, which lack the Fc fragment of the intact antibody (cleared more quickly from circulation in animals), thus lacking Fc-mediated effector function) capable of specifically binding to a target protein (see Wahl et al., J. Nucl. Med. 24:316, 1983; the content of which is incorporated herein by reference).

An “antibody” herein may be derived from any animal, including but not limited to humans and non-human animals selected from primates, mammals, rodents and vertebrates, such as camelids, llamas, guanaco, alpaca, sheep, rabbits, mice, rats or cartilaginous fishes (such as sharks).

The term “natural antibody” herein refers to an antibody produced and paired by the immune system of a multicellular organism. The term “engineered antibody” herein refers to a non-natural antibody obtained through genetic engineering, antibody engineering, etc. For example, “engineered antibody” includes a humanized antibody, a small molecule antibody (such as scFv, etc.), a bispecific antibody, etc.

The term “monospecific” herein refers to having one or more binding sites, wherein each binding site binds the same epitope of the same antigen.

The term “multispecific” herein refers to having at least two antigen binding sites, each of which binds a different epitope of the same antigen or a different epitope of a different antigen. Thus, terms such as “bispecific”, “trispecific”, “tetraspecific” and the like refer to the number of different epitopes to which an antibody/an antigen-binding molecule can bind.

The term “valence” herein refers to the presence of a defined number of binding sites in an antibody/an antigen-binding molecule. Thus, the terms “monovalent”, “bivalent”, “tetravalent” and “hexavalent” refer to the presence of one binding site, two binding sites, four binding site and six binding sites in an antibody/an antigen-binding molecule, respectively.

“Full-length antibody”, “complete antibody” and “intact antibody” are used interchangeably herein to mean that they have a structure substantially similar to that of a natural antibody.

As used herein, the term “antigen-binding fragment” refers to one or more antibody fragments that retain the ability to specifically bind to a target antigen. The antigen-binding function of an antibody can be performed by a fragment of a full-length antibody. The antibody fragment can be Fab, F(ab′)2, scFv, SMIP, a diabody, a triabody, an affibody, a nanobody, an aptamer or a domain antibody. Examples of a binding fragment encompassed by the term “antigen-binding fragment” of an antibody include, but are not limited to: (i) an Fab fragment, which is a monovalent fragment consisting of VL, VH, CL and CH1 domains; (ii) an F(ab)2 fragment, which is a bivalent fragment comprising two Fab fragments connected by a disulfide bond in the hinge region; (iii) an Fd fragment consisting of VH and CH1 domains; (iv) an FAT fragment consisting of the VL and VH domains of a single arm of an antibody; (V) dAb comprising VH and VL domains; (vi) a dAb fragment consisting of a VH domain (Ward et al., Nature 341:544-546, 1989); (vii) dAb consisting of VH or VL domains; (viii) an isolated complementarity determining region (CDR); and (ix) a combination of two or more isolated CDRs, the CDRs may optionally be linked by a synthetic linker. Furthermore, although the two domains VL and VH of the Fv fragment are encoded by separate genes, these two domains can be joined using a recombinant method through a linker that enables forming a single protein chain (referred to as a single chain Fv (scFv); see e.g., Bird et al., Science 242:423-426, 1988 and Huston et al., Proc. Natl. Acad. Sci. USA 85:5879-5883, 1988) in which the VL and VH regions pair to form a monovalent molecule. These antibody fragments can be obtained using conventional techniques known to those skilled in the art, and these fragments are screened for use in the same manner as an intact antibody. The antigen-binding fragment can be produced by recombinant DNA techniques, enzymatic or chemical cleavage of an intact immunoglobulin, or in some embodiments by chemical peptide synthesis procedures known in the art.

As used herein, the term “EGFR” refers to members of the epidermal growth factor receptor family (EGFRs), the members of the EGFR family include: EGFR (ErbB1), HER2 (ErbB2), HER3 (ErbB3) and HER4 (ErbB4). Epidermal growth factor receptor (EGFR) is a member of the ErbB family of transmembrane tyrosine kinase receptors. The ligands of EGFR are EGF and transforming growth factor-alpha (TGF-α). The ligand binds to EGFR and induces conformational change and the formation of a dimer, leading to the activation of intracellular tyrosine kinases (TKs), and the subsequent enzymatic cascade reactions lead to tumor cell proliferation, invasion, metastasis, neovascularization and reduced programmed death.

As used herein, the term “EGFRvIII” refers to the type III EGF deletion mutant receptor, characterized by a deletion of exon 2-7 in the EGFR mRNA. These deletions correspond to cDNA nucleotides 275-1075 (encoding amino acids 6-276) and are presumably caused by alternative splicing or rearrangement. The 801 bp deletion in the extracellular domain of the EGFR gene caused an in-frame truncation of the normal EGFR protein, resulting in a 145 kDa receptor, thereby forming a tumor-specific immunogenic epitope. EGFRvIII expression has been observed in many tumor types, including glioblastoma multiforme (GBM), but is rare in normal tissues.

As used herein, the term “bispecific antibody” refers to an antibody, typically a human or humanized antibody, that has monoclonal binding specificities for at least two different antigens. In the present invention, one of the binding specificities can be detected against an antigen epitope of EGFR, and the other can be detected against another antigen epitope of EGFR or any other antigen except EGFR, such as a cell surface protein, a receptor, a receptor subunit, a tissue-specific antigen, a viral-derived protein, a viral-encoded envelope protein, a bacterial-derived protein, or a bacterial surface protein.

As used herein, the term “chimeric” antibody refers to an antibody that has a variable sequence derived from an immunoglobulin of one organism (such as a rat or mouse) and a constant region derived from an immunoglobulin of a different organism, such as human. Methods for producing the chimeric antibody are known in the art. See, e.g., Morrison, 1985, Science 229(4719): 1202-7; Oi et al., 1986, Bio Techniques 4: 214-221; Gillies et al., 1985 J Immunol Methods 125: 191-202; incorporated herein by reference.

As used herein, the term “heavy chain antibody” refers to an antibody that lacks the light chains of a conventional antibody. The term specifically includes, but is not limited to, a homodimeric antibody comprising a VH antigen binding domain and CH2 and CH3 constant domains in the absence of a CH1 domain.

As used herein, the term “nanobody” refers to a natural heavy chain antibody without light chains in camel and cloning its variable region can obtain a single domain antibody, also known as VHH (Variable domain of heavy chain of heavy chain antibody), which only consists of a heavy chain variable region, and is the smallest functional antigen-binding fragment. For a further description of VHH and nanobody, reference is made to the review article by Muyldermans (2001, Reviews in Molecular Biotechnology 74: 277-302), and to the following patent applications mentioned as general background art: WO 94/04678, WO 95/04079 and WO 96/34103 of the Free University of Brussels; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of Vlaams Instituut voor Biotechnologie (VIB); WO 03/050531 of Algonomics N.V. and Ablynx N.V.; WO 01/90190 of the National Research Council of Canada; WO 03/025020 (=EP 1433793) of Institute of Antibodies; and WO 04/041867, WO 04/041862, WO 04/041865, WO 04/041863, WO 04/062551, WO 05/044858, WO 06/40153, WO 06/079372, WO 06/122786, WO 06/122787 and WO 06/122825 of Ablynx N. V. and further published patent applications of Ablynx N. V. Reference is also made to the additional prior art mentioned in these applications, in particular the list of references mentioned on pages 41-43 of International Application WO 06/040153, which list and references are incorporated herein by reference. As described in these references, nanobody (in particular VHH sequences and partially humanized nanobody) may inter alia be characterized by the presence of one or more “signature residues” in one or more framework sequences. Further descriptions of nanobody can be found in, for example, WO 08/101985 and WO 08/142164, including humanization and/or camelization of nanobody, as well as other modifications, parts or fragments, derivatives or “nanobody fusion”, multivalent constructs (including some non-limiting examples of linker sequences) and various modifications that increase the half-life of a nanobody and a formulation thereof. For a further general description of nanobody, reference is made to the prior art cited herein, for example WO 08/020079 (page 16).

As used herein, the term “complementarity determining region” (CDR) refers to hypervariable regions found in both light and heavy chain variable domains. The more conserved portions in variable domains are called the framework regions (FR). As understood in the art, the amino acid positions representing the hypervariable regions of an antibody can vary according to the context and various definitions known in the art. Some positions within variable domains can be considered heterozygous hypervariable positions because these positions can be considered to be within the hypervariable regions under one set of criteria (such as IMGT or KABAT) but outside the hypervariable regions under a different set of criteria (such as KABAT or IMGT). One or more of these positions may also be found in extended hypervariable regions. The invention includes an antibody comprising modifications in these hybrid hypervariable positions. The variable domains of the native heavy and light chains respectively comprise four framework regions that largely adopt a sheet configuration and connected by three CDRs (CDR1, CDR2, and CDR3) that form loops connecting the sheets, and in some cases form part of the sheet structure. The CDRs in each chain are held tightly together by the FR regions in the order of FR1-CDR1-FR2-CDR2-FR3-CDR3-FR4, and together with CDRs from other antibody chains contribute to the formation of the antibody's antigen-binding site (see Kabat et al., Sequences of Proteins of Immunological Interest, National Institute of Health, Bethesda, Md. 1987; which is incorporated herein by reference). For example, herein, CDR1-VH, CDR2-VH, and CDR3-VH refer to the first CDR, the second CDR, and the third CDR of the heavy chain variable region (VH), respectively, and these three CDRs constitute the CDR combination (VHCDR combination) of the heavy chain (or its variable region); CDR1-VL, CDR2-VL, and CDR3-VL refer to the first CDR, the second CDR, and the third CDR of the light chain variable region (VL), respectively, and these three CDRs constitute the CDR combination (VLCDR combination) of the light chain (or its variable region).

As used herein, the term “monoclonal antibody” refers to an antibody derived from a single clone (including any eukaryotic, prokaryotic, or phage clone), without limitation by the method by which the antibody is produced.

As used herein, the term “VH” refers to the variable region of an immunoglobulin heavy chain of an antibody, including the heavy chain of an Fv, scFv or Fab. The term “VL” refers to the variable region of an immunoglobulin light chain, including the light chain of an Fv, scFv, dsFv or Fab.

The term “heavy chain constant region” herein refers to the carboxy-terminal portion of the heavy chain of an antibody, which is not directly involved in the binding of the antibody to an antigen, but exhibits effector functions, such as interaction with Fc receptors, and has a more conserved amino acid sequence relative to the antibody's variable domain. A “heavy chain constant region” comprises at least one of the following: a CH1 domain, a hinge region, a CH2 domain, a CH3 domain, or a variant or fragment thereof “Heavy chain constant region” includes “full-length heavy chain constant region” and “heavy chain constant region fragment”, the former has a structure substantially similar to that of a natural antibody constant region, while the latter only includes “a part of the full-length heavy chain constant region”. For example, a typical “full-length antibody heavy chain constant region” consists of CH1 domain-hinge region-CH2 domain-CH3 domain; when the antibody is IgE, it also includes a CH4 domain; when the antibody is a heavy chain antibody, it does not include the CH1 domain. For example, a typical “heavy chain constant region fragment” can be selected from CH1, Fc or CH3 domains.

The term “light chain constant region” herein refers to the carboxy-terminal portion of the light chain of an antibody, which is not directly involved in the binding of the antibody to an antigen. The light chain constant region may be selected from a constant κ domain or a constant λ domain.

The term “Fc” herein refers to the carboxy-terminal portion of an antibody obtained by papain hydrolysis of the intact antibody, which typically includes the CH3 and CH2 domains of the antibody. Fc region includes, for example, a native sequence Fc region, a recombinant Fc region and a variant Fc region. Although the boundary of the Fc region of an immunoglobulin heavy chain can vary slightly, the Fc region of a human IgG heavy chain is generally defined as extending from the amino acid residue at position Cys226 or from Pro230 to the carboxyl terminus. The C-terminal lysine of the Fc region (residue 447 according to the EU numbering system) may be removed, for example, during the production or purification of the antibody, or by recombinant engineering of the nucleic acid encoding the heavy chain of the antibody, thus the Fc region may comprise or may not comprise Lys447.

The term “humanized antibody” herein refers to a genetically engineered non-human antibody whose amino acid sequence has been modified to increase sequence homology with a human antibody. Generally speaking, all or part of the CDR regions of a humanized antibody are derived from a non-human antibody (donor antibody), and all or part of the non-CDR regions (for example, variable region FR and/or constant region) are derived from human Immunoglobulin (recipient antibody). Humanized antibody usually retains or partially retains the expected properties of the donor antibody, including but not limited to, antigen specificity, affinity, reactivity, ability to enhance immune cell activity, ability to enhance immune response, etc.

The term “fully human antibody” herein refers to an antibody having variable regions in which both the FRs and CDRs are derived from human germline immunoglobulin sequences. Furthermore, if the antibody comprises a constant region, the constant region also is derived from human germline immunoglobulin sequences. Fully human antibody herein may include amino acid residues not encoded by human germline immunoglobulin sequences (for example, mutations introduced by random or site-specific mutagenesis in vitro or by somatic mutation in vivo). However, a “fully human antibody” herein is not intended to include an antibody in which CDR sequences derived from the germline of another mammalian species (for example, mouse) have been grafted onto human framework sequences.

The term “naked antibody” herein refers to an antibody that is not linked, fused or conjugated to another agent or molecule (for example, label or drug), peptide or polypeptide. In specific embodiments, the naked antibody expressed by a mammalian host cell can be glycosylated by the host cell's glycosylation machinery (for example, glycosylase). In certain embodiments, the naked antibody is not glycosylated when expressed by a host cell that does not have its own glycosylation machinery (for example, glycosylase). In certain embodiments, the naked antibody is an intact antibody, while in other embodiments, the naked antibody is the antigen-binding fragment of an intact antibody, such as Fab antibody.

The term “conjugated antibody” herein refers to an antibody that can be associated with a pharmaceutically acceptable carrier or diluent and can be a monoclonal antibody, a chimeric antibody, a humanized antibody, or a human antibody.

The term “diabody” herein refers to bivalent bispecific antibody that can bind to different epitopes on the same or different antigens.

As used herein, the term “percent (%) sequence identity” refers to the percentage of amino acid (or nucleotide) residues of the candidate sequence that are identical to those of the reference sequence after aligning the sequences and introducing gaps (if necessary) in order to achieve maximum percentage sequence identity (for example, for optimal alignment, gaps can be introduced in one or both of the candidate sequence and the reference sequence, and non-homologous sequences can be ignored for comparison purpose). For purpose of determining percent sequence identity, alignment can be achieved in a variety of ways well known to those skilled in the art, for example, using publicly available computer software such as BLAST, ALIGN or Megalign (DNASTAIi) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence shows sequence identity from 50% to 100% in the full length of the candidate sequence or in the selected part of the continuous amino acid (or nucleotide) residues of the candidate sequence. The length of the candidate sequence aligned for comparison purpose is at least 30% (e.g., 30%, 40%, 50%, 60%, 70%, 80%, 90% or 100%) of the length of the reference sequence. When a position in the candidate sequence is occupied by the same amino acid (or nucleotide) residue as the corresponding position in the reference sequence, then the molecules are identical at that position.

The term “conservative amino acid” herein generally refers to amino acids that belong to the same class or have similar characteristics (for example, charge, side chain size, hydrophobicity, hydrophilicity, backbone conformation, and rigidity). For example, the amino acids in each of the following groups belong to each other's conservative amino acid residues, and the substitution of amino acid residues in the group belongs to the conservative amino acid substitution:
(1) acidic amino acids: Asp(D) and Glu(E);
(2) basic amino acids: Lys (K), Arg (R) and His (H);
(3) hydrophilic uncharged amino acids: Ser (S), Thr (T), Asn (N) and Gln (Q);
(4) Aliphatic uncharged amino acids: Gly (G), Ala (A), Val (V), Leu (L) and Ile (I);
(5) nonpolar uncharged amino acids: Cys (C), Met (M) and Pro (P);
(6) aromatic amino acids: Phe (F), Tyr (Y) and Trp (W).

The term “Kabat numbering system” herein generally refers to the immunoglobulin alignment and numbering system proposed by Elvin A. Kabat (see, for example, Kabat et al., Sequences of Proteins of Immunological Interest, 5th Ed. Public Health Service, National Institutes of Health, Bethesda, Md., 1991).

The term “Chothia numbering system” herein generally refers to the immunoglobulin numbering system proposed by Chothia et al., which is a classical rule for identifying the boundaries of CDR regions based on the location of structural loop regions (see, for example, Chothia&Lesk (1987) J. Mol. Biol. 196:901-917; Chothia et al. (1989) Nature 342:878-883).

The term “IMGT numbering system” herein generally refers to the immunoglobulin numbering system proposed by Chothia et al., which is a classical rule for identifying the boundaries of CDR regions based on the location of structural loop regions (see, for example, Chothia&Lesk (1987) J. Mol. Biol. 196: 901-917; Chothia et al. (1989) Nature 342: 878-883).

As used herein, the term “specific binding” refers to a binding reaction that determines the presence of an antigen in a heterogeneous population of proteins and other biomolecules, the proteins and other biomolecules are for example specifically recognized by an antibody or an antigen-binding fragment thereof. An antibody or an antigen-binding fragment thereof that specifically binds to an antigen would bind to the antigen with a KD of less than 100 nM. For example, an antibody or an antigen-binding fragment thereof that specifically binds to an antigen would bind to the antigen with a KD of up to 100 nM (for example, between 1 pM and 100 nM). An antibody or an antigen-binding fragment thereof that does not exhibit specific binding to a particular antigen or an epitope thereof would exhibit a KD for the particular antigen or the epitope thereof of greater than 100 nM (for example, greater than 500 nM, 1 μM, 100 μM, 500 μM, or 1 mM). Various immunoassays are available to select for an antibody that reacts specifically with a specific protein or carbohydrate. For example, solid-phase ELISA immunoassay is routinely used to select for an antibody that reacts specifically with a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), which describes immunoassay methods and conditions that can be used to determine specific immunoreactivity.

As used herein, the term “antibody conjugate” refers to a coupled entity/conjugate formed by chemically bonding an antibody molecule to another molecule either directly or through a linker. Examples include antibody-drug conjugate (ADC), in which the drug molecule is said another molecule.

The term “chimeric antigen receptor (CAR)” herein refers to a recombinant protein comprising at least (1) an extracellular antigen-binding domain, such as a variable heavy or light chain of an antibody, and (2) transmembrane domains used to make the anchored CAR enter immune effector cells, and (3) an intracellular signaling domain. In certain embodiments, the extracellular antigen binding domain of the CAR comprises a scFv. The scFv can be derived from the variable heavy and light regions of a fusion antibody. Alternatively or additionally, the scFv may be derived from Fab's (rather than an antibody, for example obtained from a Fab library). In certain embodiments, the scFv is fused to the transmembrane domain and then to the intracellular signaling domain.

Herein the term “nucleic acid” includes any compound and/or substance comprising a polymer of nucleotides. Each nucleotide consists of a base, specifically a purine or pyrimidine base (i.e., cytosine (C), guanine (G), adenine (A), thymine (T) or uracil (U)), a sugar (i.e., deoxyribose or ribose) and a phosphate group. Typically, the nucleic acid molecule is described by a sequence of bases, whereby the bases represent the primary structure (linear structure) of the nucleic acid molecule. The sequence of bases is usually expressed as 5′ to 3′. In this context, the term nucleic acid molecule encompasses deoxyribonucleic acid (DNA), including for example complementary DNA (cDNA) and genomic DNA, ribonucleic acid (RNA), especially messenger RNA (mRNA), synthetic forms of DNA or RNA, and a polymer containing a mixture of two or more of these molecules. The nucleic acid molecule can be linear or circular. Furthermore, the term nucleic acid molecule includes both sense strand and antisense strand, as well as single-stranded form and double-stranded form. Furthermore, the nucleic acid molecules described herein may contain naturally occurring or non-naturally occurring nucleotides. Examples of non-naturally occurring nucleotides include modified nucleotide bases with derivatized sugar or phosphate backbone linkages or chemically modified residues. The nucleic acid molecule also encompasses DNA and RNA molecules which are suitable as vectors for direct expression of the antibody of the invention in vitro and/or in vivo, for example in a host or patient. Such DNA (for example cDNA) or RNA (for example mRNA) vectors may be unmodified or modified. For example, mRNA can be chemically modified to enhance the stability of the RNA vector and/or the expression of the encoded molecule, so that the mRNA can be injected into a subject to generate an antibody in vivo (see, for example, Stadler et al., Nature Medicine 2017, Published online Jun. 12, 2017, doi: 10.1038/nm.4356 or EP 2101823 B1).

As used herein, the term “vector” includes a nucleic acid vector, such as a DNA vector (such as a plasmid), an RNA vector, a virus or other suitable replicons (such as viral vectors). A variety of vectors have been developed for the delivery of polynucleotides encoding foreign proteins into prokaryotic or eukaryotic cells. The expression vector of the invention contains polynucleotide sequences together with additional sequence elements, for example, for expressing proteins and/or integrating these polynucleotide sequences into the genome of mammalian cells. Certain vectors that can be used to express the antibody and the antibody fragment of the invention include plasmids that contain regulatory sequences (such as promoter and enhancer regions) that direct transcription of the gene. Other useful vectors for expressing the antibody and the antibody fragment contain polynucleotide sequences that enhance the rate of translation of these genes or improve the stability or nuclear export of mRNA transcripted from the genes. These sequence elements include, for example, 5′ and 3′ untranslated regions, internal ribosomal entry site (IRES), and polyadenylation signal site in order to direct the efficient transcription of the genes carried on the expression vector. The expression vector of the present invention may also contain a polynucleotide encoding a marker for selection of cells containing such a vector. Examples of suitable markers include genes encoding resistance to antibiotics such as ampicillin, chloramphenicol, kanamycin or nourseothyrcin.

The term “host cell” herein refers to a cell into which a foreign nucleic acid is introduced, including the progenys of such a cell. The host cell includes “transformant” and “transformed cell” which include the primary transformed cell and progeny derived therefrom, regardless of the number of passages. The progeny may not be identical to the parental cell in nucleic acid content, and may contain a mutation. The mutant progeny with the same function or biological activity as those screened or selected in the initially transformed cells are included herein.

As used herein, the term “pharmaceutical composition” refers to a preparation that is present in a form which allows the active ingredients contained therein to be biologically effective and does not contain additional ingredients that would be unacceptably toxic to the subject to which the pharmaceutical composition is administered.

As used herein, the terms “subject”, “object” and “patient” refer to an organism receiving treatment for a particular disease or condition, such as a cancer or an infectious disease, as described herein. Examples of subjects and patients include mammals, such as humans, primates, pigs, goats, rabbits, hamsters, cats, dogs, guinea pigs, members of the bovid family (cattle, bison, buffalo, elk, yak, etc.), sheep, and horses receiving treatment for a disease or a condition (for example, a cell proliferative disorder, such as a cancer or an infectious disease).

As used herein, the term “treatment” refers to surgical or therapeutic treatment, the purpose of which is to prevent, slow down (reduce) an undesired physiological change or pathology in the subject being treated, such as the progress of a cell proliferative disorder (such as a cancer or an infectious disease). Beneficial or desired clinical results include, but are not limited to, alleviation of symptoms, diminishment of extent of disease, stabilized (i.e., not worsening) state of disease, delay or slowing of disease progression, amelioration or palliation of the disease state and remission (whether partial response or complete response), whether detectable or undetectable. Subjects in need of treatment include subjects who already have a condition or a disease, subjects who are prone to a condition or a disease or subjects who intend to prevent a condition or a disease. When referring to the terms slow down, alleviation, diminishment, palliation, remission, etc., the meaning of eliminate, disappear, not occur, etc. is also included.

The term “effective amount” herein refers to an amount of a therapeutic agent effective to prevent or relieve a disease or a condition or the progression of the disease when administered alone or in combination with another therapeutic agent to a cell, tissue or subject. “Effective amount” also refers to an amount of a compound sufficient to relieve symptoms, for example, treat, cure, prevent or relieve the associated medical conditions, or to increase the rate of treatment, cure, prevent or relieve such conditions. When the active ingredient is administered to an individual alone, a therapeutically effective dose refers to the ingredient alone. When a combination is used, a therapeutically effective dose refers to the combined amounts of the active ingredients that produce a therapeutic effect, whether administered in combination, sequentially or simultaneously.

The term “appropriate condition” herein refers to a condition suitable for culturing various host cells, including eukaryotic cells and prokaryotic cells.

The term “cancer” herein refers to or describes the physiological condition in mammals that is typically characterized by unregulated cell growth. Both benign and malignant cancers are included in this definition.

The term “tumor” herein refers to all neoplastic cell growth and proliferation, whether malignant or benign, and to all pre-cancerous and cancerous cells and tissues. The terms “cancer” and “tumor” are not mutually exclusive when referred to herein.

The term “anti-tumor agent” herein refers to an anti-tumor drug, which is a class of drugs for the treatment of tumor diseases, including a chemotherapy drug, a biological agent and the like.

The term “EC50” herein refers to the half-maximal effective concentration, which includes the concentration of an antibody that induces a response halfway between baseline and maximum after a specified exposure time. EC50 essentially represents the concentration of an antibody at which 50% of its maximal effect is observed and which can be measured by methods known in the art.

The term “EC80” herein refers to the concentration of an antibody that elicits 80% of the maximal effect.

### BRIEF DESCRIPTION OF THE DRAWINGS

FIG. 1 shows the serum antibody titer of alpaca after immunization. FIG. 1A shows the serum antibody titer of alpaca immunized with human EGFR-ECD protein detected by ELISA; FIG. 1B shows the serum antibody titer of alpaca immunized with human EGFR-ECD protein detected by FACS.

FIG. 2 shows the detection results of EGFRvIII protein by SDS-PAGE reducing gel and non-reducing gel. M: marker; R (2 μg): loading quantity of sample 2 μg, reducing electrophoresis; NR (2 μg): loading quantity of sample 2 μg, non-reducing electrophoresis.

FIG. 3 shows the binding reaction of control antibody with human EGFRvIII protein detected by ELISA. Wherein the anti-EGFR vIII positive control antibody is: C225, 7D12 and 30D8, the negative control is hIgG1.

FIG. 4 shows the binding reaction of control antibody with human pepvIII protein detected by ELISA. The anti-pepvIII protein positive control antibody is 30D8, and the negative control is hIgG1.

FIG. 5 shows the FACS detection result of A431 cell; FIG. 5A shows the FACS result of detecting EGFR expression quantity in A431 cells by C225 antibody;

FIG. 5B shows the FACS result of detecting EGFRvIII expression quantity in A431 cells by 30D8 antibody. Wherein the negative control is hIgG1.

FIG. 6 shows the FACS result of MCF-7 cell; FIG. 6A shows the FACS result of detecting EGFR expression quantity in MCF-7 cells by C225 antibody; FIG. 6B shows the FACS result of detecting EGFRvIII expression quantity in MCF-7 cells by 30D8 antibody. Wherein the negative control is hIgG1.

FIG. 7 shows the FACS result of detecting CHO-K1 human EGFR by C225 antibody. Wherein the negative control is hIgG1.

FIG. 8 shows the FACS result of detecting HEK293T monkey EGFR by C225 antibody. Wherein the negative control is hIgG1.

FIG. 9 shows the FACS result of detecting CHO-K1 human EGFRvIII by 30D8 antibody. Wherein the negative control is hIgG1.

FIG. 10 shows the binding reaction of the VHH-Fc antibodies of the present invention with human EGFR protein detected by ELISA. Wherein the anti-EGFR positive control antibody is: C225, 7D12, negative control is hIgG1.

FIG. 11 shows the FACS detection result of the binding of the VHH-Fc antibodies of the present invention to the EGFR protein on the cell surface; FIG. 11A shows the binding reaction of the VHH-Fc of the present invention with human A431 cells detected by FACS; FIG. 11B shows the binding reaction of the VHH-Fc antibodies of the present invention with CHO-K1 human EGFR1D4 cells detected by FACS. Wherein the anti-EGFR positive control antibody is: C225, 7D12, negative control is hIgG1.

FIG. 12 shows the binding reaction of the VHH-Fc antibodies of the present invention with human EGFRvIII protein detected by ELISA. Wherein the anti-human EGFR vIII positive control antibody is: C225, 7D12 and 30D8, the negative control is hIgG1.

FIG. 13 shows the binding reaction of the VHH-Fc antibodies of the present invention with CHO-K1 human EGFRvIII1C6 cells detected by FACS. Wherein the anti-human EGFR vIII positive control antibody is: C225, 7D12 and 30D8, the negative control is hIgG1.

FIG. 14 shows the binding reaction of the VHH-Fc antibodies of the present invention with murine EGFR protein detected by ELISA. Wherein the negative control is hIgG1.

FIG. 15 shows the binding reaction of the VHH-Fc antibodies of the present invention with monkey EGFR protein detected by ELISA. Wherein the monkey anti-EGFR positive control antibody is: C225, 7D12, negative control is hIgG1.

FIG. 16 shows the binding reaction of the VHH-Fc antibodies of the present invention with HEK293T monkey EGFR cells detected by FACS. Wherein the anti-EGFR positive control antibody is: C225, 7D12, negative control is hIgG1.

FIG. 17 shows the specific binding reaction of the VHH-Fc antibodies of the present invention with the human EGFR protein on the cell surface detected by FACS. FIG. 17A shows the binding reaction of the VHH-Fc antibodies of the present invention with A431 cells detected by FACS; FIG. 17B shows the binding reaction of the VHH-Fc antibodies of the present invention with MCF-7 cells detected by FACS;

FIG. 17C shows the binding reaction of the VHH-Fc antibodies of the present invention with CHO-K1-human EGFR1D4 cells detected by FACS; FIG. 17D shows the binding reaction of the VHH-Fc antibodies of the present invention with CHO-K1 blank cells detected by FACS. Wherein the anti-EGFR positive control antibody is: C225, 7D12, negative control is hIgG1.

FIG. 18 shows the specific binding reaction of the VHH-Fc antibodies of the present invention with the monkey EGFR protein on the cell surface detected by FACS. FIG. 18A shows the binding reaction of the VHH-Fc antibodies of the present invention with HEK293T-monkey EGFR cells detected by FACS; FIG. 18B shows the binding reaction of the VHH-Fc antibodies of the present invention with 293 blank cells detected by FACS. Wherein the anti-EGFR positive control antibody is: C225, 7D12, negative control is hIgG1.

FIG. 19 shows the binding reaction of the VHH-Fc antibodies of the present invention with Her2 protein detected by ELISA.

FIG. 20 shows the binding of the VHH-Fc antibodies of the present invention with pepvIII detected by ELISA method. The anti-pepvIII positive control antibody is: 30D8, the negative control is hIgG1.

FIG. 21 shows the inhibition rate between VHH antibodies of the present invention detected by competitive ELISA.

FIG. 22 shows the antigen epitope classification of the VHH antibodies of the present invention.

### DETAILED DESCRIPTION OF EMBODIMENTS

The present invention is described in detail below in conjunction with examples and drawings, and the drawings herein is in order to illustrate some preferred embodiments of the present invention, however, it can be understood that the present invention is not limited to the disclosed specific embodiments or regarded as limiting the scope of the invention. If specific conditions are not specified in the examples, conventional conditions or conditions recommended by a manufacturer are followed. The reagents or instruments used therein for which manufacturers are not specified are all conventional products that are commercially available.

### Example 1: Screening of Nanobody Against Human EGFR

1.1 Immunization and Serum Titer Detection of Alpaca

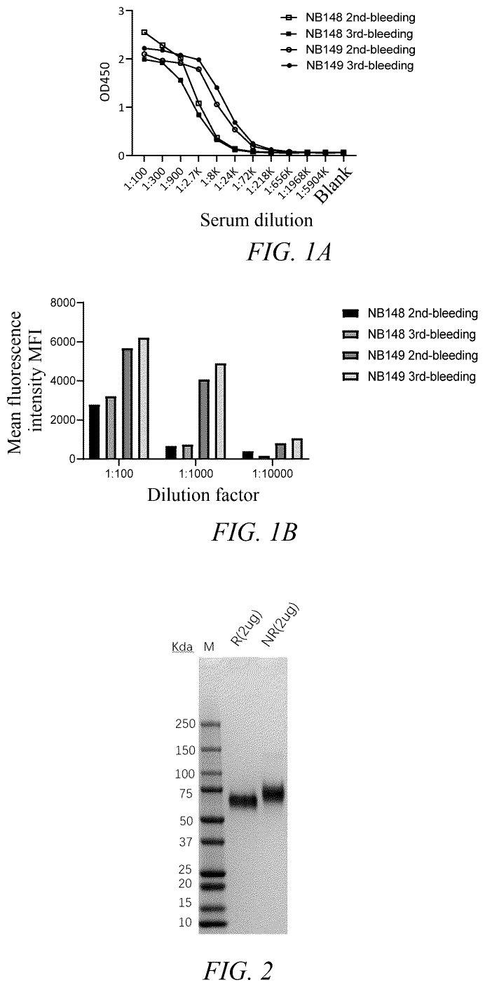

The human EGFR protein used for immunization was purchased from ACRO Biosystems (catalog number: EGR-H5222). Two alpacas (Llama) were selected for immunization, and each alpaca was immunized four times with an interval of 3 weeks. After the third immunization (TB2) and after the fourth immunization (TB3), peripheral blood was collected and serum was separated, and enzyme-linked immunosorbent assay (ELISA) and flow cytometry (FACS) were used to detect the antibody titer and specificity against human EGFR in serum, and the results are shown in FIG. 1A-1B and Table 1. Table 1 shows that the third immunization (TB2) serum titer has increased, and the fourth immunization (TB3) serum titer remains stable, alpaca can be used for VHH antibody library construction.

TABLE 1
The serum antibody titer of alpaca immunized with
human EGFR protein detected by ELISA

| Batch | OD450nM | | | |
|---|---|---|---|---|
| Dilution | NB148 | NB148 | NB149 | NB149 |
| factor | (TB2) | (TB3) | (TB2) | (TB3) |
| 1:100 | 2.56 | 1.99 | 2.10 | 2.22 |
| 1:300 | 2.28 | 1.92 | 1.97 | 2.18 |
| 1:900 | 2.02 | 1.56 | 1.91 | 2.08 |
| 1:2700 | 1.08 | 0.84 | 1.79 | 1.99 |
| 1:8100 | 0.37 | 0.32 | 1.06 | 1.41 |
| 1:24300 | 0.13 | 0.12 | 0.54 | 0.68 |
| 1:72900 | 0.08 | 0.07 | 0.19 | 0.25 |
| 1:218700 | 0.07 | 0.06 | 0.11 | 0.12 |
| 1:656100 | 0.06 | 0.05 | 0.08 | 0.08 |
| 1:1968300 | 0.07 | 0.06 | 0.06 | 0.07 |
| 1:5904900 | 0.06 | 0.05 | 0.06 | 0.06 |
| Blank | 0.07 | 0.06 | 0.05 | 0.06 |

1.2 Library Construction

A total of 100 mL of alpaca peripheral blood was collected after three immunizations and after four immunizations. Lymphocyte separation medium was used to isolate PBMC, RNAiso Plus reagent (Takara, catalog number: #9108/9109) was used to extract total RNA, and PrimeScript™ II 1st Strand cDNA Synthesis Kit (Takara, catalog number: 6210A) was used to reverse transcribe the extracted RNA into cDNA. Nested PCR was used to amplify the variable region nucleic acid fragment encoding the heavy chain antibody:
the first round of PCR:

(SEQ ID NO. 1)
upstream primer: CTTGGTGGTCCTGGCTGC
(SEQ ID NO. 2)
downstream primer: GOTACGTGCTGTTGAACTGITCC

the second round of PCR:
the first round PCR product was used as a template,

upstream primer:
(SEQ ID NO. 3)
CATGCCATGACTGTGGCCCAGGCGGCCCAGKTGCAGCTCGTGGAGTC
downstream primer-1:
(SEQ ID NO. 43
CATGCCATGACTCGCGGCCGGCCTGGCCATGGGGGTCTTOGCTGTGG
TGCG
downstream primer-2:
(SEQ ID NO. 5)
CATGCCATGACTCGCGGCCGGCCTGGCCGTCTTGTGGTTTTGGTGTC
TTGGG

The nucleic acid fragment of the nanobody of interest was recovered and cloned into the phage display vector pcomb3XSS (from Sichuan NB Biolab Co., Ltd) using the restriction endonuclease SfiI (NEB, catalog number: R0123S). The product was then electrotransformed into Escherichia coli electroporation competent cell TG1, and a nanobody phage display library target against EGFR was constructed and tested. By serial dilution plating, the calculated capacity is 2.0×10⁹. To test the insertion rate of the library, 48 clones were randomly selected for colony PCR, and the results showed that the insertion rate reached 100%.

1.3 Panning of Nanobody Against EGFR

The plate was coated with human EGFR-His-tagged fusion protein (ACRO Biosystems, catalog number: EGR-H5222) at 0.5 μg/well, and placed at 4° C. overnight; The next day, after blocking with 3% BSA-PBS at 37° C. for 1 hour, 100 μl of phage display library was added and incubated at 37° C. for 1 hour; After that, the plate was washed 6 times with PBST and 2 times with PBS to wash away unbound phages. Finally, 100 μL of Gly-HCl eluent was added to elute the phages that specifically bind to EGFR to enrich positive clones.

1.4 Screening of Specific Single Positive Clones by Phage Enzyme-Linked Immunoassay

After panning, blank Escherichia coli were infected with the obtained human EGFR-binding positive phages and plated. Then 96 single colonies were selected for proliferation and culture. The plates were coated with human EGFR-His protein respectively at 4° C. overnight, the phage culture supernatant was added, and incubated at 37° C. for 1 hour. 1:1000 diluted M13 antibody anti-M13-HRP labeled with horseradish peroxidase was added (NBbiolab, catalog number: 5004H). After washing, TMB chromogenic solution was added to develop color, and the optical density was measured at a wavelength of 450 nm. Human EGFR-positive clones were selected for sequencing. The sequencing results were analyzed using MOE software, and the evolutionary tree was constructed according to the amino acid sequence coding the VHH protein. According to the sequence similarity, 22 clones were obtained by eliminating the sequences that were close to each other on the evolutionary tree, and the CDR sequences of the 22 clones were analyzed by KABAT, Chothia or IMGT software respectively, and the corresponding sequence information is shown in the following table 2-4. Wherein Table 2 shows the antibody sequences represented by amino acids of 22 nanobody molecules, and Table 3 shows the antibody sequences represented by nucleotides of 22 nanobody molecules, and Table 4 shows the results of IMGT, Kabat and Chothia analysis of the CDRs of 22 nanobody molecules. Subsequently, the production and identification of VHH nanobody Fc fusion protein were carried out.

TABLE 2
Specific amino acid sequence information of the heavy chain variable
region of the anti-EGFR antibody

| Antibody | Sequence | |
|---|---|---|
| number | number | Heavy chain variable region sequence (VH) |
| S00N-NB1 | SEQ ID NO. 19 | QVQLVESGGGLVQAGGSLRLSCAAFISTFGRTDMTWYRQRSGNERE |
| 48-10 | | FVARISSGGSTIYADSAKGRFTISRDNVKNTVYLQMNSLTPEDTAVYYC |
| | | NTVPPRGSWSQGTQVTVSS |
| S00S-NB1 | SEQ ID NO. 21 | EVQWVESGGGLVQPGGSLRLSCAASDDISSIYTMAWYRQAPGKQREL |
| 48-13 | | VALVTPGGGTNYADSVKGRFTISRDNAKNTVYLQMNSLKPEDTAVYY |
| | | GNARHRVTGFAYWGQGTQVTVSS |
| S008-NB1 | SEQ ID NO. 23 | EVQVVESGGGLVQAGGSLRLSCAASGSTFSSYAMTWYRQAPGKQRE |
| 48-2 | | LVAAISSGGSTNYAASVKGRFTISRDNAKNTLYLQMNTLKPEDTAVYY |
| | | CNTDWGNGFSAEYDYWGQGTQVTVSS |
| S008 NB1 | SEQ ID NO. 25 | EVQLVESGGGLVQPGGSLRLSCAVSQSISSINAMDWYRQAPGKQREL |
| 48-25 | | VARENNGRTNYADSVKGRFTISRDNARNTVYLQMNSLKPEDTAVYYC |
| | | NIVPPLRNWGQGTQVTVSS |
| S008-NB1 | SEQ ID NO. 27 | EVQLVESGGGLVRAGGSLRLSCAASGTISSYDVVGWYRQAPGKQRE |
| 48-30 | | LVALIGTDRWLNLGDFAKGRFTMSTDDAANTVDLEMNSLKPEDTAVY |
| | | YCYTFQHTVGPRWGQGTQVTVSS |
| S008-NB1 | SEQ ID NO. 29 | QLQLVESGGGLVQAGGSLKLSCVASGFTFSNYAMAWYRQAPGKQRE |
| 48-48 | | LIASCSASCIWINYGASVKGRFTMSLDNAKKTVYLQMDSLKPEDTAVY |
| | | YCRNLDANYWGQGTQVTVSS |
| S008-NB1 | SEQ ID NO. 31 | QLQLVESGGGLVQAGGSLRLSCAVSISTFTINGWDWYRQAPGKQRE |
| 48-5 | | LVALISNDGTTTYTDSVKGRFTISRDGAKNTVYLQMNNLKPEDTAVYY |
| | | CNTIPPAGSWGQGTQVTVSS |
| S008-NB1 | SEQ ID NO. 33 | EVQLVESGGGLVQAGGSLRLSCAASGFRIINDRMAWYRQAPGKQRE |
| 48-52 | | AVASIDYAGSTTYAEFVKGRFTISRDNTKNMVTLQMNNLEPEDTAVYY |
| | | CNTAPIARFRGQGTQVTVSS |
| S008-NB1 | SEQ ID NO. 35 | EVQVVESGGGLVQAGGSLRLSCAASTITISSAGVSWYRQAPGKQREL |
| 48-69 | | VAIITRGGSTNYADSVKGRFTISRDNAKNTHYLQMYNLKPEDTGIYYCN |
| | | WPPTYWGQGTQVTVSS |
| S008-NB1 | SEQ ID NO. 37 | QVQLVESGGGLVQAGGSLRLSCAASEGTLSSEAMGWHRLAPGKQRE |
| 48-70 | | SVGFISSGGSTNYRDSVKGRFTISRDNAKNTVYLQMNSLKPEDIGVYF |
| | | CRIPNSVGPWGQGTQVTVSS |
| S008-NB1 | SEQ ID NO. 39 | EVQVVESGGGLVQAGGSLRLSCVASGFDFSDSVMGWYRQAPGKQR |
| | | EAVAIISSVGQSNYRDSVQGRFTVSRSNTENTMYLQMDSLKPEDTAIY |
| | | YCKKFGPGEYWGQGTQVTVSS |
| S008-NB1 | SEQ ID NO. 41 | EVQLVESGGGLVQAGGSLRLSCVVSGSTFSSYAMGWYRQAPGKQR |
| | | ELVAAISSGVSANVADSLKGRFAISRDNAKNAVYLQMNSLKPEDTAVY |
| | | YCNTLPRSMPYWGKGTLVTVSS |
| S008-NB1 | SEQ ID NO. 43 | EVQLVESGGGLVQAGGSLRLSCAASGSTLSSYAMGWYRQAPGKQRE |
| 48-9 | | LVAAISSGGGSTNYRDSVKGRFTISRDNAKNTVYLQMNSLKPEDTAVY |
| | | YCKSTDYGSLFDSWGQGTQVTVSS |
| S008-NB1 | SEQ ID NO. 45 | EVQVVESGGGTVQAGGSLRLSCAASARTFANAHMAWFRQAPGKERE |
| 49-1 | | FLAAITWSGGITDYANSVKGRFTISRDNAENAMYLQMNSLKPEDTAIYI |
| | | CAVDTTSAYDQLWSRQSEYEYWGQGTQVTVSS |
| S008-NB1 | SEQ ID NO. 47 | QVQLVESGGGLVQPGGSLRLSCVVSESISVINAMTWYRQAPGKQREL |
| 49-12 | | VALISRGGSTNYADSVKGRFTISRDNAKNSVYLQMNSLKPEDIALYYC |
| | | NVVPPLGSWGQGTQVTVSS |
| S008-NB1 | SEQ ID NO. 49 | QLQLVESGGGLVRPGGSLRLSCAASGSIFSRSGATWYRQAPGKQRE |
| 49-45 | | LVALLTRDGHTDYPVVSVKGRFTISKDNAKNTVYLQMNSLQPEDTAVY |
| | | YCNAIPPLGSWGRGTQVTVSS |
| S008-NB1 | SEQ ID NO. 51 | QVQLVESGGGLVQPGGSLRLSCAASGFSLDAYAIGWFRQAPGMGRE |
| 49-2 | | GVSCISSAGNTDYADSVKGRFAISRDNAKNTVYLQMNSLKPEDSGVY |
| | | YCARTSEGVYRGRLACALYESAADFRSGGQGTRVTVSS |
| S008-NB1 | SEQ ID NO. 53 | EVQVVESGGGLVQPGGSLRLSCAASGSGFTINAMTWYRRAPGKERE |
| 49-20 | | LVAIITNGGITNYADSVKGRFTISRDNAKSTVYLQMDGLEPEDTAVYYC |
| | | NIVPPVYWGQGTQVTVSS |
| S008-NB1 | SEQ ID NO. 55 | QVQLVESGGGLVQAGGSLRLSCVASGRPPSIYRMAWFRQAPGKERD |
| 49-22 | | FVAAINWGGTATYYEDSVKGRFTISRDNTKNTVWLQMNSLKPEDTAV |
| | | YYCAAGTGTTYTPQRGDAYGYWGQGTQVTVSS |
| S008-NB1 | SEQ ID NO. 57 | QLQLVESGGGLVQPGGSLRLSCAASRSISSINTMTWYRYQGPGKERE |
| 49-60 | | LVALITLGGTTNYADSVKGRFTISRDDAKNTLYLEMNSLKPEDTAVYYC |
| | | NAVPPFRWGQGTQVTVSS |
| S008-NB1 | SEQ ID NO. 59 | EVQVVESGGGLVQPGGSLRLSCVASGIIFSSYAMGWYRQAPGKQREL |
| 49-64 | | VARISSGGGLYYEDPVKGRFTISRDNARNTVYLQMSSVKPEDTAVYYC |
| | | NVVPYTPGYWGQGTQVTVSS |
| S008-NB1 | SEQ ID NO. 61 | QVQLVESGGFVQAGGSLRLSCAASGRTFSKYAMGWFRQAPGKEREF |
| 49-9 | | VAAIRWIGGSTYYADSVKGRFTISRDNDKNTLYLQMNSLKPEDTAVYY |
| | | CAAGYQAYPEPPWEYDYWGQGTQVTVSS |

TABLE 3
Specific nucleotide sequence information of the heavy chain
variable region of the anti-EGFR antibody

| Antibody | Sequence | |
|---|---|---|
| number | number | Heavy chain variable region sequence (VH) |
| S008-NB1 | SEQ ID NO. 20 | CAGGTGCAGCTGGTGGAGAGCGGGGGGGGCCTGGTGCAGGCCG |
| 48.10 | | GQGGCAGCCTGAGGCTGAGCTGQGCCGCCTTCATCAGCACCTTCG |
| | | GCAGGACCGACATGACCTGGTACAGGCAGAGGAGCGGCAACGAG |
| | | AGGGAGTTCGTGGCCAGGATCAGCAGCGGCGGCAGCACCATCTA |
| | | CGCCGACAGCGCCAAGGGCAGGTTCACCATCAGCAGGGACAACG |
| | | TGAAGAACACCGTGTACCTGCAGATGAACAGCCTGACCCCCGAGG |
| | | ACACCGCCGTGTACTACTGCAACACCGTGCCCCCCAGGGGCAGCT |
| | | GGAGCCAGGGCACCCAGGTGACCGTGAGCAGC |
| S008-NB1 | SEQ ID NO. 22 | GAGGTGCAGGTGGTGGAGAGCGGCGGCGGCCTGGTGCAGCCCG |
| 48-13 | | GCGGCAGCCTGAGGCTGAGCTGQGCCCCAGCGACGACATCAGC |
| | | AGCATCTACACCATGGCCTGGTACAGGCAGGCCCCCGGCAAGCAG |
| | | AGGGAGCTGGTGGCCCTGGTGACCCCCGGCGGGGGCACCAACTA |
| | | CGCCGACAGCGTGAAGGGCAGGTTCACCATCAGCAGGGACAACG |
| | | CCAAGAACACCGTGTACCTGCAGATGAACAGCCTGAAGCCCGAGG |
| | | ACACCGCCGTGTACTACTGCAACGCCAGGCACAGGGTGACCGGCT |
| | | TCGCCTACTGGGGCCAGGGCACCCAGGTGACCGTGAGCAGC |
| S008-NB1 | SEQ ID NO. 24 | GAGGTGCAGGTGGTGGAGAGCGGGGGGGGCCTGGTGCAGGCCG |
| 48-2 | | GCGGCAGCCTGAGGCTGAGCTGCGCCGCCAGCGGCAGCACCTTC |
| | | AGCAGCTACGCCATGACCTGGTACAGGCAGGCCCCCGGCAAGCA |
| | | GAGGGAGGTGGTGGCCGCCATCAGCAGCGGCGGCAGCACCAACT |
| | | ACGCCGCCAGCGTGAAGGGCAGGTTCACCATCAGCAGGGACAAC |
| | | GCCAAGAACACCCTGTACCTGCAGATGAACACCCTGAAGCCCGAG |
| | | GACACCGCCGTGTACTACTGCAACACCGAGTGGGGCAACGGCTTC |
| | | AGCGCCGAGTACGACTACTGGGGCCAGGGCACCCAGGTGACCGT |
| | | GAGCAGG |
| S008-NB1 | SEQ ID NO. 26 | GAGGTGCAGCTGGTGGAGAGGGGGGGGGGCCTGGTGCAGQGCCCG |
| 48-25 | | GCGGCAGCCTGAGGCTGAGCTGCGCCGTGAGCGAGAGCATCAGC |
| | | AGCATCAACGCCATGGACTGGTACAGGCAGGCCCCCGGCAAGCA |
| | | GAGGGAGCTGGTGGCCATCATCTICAACAACGGCAGGACCAACTA |
| | | CGCCGACAGCGTGAAGGGCAGGTTCACCATCAGCAGGGACAACG |
| | | CCAGGAACACCGTGTACCTGCAGATGAACAGCCTGAAGCCCGAGG |
| | | ACACCGCCGTGTACTACTGCAACATCGTGCCCCCCCTGAGGAACT |
| | | GGGGCCAGGGCACCCAGGTGACCGTGAGCAGC |
| S008-NB1 | SEQ ID NO. 28 | GAGGTGCAGCTGGIGGAGAGCGGGGGGGGCCTGGTGAGGGCCG |
| 48-3 | | GCGGCAGCCTGAGGCTGAGCTGCGCCGCCAGCGGCACCATCAGC |
| | | AGCTACGACGTGGTGGGCTGGTACAGGCAGGCCCCCGGCAAGCA |
| | | GAGGGAGCTGGTGGCCCTGATCGGCACCGACAGGTGGCTGAACC |
| | | TGGGCGACTTCGCCAAGGGCAGGTTCACCATGAGCACCGACGAC |
| | | GCCGCCAACACCGTGGACCTGGAGATGAACAGCCTGAAGCCCGA |
| | | GGACACCGCCGTGTACTACTGCTACACCTTCCAGCACACCGTGGG |
| | | CCCCAGGTGGGGCCAGGGCACCCAGGTGACCGTGAGCAGC |
| S008-NB1 | SEQ ID NO. 30 | CAGCTGCAGCTGGTGGAGAGCGGGGGGGGCCTGGTGCAGGCCG |
| 48-48 | | GCGGCAGCCTGAAGCTGAGCTGCGTGGCCAGCGGCTTCACCTTCA |
| | | GCAACTACGCCATGGCCTGGTACAGGCAGGCCCCCGGCAAGCAG |
| | | AGGGAGCTGATCGCCAGCTGCAGQGCCAGCTGCATCTGGACCAAC |
| | | TACGGCGCCAGCGIGAAGGGGAGGTTCACCATGAGCCTGGACAAC |
| | | GCCAAGAAGACCGTGTACCTGCAGATGGACAGCCTGAAGCCCGAG |
| | | GACACCGCCGTGTACTACTGCAGGAACCTGGACGCCAACTACTGG |
| | | GGCCAGGGCACCCAGGTGACCGTGAGCAGC |
| S008-NB1 | SEQ ID NO. 32 | CAGCTGCAGCTGGTGGAGAGCGGGGGGGGCCTGGTGCAGGCCG |
| 48-5 | | GCGGCAGCCTGAGGCTGAGCTGCGCCGTGAGCATCAGCACCTTC |
| | | ACCACCAACGGCTGGGACTGGTACAGGCAGGCCCCCGGCAAGCA |
| | | GAGGGAGCTGGTGGCCCTGATCAGCAACGACGGCACCACCACCT |
| | | ACACCGACAGCGTGAAGGGCAGGTTCACCATCAGCAGGGACGGC |
| | | GCCAAGAACACCGTGTACCTGCAGATGAACAACCTGAAGCCCGAG |
| | | GACACCGCCGTGTACTACTGCAACACCATCCCCCCCGCCGGCAGG |
| | | TGGGGCCAGGGCACCCAGGTGACCGTGAGCAGC |
| S008-NB1 | SEQ ID NO. 34 | GAGGTGCAGCTGGTGGAGAGCGGGGGGGGCCTGGTGCAGGCCG |
| 48-52 | | GCGGCAGCCTGAGGCTGAGCTGCGCCGCCAGQGGCTTCAGGATC |
| | | ATCAACGACAGGATGGCCTGGTACAGGCAGGCCCCCGGCAAGCA |
| | | GAGGGAGGCCGTGGCCAGCATCGACTACGCCGGCAGCACCACCT |
| | | ACGCCGAGTTCGTGAAGGGCAGGTTCACCATCAGCAGGGACAACA |
| | | CCAAGAACATGGTGACCCTGCAGATGAAGAACCTGGAGCCCGAGG |
| | | ACACCGCCGTGTACTACTGCAACACCGCCCCCATCGCCAGGTTCA |
| | | GGGGCCAGGGCACCCAGGTGACCGTGAGCAGC |
| S008-NB1 | SEQ ID NO. 36 | GAGGTGCAGGTGGTGGAGAGCGGCGGCGGCCTGGTGCAGGCCG |
| 48-69 | | GCGGCAGCCTGAGGCTGAGCTGCGCCGCCAGCACCATCACCATC |
| | | AGCAGQGCCGGCGTGAGCIGGTACAGGCAGGCCCCCGGCAAGCA |
| | | GAGGGAGCTGGTGGCCATCATCACCAGGGGGGGCAGCACCAACT |
| | | ACGCCGACAGCGTGAAGGGCAGGTTCACCATCAGCAGGGACAAC |
| | | GCCAAGAACACCCACTACCTGCAGATGTACAACCTGAAGCCCGAG |
| | | GACACCGGCATCTACTACTGCAACGTGGTGCCCCCCACCTACTGG |
| | | GGCCAGGGGACCCAGGTGACCGTGAGCAGG |
| S008-NB1 | SEQ ID NO. 38 | CAGGTGCAGGTGGTGGAGAGCGGGGGGGGCCTGGTGCAGGCCG |
| 48-70 | | GGGGCAGCCTGAGGCTGAGCTGCGCCGCCAGCGAGGGCACCCTG |
| | | AGCAGCGAGGCCATGGGCTGGCACAGGCIGGCCCCCGGCAAGCA |
| | | GAGGGAGAGCGTGGGCTTCATCAGCAGCGGCGGCAGCACCAACT |
| | | ACAGGGACAGCGTGAAGGGCAGGTTCACCATCAGCAGGGACAAC |
| | | GCCAAGAACACCGTGTACCTGCAGATGAACAGCCTGAAGCCCGAG |
| | | GAGACCGGCGTGTACTICTGCAGGATCCCCAACAGQGTGGGCCCC |
| | | TGGGGCCAGGGCACCCAGGTGACCGTGAGCAGC |
| S008-NB1 | SEQ ID NO. 40 | GAGGTGCAGGTGGTGGAGAGCGGCGGGGGCCTGGTGCAGGCCG |
| 48-77 | | GCGGCAGCCTGAGGCTGAGCTGCGTGGCCAGCGGCTTCGACTTC |
| | | AGCGACAGCGTGATGGGCTGGTACAGGCAGGCCCCCGGCAAGCA |
| | | GAGGGAGGCCGTGGCCATCATCAGCAGCGTGGGCCAGAGCAACT |
| | | ACAGGGACAGCGTGCAGGGCAGGTTCACCGTGAGCAGGAGCAAC |
| | | ACCGAGAACACCATGTACCTGCAGATGGACAGCCTGAAGCCCGAG |
| | | GACACCGCCATCTACTACTGCAAGAAGTTCGGCCCCGGCGAGTAC |
| | | TGGGGCCAGGGCACCCAGGTGACCGTGAGCAGC |
| S008-NB1 | SEQ ID NO. 42 | GAGGTGCAGCTGGTGGAGAGCGGCGGCGGCCTGGTGCAGGCCG |
| 48-8 | | GCGGCAGCCTGAGGCTGAGCTGCGTGGTGAGCGGCAGCACCTTC |
| | | AGCAGCTACGCCATGGGCTGGTACAGGCAGGCCCCCGGCAAGCA |
| | | GAGGGAGCTGGTGGCCGCCATCAGCAGGGGCGTAGCGCCAACG |
| | | TGGCCGACAGCCTGAAGGGCAGGTTCGCCATCAGCAGGGACAAC |
| | | GCCAAGAACGCCGTGTACCTGCAGATGAACAGCCTGAAGCCCGAG |
| | | GACACCGCCGTGTACTACTGCAACACCCTGCCCAGGAGCATGCCC |
| | | TACTGGGGCAAGGGCACCCTGGTGACCGTGAGCAGC |
| S008-NB1 | SEQ ID NO. 44 | GAGGTGCAGCTGGTGGAGAGGGGGGGGCCTGGTGCAGGCCG |
| 48-9 | | GCGGCAGCCTGAGGCTGAGCTGCGCCGCCAGCGGCAGCACCCTG |
| | | AGCAGCTACGCCATGGGCTGGTACAGGCAGGCCCCCGGCAAGCA |
| | | GAGGGAGCTGGTGGCCGCCATCAGCAGCGGGGGGGGCAGCACCA |
| | | ACTACAGGGACAGCGTGAAGGGCAGGTTCACCATCAGCAGGGACA |
| | | ACGCCAAGAACACCGTGTACCTGCAGATGAACAGCCTGAAGCCCG |
| | | AGGACACCGCCGTGTACTACTGCAAGAGCACCGACTACGGCAGCC |
| | | TGTTCGACAGCTGGGGCCAGGGCACCCAGGTGACCGTGAGCAGC |
| S008-NB1 | SEQ ID NO. 46 | GAGGTGCAGGTGGTGGAGAGCGGCGGCGGCACCGTGCAGGCCG |
| 49.1 | | GCGGCAGCCTGAGGCTGAGCTGCGCCGCCAGCGCCAGGACCTTC |
| | | GCCAACGCCCACATGGCCTGGTTCAGGGAGGCCCCCGGCAAGGA |
| | | GAGGGAGTTCCTGGGGGGCATCACGTGGAGGGGCGGCATCACGG |
| | | ACTACGCCAACAGCGTGAAGGGCAGGTTCACCATCAGCAGGGACA |
| | | ACGCCGAGAACGCCATGTACCTGCAGATGAACAGCOTGAAGCCCG |
| | | AGGACACCGCCATCTACATCTGCGCCGTGGACACCACCAGCGCCT |
| | | ACGACCAGCTGTGGAGCAGGCAGAGCGAGTACGAGTACTGGGGC |
| | | CAGGGGACCCAGGTGACCGTGAGCAGC |
| S008-NB1 | SEQ ID NO. 48 | CAGGTGCAGCTGGTGGAGAGCGGGGGGGGCCTGGTGCAGOCCG |
| 49-12 | | GCGGCAGCCTGAGGCTGAGCTGCGTGGTGAGCGAGAGCATCAGO |
| | | GTGATCAACGCCATGACCTGGTACAGGCAGGCCCCCGGCAAGCA |
| | | GAGGGAGCTGGTGGCCCTGATCAGCAGGGGGGGCAGCACCAACT |
| | | ACGCCGACAGCGTGAAGGGCAGGTTCACCATCAGCAGGGACAAC |
| | | GCCAAGAACAGCGTGTACCTGCAGATGAACAGCCTGAAGCCCGAG |
| | | GACACCGCCCTGTACTACTGCAACGTGGTGCCCCCCCTGGGCAGC |
| | | TGGGGCCAGGGCACCCAGGTGACCGTGAGCAGC |
| S008-NB1 | SEQ ID NO. 50 | CAGCTGCAGCTGGTGGAGAGCGGGGGGGGCCTGGTGAGGCCCG |
| 49.15 | | GCGGCAGCCTGAGGCTGAGCTGCGCCGCCAGCGGCAGCATCTTC |
| | | AGCAGGAGCGGCGCCACCTGGTACAGGCAGGCCCCCGGCAAGCA |
| | | GAGGGAGCTGGTGGCCCTGCTGACCAGGGACGGCCACACCGACT |
| | | ACCCCGTGGTGAGCGTGAAGGGCAGGTTCACCATCAGCAAGGACA |
| | | ACGCCAAGAACACCGTGTACCTGCAGATGAACAGOCTGCAGCCCG |
| | | AGGACACCGCCGTGTACTACTGCAACGCCATCCCCCCCCTGGGCA |
| | | GCTGGGGCAGGGGCACCCAGGTGACCGTGAGCAGC |
| S008-NB1 | SEQ ID NO. 52 | CAGGTGCAGOTGGTGGAGAGCGGGGGGGGCCTGGTGCAGCCCG |
| 49.7 | | GCGGCAGCCTGAGGCTGAGCTGCGCCGCCAGCGGCTTCAGCCTG |
| | | GACGCCTACGCCATCGGCTGGTTGAGGCAGGCCCCCGGCATGGG |
| | | CAGGGAGGGCGTGAGCTGCATCAGCAGCGCCGGCAACACCGACT |
| | | ACGCCGACAGCGTGAAGGGGAGGTTCGCCATCAGCAGGGACAAC |
| | | GCCAAGAACACCGTGTACCTGCAGATGAACAGCCTGAAGCCCGAG |
| | | GACAGCGGCGTGTACTACTGCGCCAGGACCAGCGAGGGCGTGTA |
| | | CAGGGGCAGGCTGGCCTGCGCCCTGTACGAGAGCGCCGCCGACT |
| | | TCAGGAGCGGCGGCCAGGGCACCAGGGTGACCGTGAGCAGC |
| S008-NB1 | SEQ ID NO. 54 | GAGGTGCAGGTGGTGGAGAGCGGCGGCGGCCTGGTGCAGCCCG |
| 49-25 | | GCGGCAGCCTGAGGCTGAGCTGCGCCGGCAGCGGCAGCGGCTTC |
| | | ACCATCAACGCCATGACGTGGTACAGGAGGGCCCCCGGCAAGGA |
| | | GAGGGAGCTGGTGGCCATCATCACCAACGGCGGCATCACCAACTA |
| | | CCCCGACAGCGTGAAGGGCAGGTTCACCATCAGCAGGGACAACG |
| | | CCAAGAGCACCGTGTACCTGCAGATGGACGGCCTGGAGCCCGAG |
| | | GACACCGCCGTGTACTACTGCAACATCGTGCCCCCCGTGTACTGG |
| | | GGCCAGGGCACCCAGGTGACCGTGAGCAGC |
| S008-NB1 | SEQ ID NO. 56 | CAGGTGCAGCTGGTGGAGAGCGGCGGCGGCCTGGTGCAGGGGG |
| 49-22 | | GCGGCAGCCTGAGGCTGAGCTGCGTGGCCAGCGGCAGGTTCCCC |
| | | AGCATCTACAGGATGGCCTGGTTCAGGCAGGCCCCCGGCAAGGA |
| | | GAGGGACTTCGTGGCCGCCATCAACTGGGGCGGCACCGCCACCT |
| | | ACTACGAGGACAGCGTGAAGGGCAGGTTCACCATCAGCAGGGACA |
| | | ACACCAAGAACACCGTGTGGCTGCAGATGAACAGCCTGAAGCCCG |
| | | AGGACACCGCCGTGTACTACTGQGCCGCOGGCACGGGCACCACC |
| | | TACACCCCCCAGAGGGGCGACGCCTACGGCTACTGGGGCCAGGG |
| | | CACCCAGGTGACCGTGAGCAGC |
| S008-NB1 | SEQ ID NO. 58 | CAGCTGCAGCTGGTGGAGAGCGGCGGGGGCCTGGTGCAGCCCG |
| 19-60 | | GCGGCAGCCTGAGGCTGAGCTGCGCCGCCAGCAGGAGCATCAGC |
| | | AGCATCAACACCATGACCTGGTACAGGTACCAGGGCCCCGGCAAG |
| | | GAGAGGGAGCTGGTGGCCCTGATCACCCTGGGCGGCACCACCAA |
| | | CTACGCCGACAGCGTGAAGGGCAGGTTCACCATCAGCAGGGACG |
| | | ACGCCAAGAACACCCTGTACCTGGAGATGAACAGCCTGAAGCCCG |
| | | AGGACACCGCCGTGTACTACTGCAACGCCGTGCCCCCCTTCAGGT |
| | | GGGGCCAGGGCACCCAGGTGACCGTGAGCAGC |
| S008-NB1 | SEQ ID NO. 60 | GAGGTGCAGGTGGTGGAGAGCGGGGGGGGCCTGGTGCAGCCCG |
| 49-64 | | GCGGCAGCCTGAGGCTGAGCTGCGTGGCCAGCGGCATCATCTTC |
| | | AGCAGCTACGCCATGGGCTGGTADAGGCAGGCCCCCGGCAAGCA |
| | | GAGGGAGCTGGTGGCCAGGATCAGCAGCGGGGGCGGCCTGTACT |
| | | ACGAGGACCCCGTGAAGGGCAGGTTCACCATCAGCAGGGACAAC |
| | | GCCAGGAACACCGTGTACCTGCAGATGAGGAGCGTGAAGCCCGA |
| | | GGACACCGCCGTGTACTACTGCAACGTGGTGCCCTACACCCCCGG |
| | | CTACTGGGGCCAGGGCACCCAGGTGACCGTGAGCAGC |
| S008-NB1 | SEQ ID NO. 62 | CAGGTGCAGCTGGTGGAGAGCGGCGGCTTCGTGCAGGCCGGGGG |
| 49-9 | | CAGCCTGAGGCTGAGCTGCGCOGCCAGCGGCAGGACCTTCAGCA |
| | | AGTACGCCATGGGCTGGTTCAGGCAGGCCCCCGGCAAGGAGAGG |
| | | GAGTTCGTGGCCGCCATCAGGTGGATCGGCGGCAGCACCTACTAC |
| | | GCCGACAGCGTGAAGGGCAGGTTCACCATCAGCAGGGACAACGA |
| | | CAAGAACACCCTGTACCTGCAGATGAACAGCCTGAAGCCCGAGGA |
| | | CACCGCCGTGTACTACTGCGCCGGGGGCTACCAGGCCTACCCCGA |
| | | GCCCCCCTGGGAGTACGACTACTGGGGCCAGGGCACCCAGGTGA |
| | | CCGTGAGCAGC |

TABLE 4
Specific sequence information of CDRs of EGFR nanobody analyzed by
IMGT, KABAT and Chothia software

| | | | | | | |
|---|---|---|---|---|---|---|
| Antibody | Sequence | | | | | |
| number | number | CDR1 | Sequence | COR2 | Sequence | CDR3 |

IMGT analysis

| | | | | | | |
|---|---|---|---|---|---|---|
| S008-NB1 | SEQ ID | ISTFGRTD | SEQ ID | ISSGGST | SEQ ID | NTVPPRGS |
| 48-10 | NO. 63: | | NO. 64 | | NO. 65 | |
| S008-NB1 | SEQ ID | DDISSIYT | SEQ ID | VTPGGGT | SEQ ID | NARHRVTGF |
| 48-13 | NO. 72 | | NO. 73 | | NO. 74 | AY |
| S008-NB1 | SBO ID | GSTESSYA | SEQ ID | ISSGGST | SEQ ID | NTDWGNGFS |
| 48-2 | NO. 81 | | NO. 82 | | NO. 83 | AEYDY |
| S008-NB1 | SEQ ID | QSISSINA | SEQ ID | IFNNGRT | SEQ ID | NIVPPLRN |
| 48-25 | NO. 90 | | NO. 91 | | NO. 92 | |
| S008-NB1 | SEQ ID | GIISSYDV | SBO ID | IGTDRWL | SEQ ID | YTFQNTVGP |
| 48-30 | NO. 99 | | NO. 100 | | NO. 101 | R |
| S008-NB1 | SEQ ID | GFTFSNYA | SEQ ID | CSASCIWT | SEQ ID | RNLDANY |
| 48-48 | NO. 108 | | NO. 109 | | NO. 110 | |
| S008-NB1 | SEQ ID | ISTFTING | SEQ ID | ISNDGTT | SEQ ID | NTIPPAGS |
| 48-5 | NO. 117 | | NO. 118 | | NO. 119 | |
| S008-NB1 | SEQ ID | GPRJINDR | SEQ ID | IDYAGST | SEQ ID | NTAPIARF |
| 48-52 | NO. 126 | | NO. 127 | | NO. 128 | |
| S008-NB1 | SEQ ID | TITISSAG | SEQ ID | TTRGGST | SEQ ID | NVVPPTY |
| 48-69 | NO. 135 | | NO. 136 | | NO. 137 | |
| S008-NB1 | SEQ ID | EGTLSSEA | SEQ ID | ISSGGST | SEQ ID | RIPNSVGP |
| 48-70 | NO. 144 | | NO. 145 | | NO. 146 | |
| S008-NB1 | SEQ ID | GFDESDSV | SEQ ID | ISSVGGS | SEQ ID | KKFGPGEY |
| 48-77 | NO. 153 | | NO. 154 | | NO. 155 | |
| S008-NB1 | SEQ ID | GSTESSYA | SEQ ID | ISSGVSA | SEQ ID | NTLPRSMPY |
| 48-8 | NO. 162 | | NO. 163 | | NO. 164 | |
| S008-NB1 | SEQ ID | GSTLSSYA | SEQ ID | ISSGGGST | SEQ ID | KSTDYGSLED |
| 48-9 | NO. 171 | | NO. 172 | | NO. 173 | S |
| S008-NB1 | SEQ ID | ARTPANAH | SEQ ID | ITWSGGIT | SEQ ID | AVDTTSAYD |
| 49-1 | NO. 180 | | NO. 181 | | NO. 182 | QLWSRGSEY |
| | | | | | | EY |
| S008-NB1 | SEQ ID | ESISVINA | SEQ ID | ISRGGST | SEQ ID | NVVPPLGS |
| 49-32 | NO. 189 | | NO. 190 | | NO. 191 | |
| S008-NB1 | SEQ ID | GSIFSRSG | SEQ ID | LTRDGHT | SEQ ID | NAIPPLGS |
| 49-35 | NO. 198 | | NO. 199 | | NO. 200 | |
| S008-NB1 | SEQ 10 | GESLDAYA | SEQ ID | ISSAGNT | SEQ ID | ARTSEGVYR |
| 49-2 | NO. 207 | | NO. 208 | | NO. 209 | GRL |
| S008-NB1 | SEQ ID | GSGFTINA | SEQ ID | ITNGGIT | SEQ ID | NIVPPVY |
| 49-20 | NO. 216 | | NO. 217 | | NO. 218 | |
| S008-NB1 | SEQ ID | GREPSTYR | SEQ ID | INWGGTAT | SEQ ID | AAGTGTTYT |
| 49-22 | NO. 225 | | NO. 226 | | NO. 227 | PQRGDAYGY |
| S008-NB1 | SEQ ID | RSISSINT | SEQ ID | LITLGGT | SEQ ID | NAVPPER |
| 49-60 | NO. 234 | | NO. 235 | | NO. 236 | |
| S008-NB1 | SEQ ID | GUESSYA | SEQ ID | ISSGGGL | SEQ ID | NVYPYTPGY |
| 49-64 | NO. 243 | | NO. 244 | | NO. 245 | |
| S008-NB1 | SEQ ID | GRTESKYA | SEQ ID | IRWIGGST | SEQ ID | AAGYQAYPE |
| 49-9 | NO. 252 | | NO. 253 | | NO. 254 | PPWEYDY |

Kabat analysis

| | | | | | | |
|---|---|---|---|---|---|---|
| S008-NB1 | SEQ ID | RIDMT | SEQ ID | RISSGGSTIYADS | SEQ ID | VPPRGS |
| 48-10 | NO. 66 | | NO. 67 | ARG | NO. 68 | |
| S008-NB1 | SEQ ID | INTMA | SEQ ID | LVTPGGGTNYAD | SEQ ID | RARVTGFAY |
| 48-13 | NO. 75 | | NO. 76 | SVKG | NO. 77 | |
| S008-NB1 | SEQ ID | SYAMT | SEQ ID | AISSGGSTNYAAS | SEQ ID | DWGNGESAE |
| 48-2 | NO. 84 | | NO. 85 | VKG | NO. 86 | YDY |
| S008-NB1 | SEQ ID | INAMD | SEQ ID | HENNGRTNYADS | SEQ ID | VPPERN |
| 48-25 | NO. 93 | | NO. 94 | VKG | NO. 95 | |
| S008-NB1 | SEQ ID | YDVVG | SEQ ID | LIGTDRWLNLGD | SEQ ID | FQNTVGPR |
| 48-30 | NO. 102 | | NO. 103 | FAKG | | |
| S008-NB1 | SEQ ID | NYAMA | SEQ ID | SCSASCIWTNYG | SEQ ID | LDANY |
| 48-48 | NO. 111 | | NO. 132 | ASVKG | NO. 13 | |
| S008-NB1 | SEQ ID | TNGWD | SEQ ID | IISNDGTTTYTDS | SEQ ID | IPPAGS |
| 48-5 | NO. 120 | | NO. 121 | | NO. 122 | |
| S008-NB1 | SEQ ID | NDRMA | SEQ ID | SIDYAGSTTYARE | SEQ ID | APTARF |
| 48-53 | NO. 129 | | NO. 130 | VKG | NO. 131 | |
| S008-NB1 | SEQ ID | SAGVS | SEQ ID | IITRGGSTNYADS | SEQ ID | VPPTY |
| 48-69 | NO 138 | | NO. 339 | VKG | NO. 140 | |
| S008-NB1 | SEQ ID | SEAMG | SEQ ID | FISSGGSTNYRDS | SEQ ID | PNSVGP |
| 48-70 | NO. 147 | | NO. 148 | VKG | NO. 149 | |
| S008-NB1 | SEQ ID | DSVMG | SEQ ID | HISSVGQSNYRDS | SEQ ID | PGPGEY |
| 48-77 | NO. 156 | | NO. 157 | | NO. 158 | |
| S008-NB1 | SEQ ID | SYAMG | SEQ ID | AISSGVSANVAD | SEQ ID | LPRSMPY |
| 48-8 | NO. 165 | | NO. 166 | SLKG | NO. 167 | |
| S008-NB1 | SEQ ID | SYAMG | SEQ ID | AISSGGGSTNYR | SEQ ID | TDYGSLEDS |
| 48-9 | NO. 174 | | NO. 175 | DSVKG | NO. 176 | |
| S008-NB1 | SEQ ID | NAHMA | SEQ ID | AITWSGGITDYA | SEQ ID | DTISAYDQL |
| 49-1 | NO. 183 | | NO. 184 | NSVKG | NO IRS | WSRQSEYEY |
| S008-NB1 | SEQ ID | INAMT | SEQ ID | LISRGGSTNYADS | SEQ ID | VPPLGS |
| 49-12 | NO. 192 | | NO. 193 | VKG | NO. 194 | |
| S008-NB1 | SEQ ID | RSGAT | SEQ ID | LLTRDGHTDYPV | SEQ 1D | IPPLGS |
| 49-45 | NO. 201 | | NO. 202 | VSVKG | NO. 203 | |
| S008-NB1 | SEQ ID | AYAIG | SEQ ID | CISSAGNTDYAD | SEQ ID | TSEGVYRGR |
| 49-2 | NO. 210 | | NO. 20 | SVKG | NO:212 | LACALYESAA |
| | | | | | | DFRS |
| S008-NB1 | SEQ ID | INAMT | SEQ ID | IITRGGITNYADS | SEQ ID | VPPVY |
| 49-20 | NO. 219 | | NO. 220 | VKG | NO. 221 | |
| S008-NB1 | SEQ ID | TYRMA | SEQ ID | AINWGGTATYYE | SEQ ID | GTGTTYTPQR |
| 49-22 | NO. 228 | | NO. 229 | DSVKG | NO. 230 | GDAYGY |
| S008-NB1 | SEQ ID | INTMT | SEQ ID | LITLGGITNYADS | SEQ ID | VPPFR |
| 49-60 | NO. 237 | | NO. 238 | VKG | NO. 339 | |
| S008-NB1 | SEQ ID | SYAMG | SEQ ID | RISSGQGLYYEDP | SEQ ID | VPYTPGY |
| 49-64 | NO. 246 | | NO. 247 | VKG | NO. 248 | |
| S008-NB1 | SEQ ID | KYAMG | SEQ ID | AIRWIGGSTYYA | SEQ ID | GYQAYPEPP |
| 49-9 | | | NO. 256 | DSVKG | NO. 237 | WEYDY |

Chothia analysis

| | | | | | | |
|---|---|---|---|---|---|---|
| S008-NB1 | SEQ ID | ISTPGRT | SEQ ID | SSGGS | SEQ ID | VPPRGS |
| 48-10 | NO. 69 | | NO. 70 | | NO. 71 | |
| S008-NB1 | SEQ ID | DDISSIY | SEQ ID | TPGGG | SEQ ID | RHRVTGRAY |
| 48-13 | NO. 78 | | NO. 79 | | NO. 80 | |
| S008-NB1 | SEQ | GSTESSY | SEQ ID | SSGGS | SEQ ID | DWGNGESAB |
| 48-2 | NO. 87 | | NO. 88 | | NO. 39 | YDY |
| S008-NB1 | SEQ ID | QSISSIN | SEQ ID | FNNGR | SEQ ID | VPPERN |
| 48-25 | NO. 96 | | NO. 97 | | NO. 98 | |
| S008-NB1 | SEQ ID | GTISSYD | SEQ ID | GTDRW | SEQ ID | FQHTVGPR |
| 48-30 | NO. 105 | | NO. 106 | | NO. 107 | |
| S008-NB1 | SEQ ID | GFTESNY | SEQ ID | SASCIW | SEQ ID | LDANY |
| 48-48 | NO. 114 | | NO. 115 | | NO. 116 | |
| S008-NB1 | SEQ ID | ISTETIN | SEQ ID | SNDGT | SEQ ID | IPPAGS |
| 48-5 | NO. 123 | | NO. 124 | | NO. 125 | |
| S008-NB1 | SEQ ID | GFRIIND | SEQ ID | DYAGS | SEQ ID | APIARF |
| 48-52 | NO. 132 | | NO. 133 | | NO. 134 | |
| S008-NB1 | SEQ ID | TITISSA | SEQ ID | TRGGS | SEQ ID | VPPTY |
| 48-69 | NO. 141 | | NO. 142 | | NO. 143 | |
| S008-NB1 | SEQ ID | EGTLSSE | SEQ ID | SSGGS | SEQ ID | PNSVGP |
| 48-70 | NO. 150 | | NO. 151 | | NO. 162 | |
| S008-NB1 | SEQ ID | GFDESDS | SEQ ID | SSVGQ | SEQ ID | FGPGEY |
| 48-77 | NO. 159 | | NO. 160 | | NO 161 | |
| S008-NB1 | SEQ ID | GSTESSY | SEQ ID | SSGVS | SEQ ID | LPRSMPY |
| 48-8 | NO. 168 | | NO. 169 | | | |
| S008-NB1 | SEQ ID | GSTLSSY | SEQ ID | SSGGGS | SEQ ID | TDYGSLEDS |
| 48-9 | NO. 177 | | NO. 178 | | NO. 179 | |
| S008-NB1 | SEQ ID | ARTFANA | SEQ ID | TWSGGI | SEQ ID | DTTSAYDQL |
| 49-1 | NO. 186 | | NO. 187 | | NO. 188 | WSRQSEYEY |
| S008-NB1 | SEQ ID | ESISVIN | SEO | SRGGS | SEQ ID | VPPLGS |
| 49-12 | NO. 195 | | NO. 196 | | NO. 197 | |
| S008-NB1 | SEQ ID | GSIFSRS | SEQ ID | TRDGH | SEQ ID | IPPLGS |
| 49-15 | NO. 204 | | NO. 205 | | NO. 206 | |
| S008-NB1 | SEQ ID | GFSLDAY | SEQ ID | SSAGN | SEQ ID | TSEGVYRGR |
| 49-2 | NO. 213 | | NO. 214 | | NO. 245 | LACALVESAA |
| | | | | | | DERS |
| S008-NB1 | SEQ ID | GSGFTIN | SEQ ID | TNGGI | SEQ ID | VPPVY |
| 49-20 | NO. 222 | | NO. 223 | | NO. 224 | |
| S008-NB1 | SEQ ID | GRPPSIY | SEQ ID | NWGGTA | SEQ ID | GTGTTYTPQR |
| 49-22 | NO. 231 | | NO. 232 | | NO. 233 | GDAYGY |
| S008-NB1 | SEQ ID | RSISSIN | SEQ ID | TLGGT | SEQ ID | VPPFR |
| 49-60 | NO. 240 | | NO. 241 | | NO. 242 | |
| S008-NB1 | SEQ ID | GIIESSY | SEQ ID | SSGGG | SEQ ID | VPYTPGY |
| 49-64 | NO. 249 | | NO. 250 | | NO. 251 | |
| S008-NB1 | SEQ ID | GRTFSKY | SEQ ID | RWIGGS | SEQ ID | GYQAYPEPP |
| 49-9 | NO. 258 | | NO. 259 | | NO. 260 | WEYDY |

### Example 2: Preparation of Nanobody, Control Antibody, EGFRvIII Protein and PepvIII Polypeptide

2.1 Expression and Purification of VHH-Fc Antibody

The VHH variable region sequence was recombined into the expression vector BI3.4-huIgG1 (from Biointron) containing a signal peptide and human IgG1 Fc (the human IgG1 Fc sequence was shown in SEQ ID NO: 6, the hinge region sequence was shown in SEQ ID NO: 7) by Taizhou Biointron Biotechnology Co., Ltd, and plasmids were prepared according to the established standard molecular biology methods. For the specific method, see Sambrook, J., Fritsch, E. F., and Maniatis, T. (1989). Molecular Cloning: A Laboratory Manual, Second Edition (Plainview, New York: Cold Spring Harbor Laboratory Press). The expression vector was transiently transfected into HEK293E cells (purchased from the Cell Bank of the Committee on Type Culture Collection of Chinese Academy of Sciences) according to the instructions of PEI (purchased from Polysciences, catalog number: 24765-1), and the transfected cells were continuously cultured at 37° C. for 5 days using FreeStyle™293 (Thermo Fisher Scientific, catalog number: 12338018), and the cell components were removed by centrifugation to obtain the culture supernatant containing the VHH-Fc antibody. The culture supernatant was loaded onto the protein A chromatography column (the protein A filler AT Protein A Diamond and the chromatography column BXK 16/26 were both purchased from Bestchrom (Shanghai) Biosciences Ltd., and the catalog numbers: AA0273 and B-1620), the column was washed with PBS phosphate buffer (pH 7.4), then washed with 20 mM PB, 1M NaCl (pH 7.2), and finally eluted with citric acid buffer (pH 3.4). The antibody with Fc label eluted from the protein A chromatography column was collected, neutralized with 1/10 volume of 1M Tris (pH 8.0), and dialyzed with PBS at 4° C. overnight, and the dialyzed protein was aseptically filtered by 0.22 micron filter membrane and then subpackaged for storage at −80° C.

2.2 Preparation of Control Antibody

C225 and 7D12 clones are antibodies that recognize human EGFR; 30D8 clone only recognizes the antibody of human EGFRvIII; The 4D5 clone is an antibody that recognizes human Her2. The heavy chain variable region (amino acid sequence SEQ ID NO. 8) and light chain variable region (amino acid sequence SEQ ID NO. 9) sequences of the C225 clone were obtained from the marketed drug Cetuximab; The heavy chain variable region (amino acid sequence SEQ ID NO. 10) of the 7D12 clone was obtained according to patent U.S. Ser. No. 10/035,856 B2 (which is incorporated herein by reference), the heavy chain variable region (amino acid sequence SEQ ID NO. 11) and the light chain variable region (amino acid sequence SEQ ID NO. 12) sequences of the 30D8 clone were obtained according to the patent U.S. Ser. No. 10/221,242 B2 (which is incorporated herein by reference); The heavy chain variable region (amino acid sequence SEQ ID NO. 13) and light chain variable region sequence (SEQ ID NO. 14) of the 4D5 clone were obtained from the marketed drug Herceptin. Plasmid construction and antibody production and purification were completed by Taizhou Biointron Biotechnology Co., Ltd. The C225 heavy chain variable region and light chain variable region were connected by three GGGGS linkers, and cloned into the BI3.4-huIgG1 vector to form the form of C225-scFv-hFc, hereinafter referred to as C225; The light chain variable region sequences of the 30D8 and 4D5 clones were cloned into the expression vector pcDNA3.4-B1HH1 containing a signal peptide and the light chain constant region of human antibody IgG1, respectively, and the heavy chain variable region sequences were cloned into the expression vector pcDNA3.4-B1HLK containing a signal peptide and the heavy chain constant region of human antibody IgG1, respectively, and thus sequences of 30D8-hIgG1 and 4D5-hIgG1, hereinafter referred to as 30D8 and 4D5, were obtained. The heavy chain antibody 7D12 variable region sequence was cloned into the expression vector BI3.4-huIgG1 containing a signal peptide and the Fc region of the human IgG1 antibody to form the form of 7D12-VHH-Fc, hereinafter referred to as 7D12. The plasmid was constructed according to the method in Example 2.1, expressed in HEK293E cells and purified, and the amino acid sequence information of the above-mentioned antibody used is shown in Table 5 below:

The negative control antibody hIgG1 was the antibody anti-hel-hIgG1 (purchased from Biointron, catalog number: B117901, hereinafter referred to as hIgG1) against Hen Egg Lysozyme chicken egg lysozyme.

TABLE 5
Specific sequence information of anti-human EGFR antibodies C225,
7D12, 30D8, anti-Her2 antibody 4D5 and human IgG1 Fc

| Designation | Sequence | |
|---|---|---|
| of sequence | number | Amino acid sequence |
| Human | SEQ ID | APELLGGPSVFLFPPKPKDTLMISRTPEVTCVVVDVSHEDPEVKFNWYVD |
| IgGI | NO: 6 | GVEVHNAKTKPREEQYNSTYRVVSVLTVLHQDWLNGKEYKCKVSNKALPA |
| Fc | | PIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHE |
| | | ALHNHYTQKSLSESPGK |
| Human | SEQ ID | EPKSADKTHTCPPCP |
| IgG1 | NO: 7 | |
| Fc hinge | | |
| region | | |
| C223 | SEQ ID | QVQLKQSGPGLVQPSQSLSITCTVSGFSLTNYGVHWVRQSPGKGLEWLGV |
| VH | NO: 8 | IWSGGNTDYNTPFTSRLSINKDNSKSQVFFKMNSLQSNDTAIYYCARALT |
| | | YYDYEFAYWGQGTLVTVSA |
| C225 | SEQ ID | DILLTQSPVILSVSPGERVSFSCRASQSIGTNIHWYQQRTNGSPRLLIKY |
| VL | NO: 9 | ASESISGIPSRFSGSGSGTDFTLSINSVESEDIADYYCQQNNNWPTTFGA |
| | | GTKLELK |
| 7D12 | SEQ ID | QVKLEESGGGSVQTGGSLRLTCAASGRTSRSYGMGWPRQAPGKEREFVSG |
| VHH | NO: 10 | ISWRGDSTGYADSVKGRFTISRDNAKNTVDLQMNSLKPEDTAIYYCAAAA |
| | | GSAWYGTLYEYDYWGQGTQVTVSS |
| 30D8 | SEQ ID | EVQLVESGQGLVKPGGSLRLSCEASGFTFSDAWMSWVRQAPGKGLEWVGR |
| VH | NO: 11 | IKSKTDGGTTDYVVPLNGRFIISRDDSRNTLYLQLNNLKTEDTAVYYCTT |
| | | VPGSYGYWGQGTLVTVSS |
| 30D8 | SEQ ID | DIVMTQSPLSLPVTPGEPASISCRSSQSLLHNKRNNYLDWFLQKPGQSPQ |
| VL | NO: 12 | LLIYLASNRASQVPDRESGGGSGTDFTLKISRVEABDYGVYYCMQAQQTP |
| | | ITFGQGTRLEIK |
| 4D5- | SEQ ID | EVQLVESGGGLVQPGGSLRLSCAASGFNIKDTYIHWVRQAPGKGLEWVAR |
| VH | NO: 13 | IYPTNGYTRYADSVKGRFTISADTSKNTAYLQMNSLRAEDTAVYYCSRWG |
| | | GDGFYAMDYWGQGTLVTVSS |
| 4D5- | SEQ ID | DIQMTQSPSSLSASVGDRVTITCRASQDVNTAVAWYQQKPGKAPKLLIYS |
| VL | NO: 14 | ASPLYSGVPSRFSGSRSGTDFTLTISSLQPEDFATYYCQQHYTTPPTFGQ |
| | | GTKVEIK |

2.3 Preparation of Human EGFRvIII-His Tag Protein

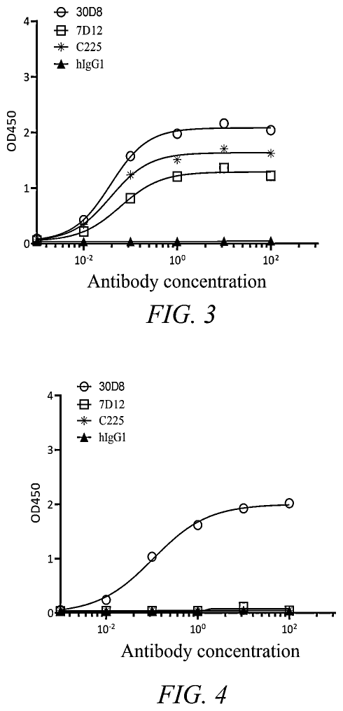

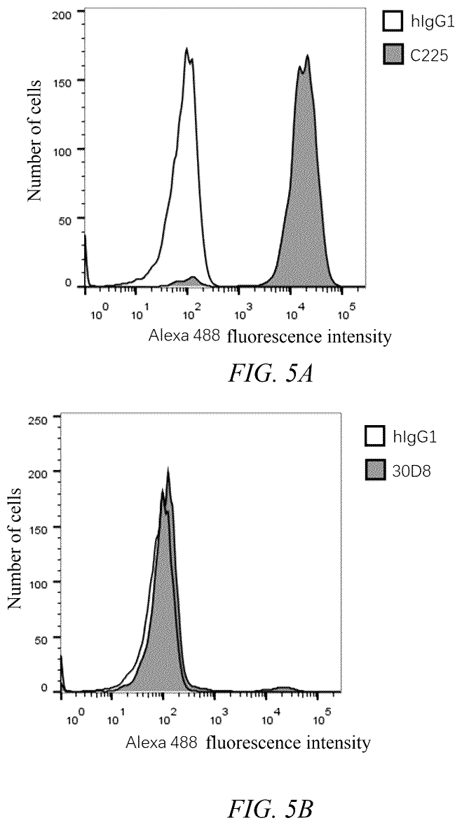

The nucleotide sequence encoding the amino acid sequence Leu25-Ser378 (SEQ ID NO: 16) of the extracellular domain (ECD) of human EGFRvIII protein (NCBI: NP_001333870.1, SEQ ID NO: 15) was cloned into the pTT5 vector (purchased from GENERAL Biosystems (Anhui) Corporation Limited) and plasmids were prepared according to the established standard molecular biology methods. The corresponding amino acid sequence information was shown in Table 6 below. For the specific method, see Sambrook, J., Fritsch, E. F., and Maniatis, T. (1989). Molecular Cloning: A Laboratory Manual, Second Edition (Plainview, New York: Cold Spring Harbor Laboratory Press). HEK293E cells (purchased from Suzhou Yiyan Biotechnology Co., Ltd.) were transiently transfected (PEI, Polysciences, catalog number: 24765-1) and FreeStyle™293 (Invitrogen, catalog number: 12338018) was used for scale-up culture at 37° C. After 6 days, the cell culture fluid was collected, centrifuged to remove cell components, and the culture supernatant containing the extracellular region of human EGFRvIII protein was obtained. The culture supernatant was loaded onto a nickel ion affinity chromatography column HisTrap™ Excel (GE Healthcare, catalog number: GE17-3712-06), and the change in the ultraviolet absorbance (A280 nm) was monitored with an ultraviolet (UV) detector. After sample loading, the nickel ion affinity chromatography column was washed with 20 mM PB, 0.5M NaCl (pH 7.4) until the ultraviolet absorbance returned to the baseline, and then gradient elutions (2%, 4%, 8%, 16%, 50%, 100%) were performed with Buffer A: 20 mM PB, 0.5M NaCl (pH 7.4) and Buffer B: 20 mM PB, 0.5M NaCl, and 500 mM imidazole. His-tagged human EGFRvIII protein eluted from the nickel ion affinity chromatography column was collected and dialyzed against PBS phosphate buffer (pH 7.4) overnight in a refrigerator at 4° C. The dialyzed protein was aseptically filtered by 0.22 micron filter membrane and then subpackaged for storage at −80° C. to obtain purified human EGFRvIII protein. The bands of interest of samples detected by SDS-PAGE reducing gel and non-reducing gel were shown in FIG. 2. The prepared EGFRvIII protein was detected by ELISA with positive control antibodies C225, 7D12, and 30D8. The detection results are shown in FIG. 3. C225, 7D12, and 30D8 could all bind to human EGFRvIII protein. The detection results are consistent with those reported in literatures, indicating that the above protein with binding activity has been prepared and obtained.

TABLE 6
Amino acid sequences of human EGFRvIII protein and
extracellular region

| Designation | Sequence | Amino acid sequence |
|---|---|---|
| of sequence | number | |
| Human | SEQ ID | MRPSGTAGAALLALLAALCPASRALEEKKGNYVVTDHGSCVRACGAD |
| EGPRVIII | NO: 15 | SYEMEEDGVRKCKKCEGPCRKVCNGIGIGEFKDSLSINATNIKHFKNCT |
| protein | | SISGDLHILPVAFRGDSFTHTPPLDPQELDILKTVKEITGFLLIQAWPENR |
| | | TDLHAFENLEIIRGRTKQHGQFSLAVVSLNITSLGLRSLKEISDGDVIISG |
| | | NKNLCYANTINWKKLFGTSGQKTKIISNRGENSCKATGQVCHALCSPE |
| | | GCWGPEPRDCVSCRNVSRGRECVDKCNLLEGEPREFVENSECIQCHPE |
| | | CLPQAMNITCTGRGPDNCIQCAHYIDGPHCVKTCPAGVMGENNTLVW |
| | | KYADAGHVCHLCHPNCTYGCTGPGLEGCPTNGPKIPSIATGMVGALLL |
| | | LLVVALGIGLFMRRRHIVRKRTLRRLLQERELVEPLTPSGEAPNQALLRI |
| | | LKETEFKKIKVLGSGAFGTVYKGLWIPEGEKVKIPVAIKELREATSPKAN |
| | | KEILDEAYVMASVDNPHVCRLLGICLTSTVQLITQLMPFGCLLDYVREH |
| | | KDNIGSQYLLNWCVQIAKGMNYLEDRRLVHRDLAARNVLVKTPQHVK |
| | | ITDEGLAKLLGAEEKEYHAEGGKVPIKWMALESILHRIYTHQSDVWSY |
| | | GVTVWELMTFGSKPYDGIPASEISSILEKGERLPQPPICTIDVYMIMVKC |
| | | WMIDADSRPKFRELIIEFSKMARDPQRYLVIQGDERMHLPSPTDSNFYR |
| | | ALMDEEDMDDVVDADEYLIPQQGFFSSPSTSRTPLLSSLSATSNNSTVA |
| | | CIDRNGLQSCPIKEDSFLQRYSSDPTGALTEDSIDDTFLPVPEYINQSVPK |
| | | RPAGSVQNPVYHNQPLNPAPSRDPHYQDPHSTAVGNPEYLNTVQPTCV |
| | | NSTFDSPAHWAQKGSHQISLDNPDYQQDFFPKEAKPNGIFKGSTAENAE |
| | | YLRVAPQSSEFIGA |
| Human | SEQ ID | LEEKKGNYVVIDAGSCVRACGADSYEMEEDGVRKCKKCEGPCRKVCNGI |
| EGPRVIII | NO: 16 | GIGEFKDSLSINATNIKHFKNCTSISGDLHILPVAFRGDSFTHTPPLDP |
| protein | | QELDILKTVKBITGFLLIQAWPENRTDLHAFENLEHIRGRTKQHGQFSLA |
| extracellular | | VVSLNITSLGLRSLKEISDGDVHISGNKNLCYANTINWKKLFGTSGQKTK |
| region | | IISNRGENSCKATGQVCHALCSPEGCWGPEPRDCVSCRNVSRGRECVD |
| | | KCNLLEGEPREFVENSECIQCHPECLPQAMNITCTGRGPDNCIQCAHYI |
| | | DGPHCVKTCPAGVMGENNTLVWKYADAGHVCHLCHPNCTYGCTGPG |
| | | LEGCPTNGPKIPS |
| Human EGPR | SEQ ID | MRPSGTAGAALLALLAALCPASRALEEKKVCQGTSNKLTQLGTFEDHF |
| protein | NO: 17 | LSLQRMFNNCEVVLGNLEITYVQRNYDLSFLKTIQEVAGYVLIALNTVE |
| | | RIPLENLQIIRGNMYYENSYALAVLSNYDANKTGLKELPMRNLQEILHG |
| | | AVRESNNPALCNVESIQWRDIVSSDFLSNMSMDPQNHLGSCQKCDPSCP |
| | | NGSCWGAGEENCQKLTKIICAQQCSGRCRGKSPSDCCHNQCAAGCTGP |
| | | RESDCLVCREFDEATCKDTCPPLMLYNPTTYQMDVNPEGKYSFGATC |
| | | VKKCPRNYVVTDHGSCVRACGADSYEMEEDGVRKCKKCEGPCRKVCNG |
| | | IGIGEFKDSLSINATNIKHFKNCTSISGDLHILPVAFRGDSFTHTPPLDP |
| | | QELDILKTVKEITGELLIQAWPENRTDLHAFENLEIIRGRTKQHGQFSLA |
| | | VVSLNITSLGLRSLKEISDGDVIISGNKNLCYANTINWKKLFGTSGQKTK |
| | | IISNRGENSCKATGQVCHALCSPEGCWGPEPRDCVSCRNVSRGRECVD |
| | | KCNLLEGEPREFVENSECIQCHPECLPQAMNITCTGRGEDNCIQCAHYI |
| | | DGPHCVKTCPAGVMGENNTLVWKYADAGHVCHLCHPNCTYGCTGPG |
| | | LEGCPTNGPKIPSIATGMVGALLLLLVVALGIGLFMRRRHIVRKKTLRRL |
| | | LQERELVEPLTPSGEAPNQALLRILKETEFKKIKVLGSGAFGTVYKGLWI |
| | | PEGEKVKIPVAIKELREATSPKANKEILDEAYVMASVDNPHVCRLLGICL |
| | | TSTVQLITQLMPPGCLLDYVREHKDENIGSQYLLNWCVQIAKGMNYLE |
| | | DRRLVHRDLAARNVLVKTPQHVKITDFGLAKLLGAEEKEYHAEGGKV |
| | | PIKWMALESILHRIYTHQSDVWYSYGVTVWELMTFGSKPYDGIPASEISSI |
| | | LEKGERLPQPPICTIDVYMMVKCWMIDADSRPKFRELIIEFSKMARDP |
| | | QRYLVIQGDERMHLPTDSNFYRALMDEEDMDDVVDADEYLIPQQGFFS |
| | | SPSTSRTPLLSSLSATSNNSTVACIDRNGLQSCPIKEDSFLQRYSSDPT |
| | | GALTEDSIDDTFLPVPEYINQSVPKRPAGSVQNPVYHNQPLNPAPSRDPH |
| | | YQDPHSTAVGNPEYLNTVQPTCVNSTFDSPAHWAQKGSHQISLDNPDY |
| | | QQDFFPKEAKPNGIFKGSTAENAEYLRVAPQSSEFIGA |
| Monkey EGFR | SEQ ID | MRPSGTAGAALLALLAALCPASRALEEKKVCQGTSNKLTQLGTFEDHF |
| protein | NO: 18 | LSLQRMFNNCEVVLGNLEITYVQRNYDFSFLKTIQEVAGYVLIALNTVE |
| | | RIPLENLQIIRGNMYYENSYALAVLSNYDANKTGLKELPMRNLQEILHG |
| | | AVRFSNNPALCNVESIQWRDIVSSEFLSNMSMDFQNHLGSCQKCDPSCP |
| | | NGSCWGAGEENCQKLTKIICAQQCSGRCRGKSPSDCCHNQCAAGCTGP |
| | | RESDCLVCRKFRDEATCKDTCPPLMLYNPTTYQMDVNPEGKYSFGATC |
| | | VKKCPRNYVVTDHGSCVRACGADSYEMEEDGYRKCKKCEGPCRKVCNG |
| | | IGIGEFKDTLSINATNIKHFKNCTSISGDLHILPVAFRGDSFTHTPPLDP |
| | | QELDILKTVKEITGFLLIQAWPENRTDLHAFENLEIIRGRTKQHGQFSLA |
| | | VVSLNITSLGLRSLKEISDGDVIISGNKNLCYANTINWKKLFGTSSQKTK |
| | | IISNRGENSCKATGQVCHALCSPEGCWGPEPRDCVSCQNVSRGRECVD |
| | | KCNILEGEPREFVENSECIQCHPECLPQVMNITCTGRGPDNCIQCAHYID |
| | | GPHCVKTCPAGVMGENNTLVWKYADAGHYCHLCHPNCTYGCTGPGL |
| | | EGCARNGPKIPSIATGMVGALLLLLVVALGIGLFMRRRHIVRKRTLRRL |
| | | LQERELVEPLTPSGEAPN |
| | | QALLRILKETEFKKIKVLGSGAFGTVYKGLWIPEGEKVKIPVAIKELREA |
| | | TSPKANKEILDEAYVMASVDNPHVCRLLGICLTSTVQLITQLMPFGCLL |
| | | DYVREHKDNIGSQYLLNWCVQIAKGMNYLEDRRLVHRDLAARNVLV |
| | | KTPQHVKITDFGLAKLLGAEEKEYHAEGGKVPIKWMALESILBRIYTH |
| | | QSDVWSYGVTVWELMTFGSKPYDGIPASEISSILEKGERLPQPPICTIDV |
| | | YMIMYKCWMIDADSRPKFRELIIEFSKMARDPQRYLVIQGDERMHLPSP |
| | | TDSNFYRALMDEEDMDDVVDADEYLIPQQGFFSSPSTSRTPLLSSLSAT |
| | | SNNSTVACIDRNGLQSCPIKEDSFLQRYSSDPTGALTEDSIDDTFLPVPEY |
| | | INQSVPKRPAGSVQNPVYHNQPLNPAPSRDPHYQDPHSTAVGNPEYLNT |
| | | VQPTCVNSTFDSPAHWAQKGSHQISLDNPDYQQDPFPKEAKPNGIFKGS |
| | | TAENAEYLRVAPQSSEFIGA |

2.4 Preparation of pepvIII Polypeptide

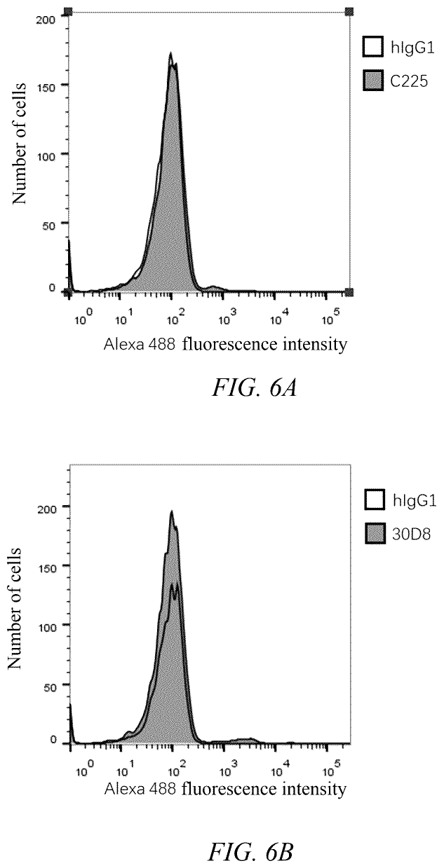

GL Biochem Ltd. was entrusted with the production of Leu25-His37 polypeptide pepvIII (LEEKKGNYVVTDH) from human EGFRvIII (NCBI: NP_001333870.1, SEQ ID NO: 15). The prepared pepvIII polypeptide described above was detected by ELISA with positive control antibodies recognizing different epitopes, and the detection results are shown in FIG. 4. C225 and 7D12 could not bind to the pepvIII polypeptide, but 30D8 could bind to the pepvIII polypeptide. The detection results are consistent with those reported in literatures, indicating that the above polypeptide with binding activity has been prepared and obtained.

### Example 3 Identification of Endogenous Expression Cell Line and Preparation of Overexpression Cell Line

3.1 Identification of Cell Line Expressing EGFR Endogenously

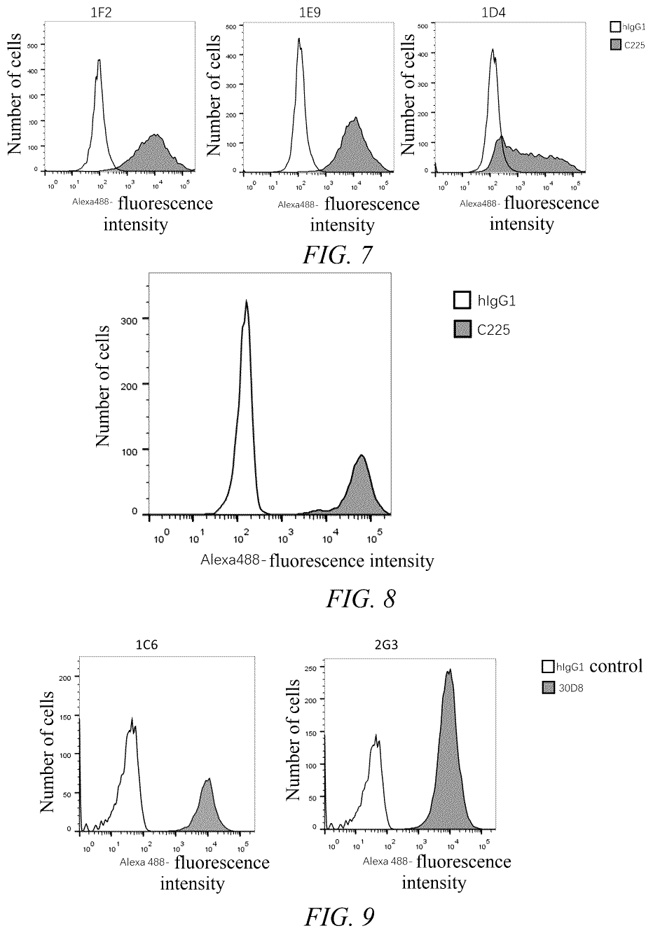

A431 cells (purchased from Cell bank of Chinese Academy of Sciences, catalog number: TCHu188) were scale-up cultured in T-175 cell culture flasks to the logarithmic growth phase, the supernatant of the medium was discarded by centrifugation, and the cell pellet was washed 2 times with PBS. The C225 and 30D8 antibodies were used as primary antibodies, Alexa488-labeled secondary antibody (purchased from Invitrogen, catalog number: A11013) was used and FACS (FACS Canto™, purchased from BD company) was used for detection and result analysis. The analysis results are shown in Table 7 and FIGS. 5A-5B. The results show that A431 cells can bind to C225, but not bind to 30D8, and A431 cells are positive cells screened by EGFR antibody.

TABLE 7
FACS detection results of the endogenous cell line A431 cells

| | Designation | Average fluorescence intensity of A431 cell | |
|---|---|---|---|
| | of | IgG subtype | Positive |
| No. | antibody | control | antibody |
| 1 | C225 | 74 | 14925 |
| 2 | 30D8 | 74 | 528 |

3.2 Identification of Cell Line without Endogenous EGFR Expression

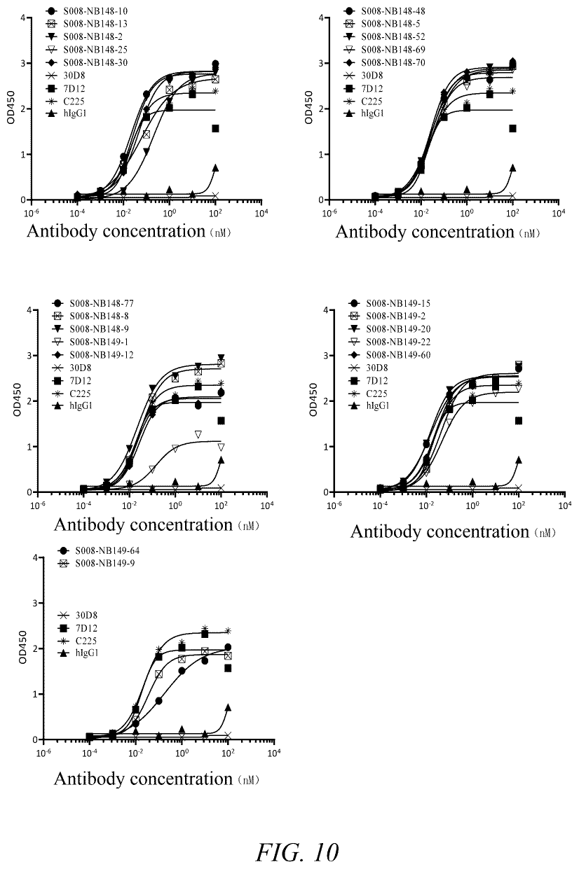

MCF-7 cells (purchased from Cell bank of Chinese Academy of Sciences, catalog number: TCHu74) were scale-up cultured in T-175 cell culture flasks to the logarithmic growth phase, the supernatant of the medium was discarded by centrifugation, and the cell pellet was washed 2 times with PBS. The C225 and 30D8 antibodies were used as primary antibodies, Alexa488-labeled secondary antibody was used and FACS (FACS Canto™, purchased from BD company) was used for detection and result analysis. The analysis results are shown in Table 8 and FIGS. 6A-6B. The results show that MCF-7 cells do not bind to C225 and 30D8, and MCF-7 cells are negative cells screened by EGFR antibody.

TABLE 8
FACS detection results of the endogenous cell line MCF-7 cells

| | | Average fluorescence intensity | |
|---|---|---|---|
| | | of MCF-7 cells | |
| | Designation | IgG subtype | Positive |
| No. | of antibody | control | antibody |
| 1 | C225 | 79 | 140 |
| 2 | 30D8 | 79 | 86 |

3.3 Preparation of Monoclonal Cell Line of CHO-K1 Cells Stably Transfected with Human EGFR

The nucleotide sequence encoding human EGFR (NCBI: NP_005219, SEQ ID NO: 17) was cloned into the pcDNA3.1 vector by GENERAL Biosystems (Anhui) Corporation Limited and a plasmid was prepared. Plasmid transfection (Lipofectamine® 3000 Transfection Kit, purchased from Invitrogen, catalog number: L3000-015) was performed on CHO-K1 cell line (purchased from the Cell Bank of the Committee on Type Culture Collection of Chinese Academy of Sciences), and then the transfected cells were selectively incubated for 2 weeks in DMEM/F12 medium containing 10 μg/ml of puromycin and 10% (w/w) of fetal bovine serum. The anti-EGFR antibody C225 was used to sort the positive monoclonal cells into a 96-well plate on flow cytometer FACS AriaII (BD Biosciences) and the plate was placed in a cell incubator at 37° C. and 5% (v/v) CO₂ for cell culture. Some wells containing monoclonal cells were selected for amplification after approximately 2 weeks. The amplified clones were screened by flow cytometry using the C225 antibody as the primary antibody. The monoclonal cell line with better growth and higher fluorescence intensity was selected for further scale-up culture and then freezed in liquid nitrogen.

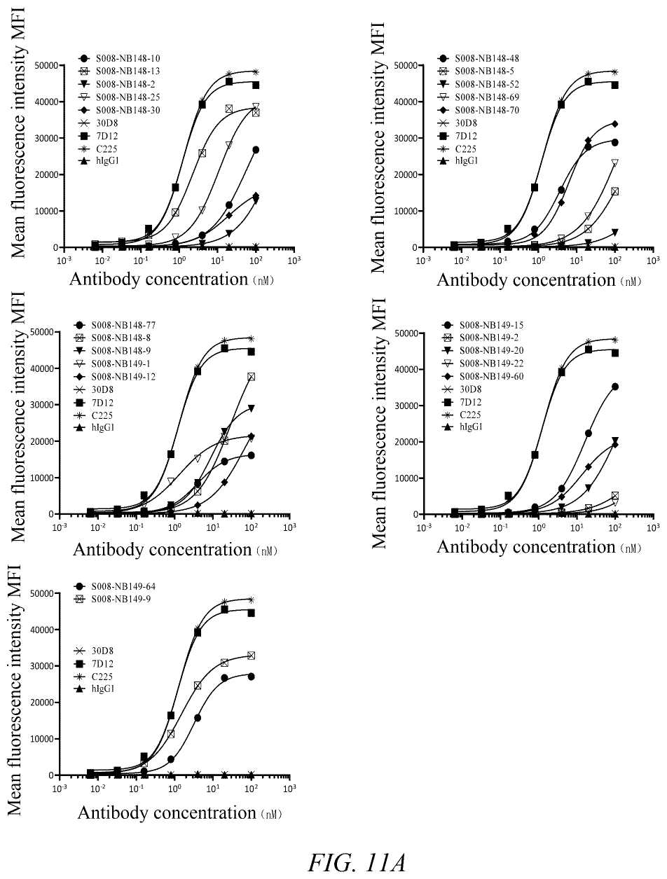

The specific selection results were shown in Table 9 and FIG. 7, and the IgG subtype negative control was hIgG1 control. The results in Table 9 and FIG. 7 show that a series of CHO-K1 monoclonal cell lines with positive expression of EGFR have been prepared, CHO-K1-human EGFR1F2, CHO-K1-human EGFR1E9 and CHO-K1-human EGFR1D4 are monoclonal cell lines with high level of EGFR expression.

TABLE 9
FACS detection results of CHO-K1 cell line stably transfected with
human EGFR protein

| | | Average fluorescence | |
|---|---|---|---|
| | Designation of stably | intensity of cells | |
| No. | transfected cell line clone | IgG subtype control | Antibody C225 |
| 1 | CHO-K1-Human EGFR 1D4 | 1561 | 168073 |
| 2 | CHO-K1-Human EGFR 1E9 | 1561 | 197969 |
| 3 | CHO-KT-Human EGFR 1F2 | 1561 | 143666 |

3.4 Preparation of HEK293T Cell Line Stably Transfected with Monkey EGFR

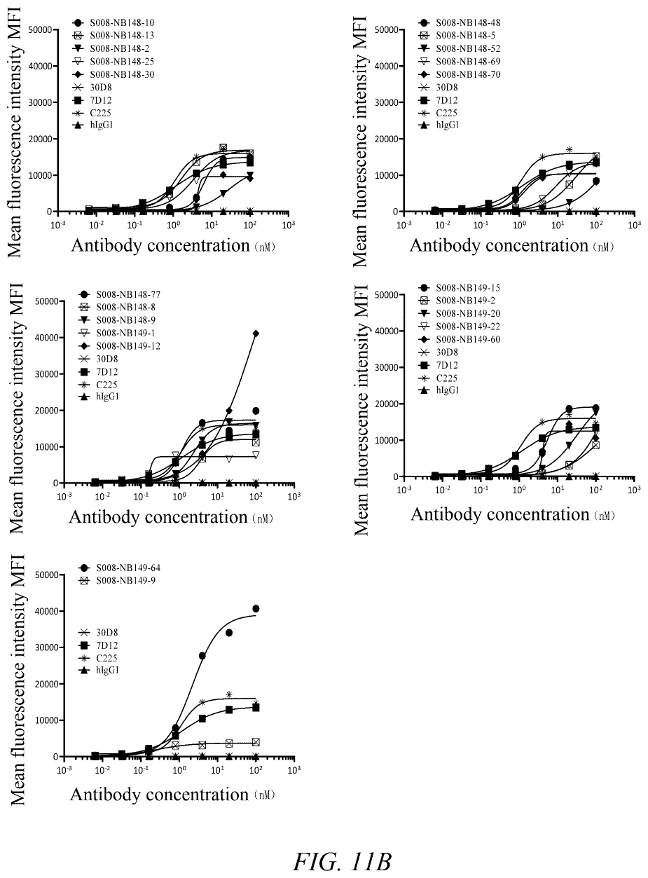

The nucleotide sequence encoding the full-length amino acid sequence of cynomolgus monkey EGFR (hereinafter referred to as monkey EGFR) (NCBI: XP_005549616.1, SEQ ID NO: 18) was cloned into the pcDNA3.1 vector (completed by GENERAL Biosystems (Anhui) Corporation Limited, catalog number: GNHa7) and a plasmid was prepared. After plasmid transfection of the HEK293T cell line with FUGENE® HD (Promega, catalog number: #E2311), the transfected cells were selectively cultured in DMEM medium containing 10 μg/ml puromycin and 10% (w/w) fetal bovine serum for 2 weeks, subcloned in 96-well culture plates by limited dilution method, and cultured in a 37° C., 5% (v/v) CO₂ cell incubator. After about 2 weeks, some wells contained polyclones were selected and amplification was performed into 6-well plates. The amplified clones were screened by EGFR antibody C225 with monkey cross activity by flow cytometry analysis, the cell line with better growth and higher fluorescence intensity was selected for further scale-up culture and then freezed and stored in liquid nitrogen. FIG. 8 is the result of flow cytometry analysis of the HEK293T cell line detected by C225 antibody. The result shows that the cell line that shows a single positive cell peak overexpressing monkey EGFR after puromycin screening could be used to detect the cross-activity of antibodies.

3.5 Preparation of CHO-K1 Cell Line Stably Transfected with Human EGFRvIII

The nucleotide sequence encoding human EGFRvIII (NCBI: NP_001333870.1, SEQ ID NO: 15) was cloned into the pcDNA3.1 vector by GENERAL Biosystems (Anhui) Corporation Limited and a plasmid was prepared. According to the method described in 3.3, the monoclonal amplification of the CHO-K1-EGFRvIII cell line was completed, and the amplified clones were screened by flow cytometry using 30D8 as the primary antibody. The monoclonal cell line with better growth and higher fluorescence intensity was selected for further scale-up culture and then freezed in liquid nitrogen.

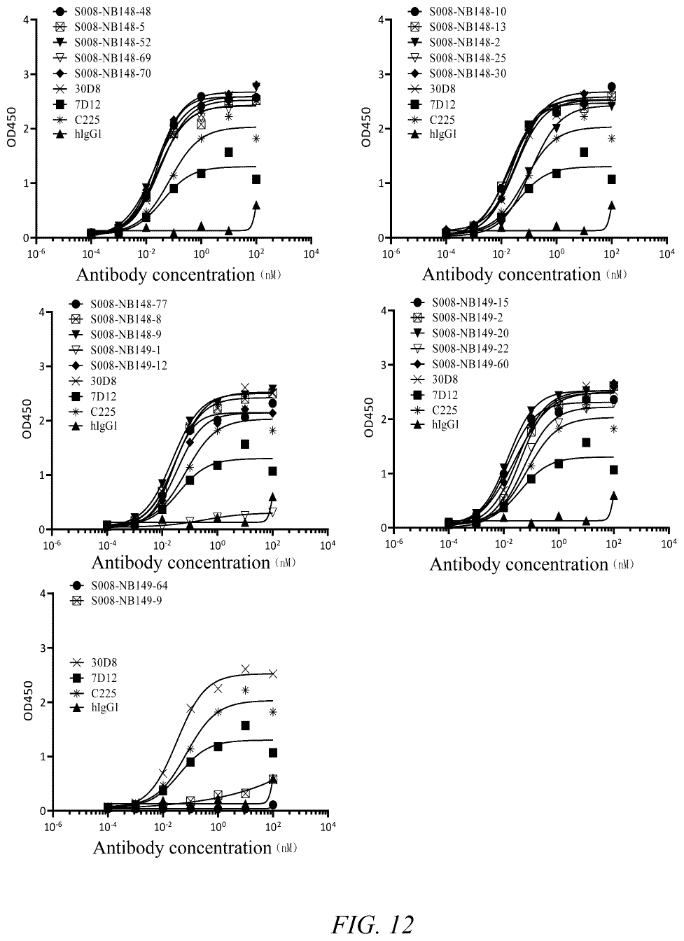

The specific selection results were shown in Table 10 and FIG. 9, and the IgG subtype control was hIgG1 control. The results in Table 10 and FIG. 9 indicate that a series of CHO-K1 monoclonal cell lines positively expressing human EGFRvIII have been prepared, and CHO-K1-EGFRvIII1C6 and CHO-K1-EGFRvIII2G3 are monoclonal cell lines with high level of EGFRvIII expression.

TABLE 10
FACS detection results of CHO-K1 cell line stably transfected with
human EGFRvIII protein

| | Designation of stably | Average fluorescence intensity of cells | |
|---|---|---|---|
| | transfected | IgG subtype | Antibody |
| No. | cell line clone | controll | 30D8 |
| 1 | CHO-K1-EGFRvIII 1C6 | 35.4 | 10048 |
| 2 | CHO-K1-EGFRvIII 2G3 | 35.4 | 10220 |

### Example 4 Identification of the Binding Ability of VHH-Fc Antibodies to Human EGFR

4.1 Binding of VHH-Fc Antibodies to EGFR Protein Detected by Enzyme-Linked Immunosorbent Assay (ELISA)

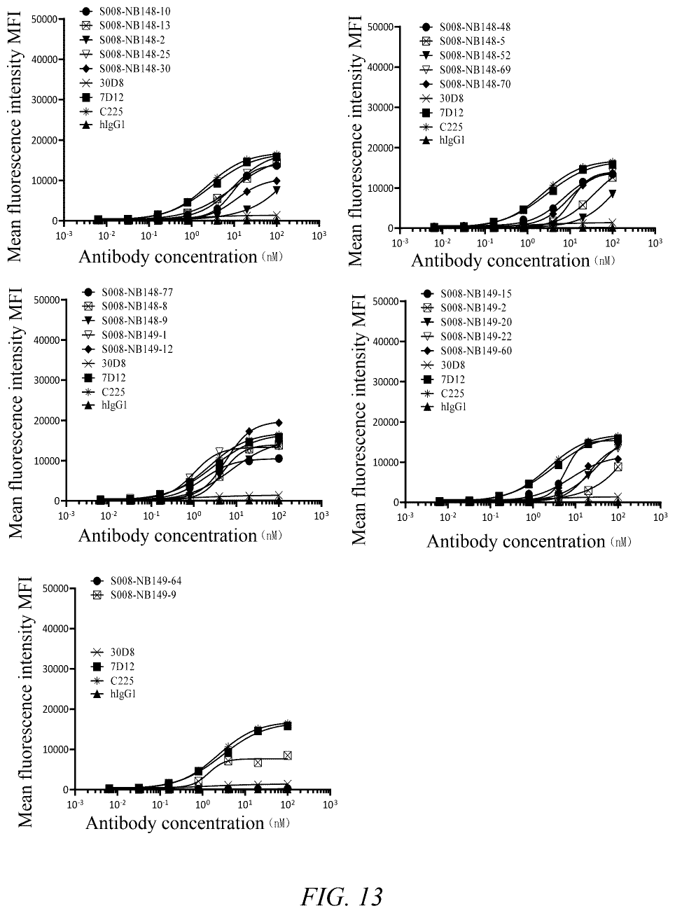

In order to detect the binding activity of VHH-Fc to EGFR protein, the human EGFR protein (purchased from Acro, catalog number: EGR-H5222) was diluted with PBS to a final concentration of 1 μg/mL, and then added to a 96-well ELISA plate at 50 μl/well. The plate was sealed with plastic film and incubated overnight at 4° C., the plate was washed 2 times with PBS the next day, and then a blocking solution [PBS+2% (w/w) BSA] was added for blocking at room temperature for 2 hours. The blocking solution was poured off, and 100 nM of serially diluted the VHH-Fc antibody or a negative control antibody was added at 50 μl/well. After incubation at 37° C. for 2 hours, the plate was washed 3 times with PBS. HRP (horseradish peroxidase)-labeled secondary antibody (purchased from Sigma, catalog number: A0170) was added, and incubated at 37° C. for 2 hours, and the plate was washed 5 times with PBS. TMB substrate was added at 50 μl/well, and incubated at room temperature for 30 minutes, then a stop solution (1.0 N HCl) was added at 50 μl/well. An ELISA plate reader (Multimode Plate Reader, EnSight, purchased from Perkin Elmer) was used to read the OD450 nm value, and the ELISA results of the VHH-Fc and the human EGFR are shown in FIG. 10 and Table 11, wherein the IgG control is hIgG1; 7D12 and C225 are positive controls for EGFR protein. The results indicated that all the VHH-Fc antibodies binded to human EGFR protein at ELISA level.

TABLE 11
Binding reaction of VHH-Fc antibodies with
human EGFR protein detected by ELISA

| | OD450 | | | | | | | |
|---|---|---|---|---|---|---|---|---|
| | Antibody concentration (nM) | | | | | | | |
| Designation of antibody | 100 | 10 | 1 | 0.1 | 0.01 | 0.001 | 0.0001 | 0 |
| S008-NB148-10 | 2.99 | 2.70 | 2.68 | 2.32 | 0.95 | 0.20 | 0.07 | 0.05 |
| S008-NB148-13 | 2.65 | 2.50 | 2.42 | 1.44 | 0.77 | 0.18 | 0.06 | 0.05 |
| S008-NB148-2 | 2.80 | 2.61 | 2.16 | 1.05 | 0.19 | 0.06 | 0.05 | 0.05 |
| S008-NB148-25 | 2.91 | 2.70 | 2.60 | 2.26 | 0.78 | 0.16 | 0.06 | 0.07 |
| S008-NB148-30 | 2.91 | 2.75 | 2.64 | 1.99 | 0.60 | 0.18 | 0.12 | 0.05 |
| S008-NB148-48 | 2.96 | 2.64 | 2.69 | 2.23 | 0.79 | 0.17 | 0.09 | 0.06 |
| S008-NB148-5 | 2.94 | 2.82 | 2.61 | 2.13 | 0.76 | 0.15 | 0.06 | 0.06 |
| S008-NB148-52 | 2.94 | 2.91 | 2.64 | 2.24 | 0.85 | 0.18 | 0.07 | 0.05 |
| S008-NB148-69 | 2.86 | 2.59 | 2.49 | 2.11 | 0.72 | 0.14 | 0.06 | 0.06 |
| S008-NB148-70 | 3.05 | 2.79 | 2.81 | 2.36 | 0.84 | 0.17 | 0.06 | 0.05 |
| S008-NB148-77 | 2.18 | 1.90 | 2.07 | 1.78 | 0.67 | 0.13 | 0.06 | 0.35 |
| S008-NB148-8 | 2.83 | 2.65 | 2.50 | 2.08 | 0.70 | 0.14 | 0.06 | 0.06 |
| S008-NB148-9 | 2.94 | 2.76 | 2.55 | 2.27 | 0.95 | 0.21 | 0.07 | 0.05 |
| S008-NB149-1 | 0.98 | 1.26 | 0.95 | 0.49 | 0.16 | 0.06 | 0.03 | 0.04 |
| S008-NB149-12 | 2.22 | 1.95 | 2.06 | 1.70 | 0.57 | 0.11 | 0.04 | 0.07 |
| S008-NB149-15 | 2.71 | 2.47 | 2.35 | 2.14 | 1.05 | 0.23 | 0.09 | 0.24 |
| S008-NB149-2 | 2.79 | 2.49 | 2.35 | 1.86 | 0.51 | 0.10 | 0.05 | 0.05 |
| S008-NB149-20 | 2.75 | 2.42 | 2.39 | 2.24 | 1.07 | 0.23 | 0.08 | 0.06 |
| S008-NB149-22 | 2.27 | 2.17 | 1.96 | 1.52 | 0.42 | 0.09 | 0.04 | 0.05 |
| S008-NB149-60 | 2.71 | 2.45 | 2.33 | 2.08 | 0.76 | 0.15 | 0.06 | 0.05 |
| S008-NB149-64 | 2.03 | 1.73 | 1.51 | 0.85 | 0.35 | 0.07 | 0.05 | 0.05 |
| S008-NB149-9 | 1.84 | 1.94 | 1.77 | 1.44 | 0.43 | 0.09 | 0.06 | 0.13 |
| 30D8 | 0.09 | 0.07 | 0.05 | 0.05 | 0.05 | 0.06 | 0.05 | 0.05 |
| 7D12 | 1.57 | 2.32 | 2.02 | 1.82 | 0.66 | 0.13 | 0.06 | 0.05 |
| C225 | 2.39 | 2.44 | 2.13 | 1.98 | 0.73 | 0.15 | 0.06 | 0.06 |
| hIgG1 | 0.71 | 0.13 | 0.23 | 0.09 | 0.19 | 0.08 | 0.05 | 0.07 |

4.2 The Binding of Antibody to Different EGFR Expressing Cells Detected by Flow Cytometry (FACS)

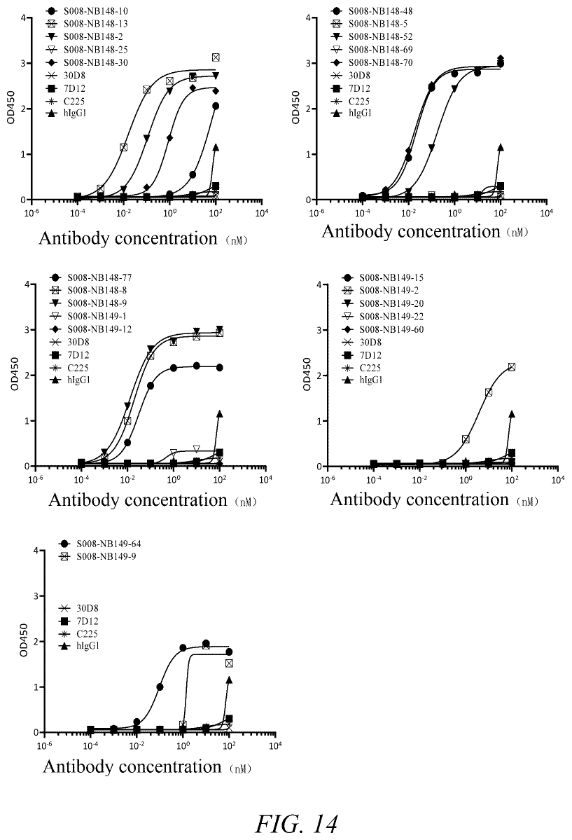

The required cells were scale-up cultured in a T-75 cell culture flask to the logarithmic growth phase. For the adherent cells A431, MCF-7, CHO-K1 and HEK293T, the medium was aspirated, the cells were washed 2 times with PBS buffer, and then digested with trypsin. After the digestion was terminated, the cells were washed 2 times with PBS buffer. After counting the cells in the previous step, the cell pellet was resuspended with [PBS+2% (w/w) FBS] blocking solution to 4×10⁶ cells/ml, and added to a 96-well FACS reaction plate at 50 μl/well, and then the VHH-Fc antibody test sample was added at 50 μl/well, and incubated on ice for 1 hours. The mixture was centrifuged and washed 3 times with PBS buffer, Alexa Flour 488-labeled secondary antibody (purchased from Invitrogen, catalog number: A-11013) was added at 50 μl/well, and incubated on ice for 1 hour. The obtained mixture was centrifuged and washed 5 times with PBS, and FACS (FACS Canto™, purchased from BD Company) was used for detection and result analysis. Data analysis was performed by software (CellQuest) to obtain the mean fluorescence intensity (MFI) of the cells. And then software (GraphPad Prism8) was used for analysis, data fitting, and EC50 value calculation. The analysis results are shown in Table 12 and FIGS. 11A-11B, wherein the IgG control is hIgG1; 7D12 and C225 are positive controls for EGFR protein. The results showed that the VHH-Fc antibodies all can bind to the human EGFR protein on the surface of A431 cells and CHO-K1-human EGFR 1D4 cells.

TABLE 12
Binding reaction of VHH-Fc antibodies with A431 cells and CHO-K1-
human EGFR1D4 cells detected by FACS

| | A431 | | CHO-K1-human EGFR 1D4 | |
|---|---|---|---|---|
| | Maximum | | Maximum | |
| | average | | average | |
| | fluorescence | | fluorescence | |
| Designation of | intensity | EC50 | intensity | EC50 |
| antibody | Max MFI | nM | Max MFI | nM |
| S008-NB148-10 | 26777 | 57.80 | 14785 | 6.20 |
| S008-NB148-13 | 36967 | 2.24 | 15941 | 1.63 |
| S008-NB148-2 | 12683 | Poor fit | 9894 | 28.08 |
| S008-NB148-25 | 38536 | 10.87 | 15366 | 3.32 |
| S008-NB148-30 | 14199 | 19.34 | 9108 | 4.33 |
| S008-NB148-48 | 28813 | 3.61 | 8369 | 1.17 |
| S008-NB148-5 | 15376 | 151.20 | 15161 | 30.28 |
| S008-NB148-52 | 4026 | Poor fit | 8083 | 219.00 |
| S008-NB148-69 | 23090 | 115.90 | 13181 | 8.89 |
| S008-NB148-70 | 33872 | 6.28 | 8470 | 1.35 |
| S008-NB148-77 | 16112 | 3.96 | 19856 | 1.16 |
| S008-NB148-8 | 37698 | 28.81 | 11208 | 3.60 |
| S008-NB148-9 | 28927 | 8.77 | 15789 | 2.37 |
| S008-NB149-1 | 20690 | 1.34 | 7633 | Poor fit |
| S008-NB149-12 | 21367 | 69.49 | 41102 | 87.71 |
| S008-NB149-15 | 35227 | 16.41 | 18798 | 5.38 |
| S008-NB149-2 | 5099 | 233.80 | 8676 | 106.10 |
| S008-NB149-20 | 20252 | 127.00 | 17590 | 32.27 |
| S008-NB149-22 | 3193 | Poor fit | 12190 | Poor fit |
| S008-NB149-60 | 19238 | 13.48 | 10518 | 4.05 |
| S008-NB149-64 | 27098 | 3.23 | 40657 | 2.21 |
| S008-NB149-9 | 32862 | 1.50 | 3958 | 0.26 |
| 30D8 | 143 | Negative | 88 | Negative |
| 7D12 | 44548 | 1.22 | 13445 | 1.16 |
| C225 | 48111 | 1.30 | 14707 | 1.09 |
| hIgG1 | 159 | Negative | 89 | Negative |

Remark: “Poor fit” indicates that EC50 value could not be calculated.

### Example 5: Identification of Binding Ability of VHH-Fc Antibodies to Human EGFRvIII

5.1 Binding of VHH-Fc Antibodies to EGFRvIII Protein Detected by Enzyme-Linked Immunosorbent Assay (ELISA)

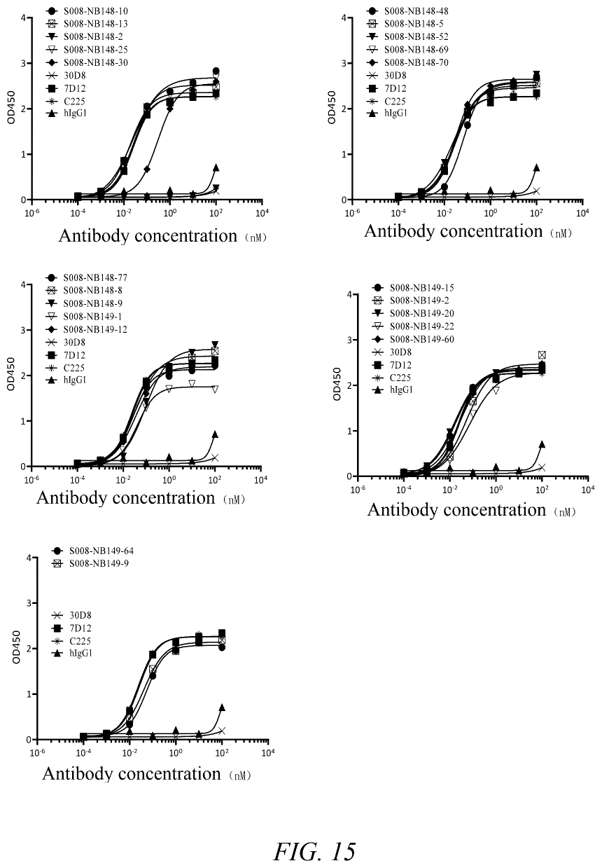

In order to detect the binding activity of the VHH-Fc to the EGFRvIII protein, the purified human EGFRvIII protein obtained in Example 2 was diluted with PBS to a final concentration of 1 μg/mL, and then added to a 96-well ELISA plate at 50 μl/well. According to the experimental method described in 4.1, the binding activity of the VHH-Fc antibodies to the human EGFRvIII protein was detected. The experimental results are shown in FIG. 12 and Table 13. Wherein the IgG control is hIgG1; 30D8, 7D12, and C225 are positive controls for the EGFRvIII protein. The data in the table are OD450 nm values. The results showed that the antibody 5008-NB149-64 had no binding activity to the EGFRvIII protein in the ELISA experiment, the antibodies S008-NB149-1 and 5008-NB149-9 had weak binding to the EGFRvIII protein in the ELISA experiment, and other VHH-Fc antibodies had good binding to the human EGFRvIII at ELISA level.

TABLE 13
Binding reaction of VHH-Fc antibodies with
human EGFRvIII protein detected by ELISA

| | OD450 | | | | | | | |
|---|---|---|---|---|---|---|---|---|
| | Antibody concentration(nM) | | | | | | | |
| Designation of antibody | 100 | 10 | 1 | 0.1 | 0.01 | 0.001 | 0.0001 | 0 |
| S008-NB148-10 | 2.77 | 2.47 | 2.29 | 2.06 | 0.90 | 0.21 | 0.08 | 0.07 |
| S008-NB148-13 | 2.60 | 2.38 | 2.32 | 2.05 | 0.94 | 0.21 | 0.08 | 0.10 |
| S008-NB148-2 | 2.41 | 2.41 | 2.00 | 1.21 | 0.25 | 0.08 | 0.06 | 0.06 |
| S008-NB148-25 | 2.71 | 2.41 | 2.37 | 2.06 | 0.77 | 0.17 | 0.07 | 0.08 |
| S008-NB148-30 | 2.78 | 2.64 | 2.38 | 1.98 | 0.71 | 0.24 | 0.15 | 0.08 |
| S008-NB148-48 | 2.57 | 2.54 | 2.59 | 2.06 | 0.75 | 0.18 | 0.09 | 0.07 |
| S008-NB148-5 | 2.52 | 2.47 | 2.08 | 1.92 | 0.75 | 0.16 | 0.07 | 0.07 |
| S008-NB148-52 | 2.76 | 2.48 | 2.32 | 2.05 | 0.91 | 0.21 | 0.08 | 0.06 |
| S008-NB148-69 | 2.53 | 2.36 | 2.20 | 1.87 | 0.73 | 0.16 | 0.07 | 0.08 |
| S008-NB148-70 | 2.80 | 2.63 | 2.44 | 2.16 | 0.87 | 0.19 | 0.08 | 0.07 |
| S008-NB148-77 | 2.32 | 2.06 | 2.01 | 1.82 | 0.62 | 0.13 | 0.05 | 0.04 |
| S008-NB148-8 | 2.50 | 2.40 | 2.22 | 1.86 | 0.69 | 0.15 | 0.07 | 0.07 |
| S008-NB148-9 | 2.58 | 2.47 | 2.32 | 1.99 | 0.84 | 0.21 | 0.08 | 0.06 |
| S008-NB149-1 | 0.31 | 0.25 | 0.22 | 0.14 | 0.05 | 0.05 | 0.05 | 0.04 |
| S008-NB149-12 | 2.14 | 2.21 | 1.95 | 1.60 | 0.53 | 0.10 | 0.05 | 0.04 |
| S008-NB149-15 | 2.36 | 2.36 | 2.12 | 2.01 | 0.99 | 0.24 | 0.10 | 0.10 |
| S008-NB149-2 | 2.61 | 2.39 | 2.27 | 1.77 | 0.53 | 0.12 | 0.07 | 0.10 |
| S008-NB149-20 | 2.55 | 2.52 | 2.43 | 2.15 | 1.10 | 0.26 | 0.10 | 0.07 |
| S008-NB149-22 | 2.30 | 2.19 | 1.93 | 1.47 | 0.38 | 0.08 | 0.04 | 0.04 |
| S008-NB149-60 | 2.66 | 2.39 | 2.15 | 1.90 | 0.84 | 0.17 | 0.08 | 0.07 |
| S008-NB149-64 | 0.11 | 0.04 | 0.04 | 0.04 | 0.04 | 0.04 | 0.04 | 0.04 |
| S008-NB149-9 | 0.58 | 0.32 | 0.29 | 0.18 | 0.07 | 0.06 | 0.06 | 0.11 |
| 30D8 | 2.52 | 2.61 | 2.25 | 1.89 | 0.69 | 0.15 | 0.07 | 0.07 |
| 7D12 | 1.07 | 1.57 | 1.18 | 0.90 | 0.37 | 0.11 | 0.06 | 0.06 |
| C225 | 1.82 | 2.22 | 1.82 | 1.14 | 0.47 | 0.13 | 0.06 | 0.06 |
| hIgG1 | 0.60 | 0.13 | 0.22 | 0.09 | 0.19 | 0.08 | 0.07 | 0.07 |

5.2 The Binding of Antibody to EGFRvIII Expressing Cells Detected by Flow Cytometry (FACS)

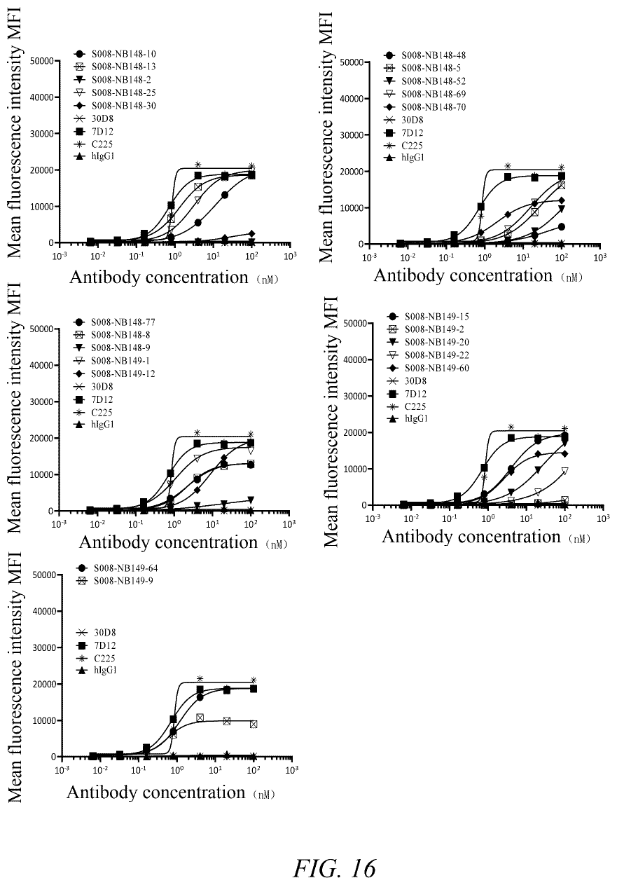

According to the experimental and analytical method of the flow cytometry experiment described in 4.2, the binding ability of the VHH-Fc antibodies to the EGFRvIII protein on the surface of the CHO-K1 cell line was analyzed. The analysis results are shown in Table 14 and FIG. 13, wherein the IgG control is hIgG1; 30D8, 7D12, and C225 are positive controls for the EGFRvIII protein. The results showed that the antibody S008-NB149-64 had no binding activity to the EGFRvIII protein on the surface of CHO-K1-EGFRvIII 1C6, and other VHH-Fc antibodies could bind to the human EGFRvIII protein on the surface of CHO-K1-EGFRvIII 1C6 cells.

TABLE 14
Binding reaction of VHH-Fc antibodies with CHO-K1-EGFRvIII 1C6
cells detected by FACS

| | CHO-K1-EGFRvIII 1C6 | |
|---|---|---|
| | Maximum average | |
| | fluorescence intensity | EC50 |
| Designation of antibody | Max MFI | nM |
| S008-NB148-10 | 13686 | 9.37 |
| S008-NB148-13 | 14268 | 9.83 |
| S008-NB148-2 | 7544 | Poor fit |
| S008-NB148-25 | 15607 | 9.88 |
| S008-NB148-30 | 9910 | 11.19 |
| S008-NB148-48 | 13429 | 6.87 |
| S008-NB148-5 | 12625 | 37.37 |
| S008-NB148-52 | 8471 | 173.30 |
| S008-NB148-69 | 13800 | 11.09 |
| S008-NB148-70 | 13730 | 9.50 |
| S008-NB148-77 | 10524 | 1.63 |
| S008-NB148-8 | 13882 | 4.73 |
| S008-NB148-9 | 14147 | 7.60 |
| S008-NB149-1 | 13612 | 1.00 |
| S008-NB149-12 | 19394 | 5.74 |
| S008-NB149-15 | 15170 | 6.00 |
| S008-NB149-2 | 8865 | Poor fit |
| S008-NB149-20 | 13987 | 38.43 |
| S008-NB149-22 | 13442 | 20.80 |
| S008-NB149-60 | 10757 | 7.76 |
| S008-NB149-64 | 265 | Negative |
| S008-NB149-9 | 8482 | 1.38 |
| 30D8 | 1384 | 0.98 |
| 7D12 | 15844 | 2.88 |
| C225 | 16461 | 2.31 |
| hIgG1 | 154 | Negative |

Remark: “Poor fit” indicates that EC50 value could not be calculated.

### Example 6: Detection of Cross-Binding Activity of VHH-Fc Antibodies

6.1 Binding of VHH-Fc Antibodies to EGFR Proteins of Different Species Detected by ELISA

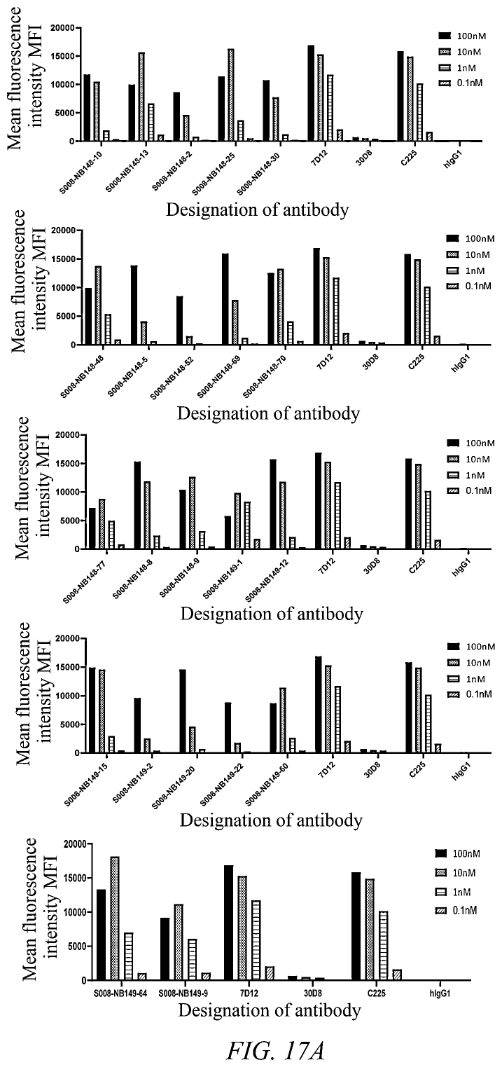

In order to detect the species cross-binding activity of the VHH-Fc antibodies, an ELISA plate was coated with commercial murine EGFR (SB, catalog number: 51091-M08H) and monkey EGFR (SB, catalog number: 90285-C08H), respectively, and the ELISA detection was performed according to the method in Example 4.1. The ELISA results of the VHH-Fc and murine EGFR are shown in FIG. 14 and Table 13, wherein the IgG control is hIgG1; anti-murine EGFR antibody, clone #004 (purchased from SB, catalog number: 51091-R004) is a positive control for the murine EGFR protein. Table 15 shows 22 purified VHH-Fc antibodies, wherein 9 antibodies 5008-NB148-25, 5008-NB148-5, 5008-NB148-69, 5008-NB149-1, S008-NB149-12, 5008-NB149-15, 5008-NB149-20, 5008-NB149-22 and 5008-NB149-60 have no binding to the murine EGFR protein at ELISA level, while other antibodies can bind to the murine EGFR protein at ELISA level.

TABLE 15
Binding reaction of VHH-Fc antibodies with
murine EGFR protein detected by ELISA

| | OD450 | | | | | | | |
|---|---|---|---|---|---|---|---|---|
| | Antibody concentration(nM) | | | | | | | |
| Designation of antibody | 100 | 10 | 1 | 0.1 | 0.01 | 0.001 | 0.0001 | 0 |
| S008-NB148-10 | 2.06 | 0.55 | 0.12 | 0.06 | 0.05 | 0.05 | 0.05 | 0.06 |
| S008-NB148-13 | 3.13 | 2.68 | 2.61 | 2.42 | 1.15 | 0.24 | 0.07 | 0.06 |
| S008-NB148-2 | 2.72 | 2.71 | 2.38 | 1.34 | 0.22 | 0.07 | 0.05 | 0.05 |
| S008-NB148-25 | 0.08 | 0.06 | 0.05 | 0.05 | 0.05 | 0.05 | 0.05 | 0.05 |
| S008-NB148-30 | 2.39 | 2.46 | 1.36 | 0.27 | 0.08 | 0.06 | 0.05 | 0.06 |
| S008-NB148-48 | 2.99 | 2.79 | 2.77 | 2.46 | 0.92 | 0.20 | 0.09 | 0.07 |
| S008-NB148-5 | 0.07 | 0.05 | 0.05 | 0.05 | 0.05 | 0.05 | 0.05 | 0.08 |
| S008-NB148-52 | 2.98 | 2.83 | 2.43 | 1.13 | 0.17 | 0.06 | 0.05 | 0.05 |
| S008-NB148-69 | 0.29 | 0.09 | 0.06 | 0.10 | 0.06 | 0.05 | 0.05 | 0.06 |
| S008-NB148-70 | 3.11 | 2.82 | 2.78 | 2.52 | 1.08 | 0.22 | 0.07 | 0.06 |
| S008-NB148-77 | 2.17 | 2.21 | 2.16 | 1.77 | 0.52 | 0.10 | 0.05 | 0.05 |
| S008-NB148-8 | 2.93 | 2.85 | 2.73 | 2.43 | 1.00 | 0.19 | 0.07 | 0.07 |
| S008-NB148-9 | 3.00 | 2.96 | 2.75 | 2.57 | 1.32 | 0.30 | 0.08 | 0.06 |
| S008-NB149-1 | 0.30 | 0.37 | 0.29 | 0.06 | 0.04 | 0.06 | 0.05 | 0.05 |
| S008-NB149-12 | 0.06 | 0.05 | 0.05 | 0.05 | 0.05 | 0.05 | 0.05 | 0.05 |
| S008-NB149-15 | 0.08 | 0.05 | 0.05 | 0.05 | 0.05 | 0.05 | 0.05 | 0.05 |
| S008-NB149-2 | 2.19 | 1.63 | 0.60 | 0.08 | 0.05 | 0.05 | 0.05 | 0.05 |
| S008-NB149-20 | 0.09 | 0.08 | 0.06 | 0.06 | 0.07 | 0.06 | 0.06 | 0.07 |
| S008-NB149-22 | 0.07 | 0.05 | 0.05 | 0.05 | 0.05 | 0.05 | 0.05 | 0.05 |
| S008-NB149-60 | 0.07 | 0.05 | 0.05 | 0.05 | 0.05 | 0.05 | 0.05 | 0.05 |
| S008-NB149-64 | 1.77 | 1.96 | 1.86 | 1.00 | 0.23 | 0.08 | 0.05 | 0.05 |
| S008-NB149-9 | 1.52 | 1.91 | 0.17 | 0.06 | 0.06 | 0.06 | 0.06 | 0.06 |
| 30D8 | 0.10 | 0.06 | 0.06 | 0.06 | 0.06 | 0.06 | 0.06 | 0.06 |
| 7D12 | 0.30 | 0.10 | 0.06 | 0.06 | 0.06 | 0.06 | 0.05 | 0.09 |
| C225 | 0.18 | 0.13 | 0.07 | 0.07 | 0.06 | 0.06 | 0.06 | 0.06 |
| hIgG1 | 1.16 | 0.06 | 0.12 | 0.06 | 0.06 | 0.05 | 0.05 | 0.06 |

*The dilution factor of the anti-murine EGFR antibody #004 was 1:10000, and the OD450 was 1.72.

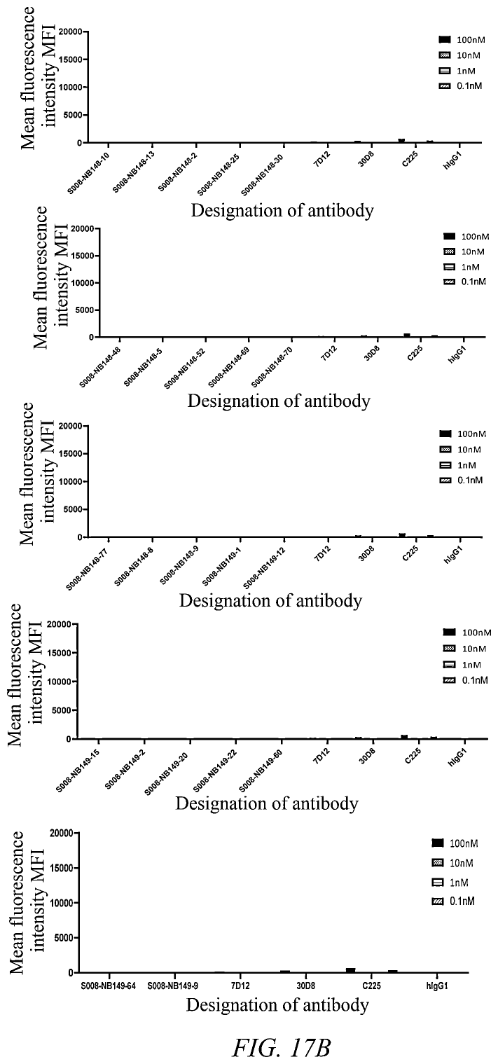

The ELISA results of the VHH-Fc antibodies and the monkey EGFR are shown in FIG. 15 and Table 16, wherein the IgG control is hIgG1; anti-C225 and 7D12 are the positive controls for the monkey EGFR protein. The results showed that S008-NB148-2 had no binding activity to the monkey EGFR protein at ELISA level, while other VHH-Fc antibodies had binding activity to the monkey EGFR protein.

TABLE 16
Binding reaction of VHH-Fc antibodies with
monkey EGFR protein detected by ELISA

| | OD450 | | | | | | | |
|---|---|---|---|---|---|---|---|---|
| | Antibody concentration (nM) | | | | | | | |
| Designation of antibody | 100 | 10 | 1 | 0.1 | 0.01 | 0.001 | 0.0001 | 0 |
| S008-NB148-10 | 2.83 | 2.58 | 2.38 | 2.05 | 0.86 | 0.18 | 0.07 | 0.06 |
| S008-NB148-13 | 2.48 | 2.35 | 2.15 | 2.03 | 0.85 | 0.18 | 0.07 | 0.07 |
| S008-NB148-2 | 0.25 | 0.08 | 0.06 | 0.05 | 0.05 | 0.05 | 0.05 | 0.06 |
| S008-NB148-25 | 2.68 | 2.45 | 2.30 | 2.00 | 0.68 | 0.15 | 0.07 | 0.07 |
| S008-NB148-30 | 2.60 | 2.41 | 2.00 | 0.67 | 0.13 | 0.07 | 0.06 | 0.07 |
| S008-NB148-48 | 2.69 | 2.53 | 2.38 | 1.64 | 0.28 | 0.08 | 0.06 | 0.06 |
| S008-NB148-5 | 2.56 | 2.51 | 2.32 | 1.92 | 0.70 | 0.15 | 0.06 | 0.06 |
| S008-NB148-52 | 2.75 | 2.49 | 2.27 | 2.01 | 0.81 | 0.17 | 0.07 | 0.06 |
| S008-NB148-69 | 2.58 | 2.43 | 2.24 | 1.93 | 0.64 | 0.14 | 0.06 | 0.07 |
| S008-NB148-70 | 2.74 | 2.61 | 2.50 | 2.09 | 0.67 | 0.14 | 0.06 | 0.06 |
| S008-NB148-77 | 2.22 | 2.11 | 1.99 | 1.75 | 0.59 | 0.11 | 0.04 | 0.04 |
| S008-NB148-8 | 2.54 | 2.39 | 2.16 | 1.81 | 0.61 | 0.13 | 0.06 | 0.06 |
| S008-NB148-9 | 2.67 | 2.50 | 2.19 | 1.42 | 0.22 | 0.08 | 0.06 | 0.06 |
| S008-NB149-1 | 1.69 | 1.81 | 1.70 | 1.29 | 0.35 | 0.10 | 0.04 | 0.04 |
| S008-NB149-12 | 2.21 | 2.19 | 2.04 | 1.60 | 0.54 | 0.09 | 0.04 | 0.04 |
| S008-NB149-15 | 2.44 | 2.31 | 2.13 | 1.95 | 0.89 | 0.21 | 0.09 | 0.08 |
| S008-NB149-2 | 2.67 | 2.30 | 2.15 | 1.63 | 0.43 | 0.10 | 0.06 | 0.08 |
| S008-NB149-20 | 2.43 | 2.26 | 2.27 | 1.93 | 0.97 | 0.22 | 0.08 | 0.06 |
| S008-NB149-22 | 2.30 | 2.26 | 1.88 | 1.37 | 0.36 | 0.07 | 0.04 | 0.04 |
| S008-NB149-0 | 2.47 | 2.35 | 2.20 | 1.82 | 0.73 | 0.15 | 0.06 | 0.06 |
| S008-NB149-64 | 2.03 | 2.13 | 1.96 | 1.40 | 0.33 | 0.13 | 0.06 | 0.05 |
| S008-NB149-9 | 2.19 | 2.15 | 1.95 | 1.55 | 0.45 | 0.11 | 0.06 | 0.12 |
| 30D8 | 0.19 | 0.08 | 0.06 | 0.06 | 0.06 | 0.06 | 0.05 | 0.06 |
| 7D12 | 2.34 | 2.26 | 2.14 | 1.87 | 0.63 | 0.13 | 0.06 | 0.06 |
| C225 | 2.26 | 2.31 | 2.16 | 1.90 | 0.68 | 0.14 | 0.06 | 0.12 |
| hIgG1 | 0.71 | 0.13 | 0.21 | 0.09 | 0.19 | 0.09 | 0.06 | 0.07 |

6.2 Binding of VHH-Fc Antibodies to Monkey EGFR Expressing Cells Detected by FACS

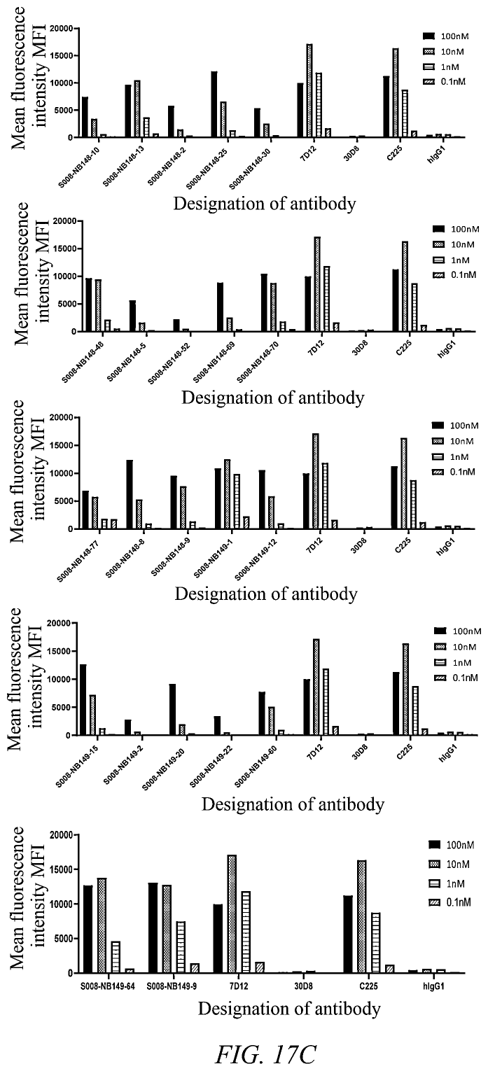

HEK293T-monkey EGFR cells were subjected to FACS detection and data analysis according to the method in Example 4.2. The analysis results are shown in FIG. 16 and Table 17, wherein the IgG control is hIgG1; anti-C225 and 7D12 are the positive controls for the monkey EGFR protein. The results showed that S008-NB148-2 had no binding activity to the monkey EGFR protein on the surface of HEK293T cells, and other VHH-Fc antibodies had binding activity to the monkey EGFR protein on the surface of 293 cells.

TABLE 17
Binding reaction of VHH-Fc antibodies with HEK293T-monkey
EGFR cells detected by FACS

| | Maximum average | |
|---|---|---|
| | fluorescence intensity | EC50 |
| Designation of antibody | Max MFI | nM |
| S008-NB148-10 | 18561 | 11.69 |
| S008-NB148-13 | 18508 | 1.31 |
| S008-NB148-2 | 88 | Negative |
| S008-NB148-25 | 19578 | 3.08 |
| S008-NB148-30 | 2476 | 31.91 |
| S008-NB148-48 | 4749 | 47.61 |
| S008-NB148-5 | 16156 | 27.63 |
| S008-NB148-52 | 9574 | 123.4 |
| S008-NB148-69 | 17575 | 15.87 |
| S008-NB148-70 | 11983 | 2.13 |
| S008-NB148-77 | 12653 | 2.18 |
| S008-NB148-8 | 12922 | 2.18 |
| S008-NB148-9 | 2901 | 20.12 |
| S008-NB149-1 | 16491 | 1.10 |
| S008-NB149-12 | 18880 | 8.87 |
| S008-NB149-15 | 19163 | 3.89 |
| S008-NB149-2 | 1373 | 174.4 |
| S008-NB149-20 | 16979 | 27.95 |
| S008-NB149-22 | 9273 | Poor fit |
| S008-NB149-60 | 14118 | 2.68 |
| S008-NB149-64 | 18730 | 1.17 |
| S008-NB149-9 | 8957 | 0.57 |
| 30D8 | 77 | Negative |
| 7D12 | 18711 | 0.70 |
| C225 | 21057 | ~0.85 |
| hIgG1 | 240 | Negative |

Remark: “Poor fit” indicates that EC50 value could not be calculated.

### Example 7: EGFR Antibody Specificity Assay

7.1 Assay of Specificity of VHH-Fc to Human EGFR Protein

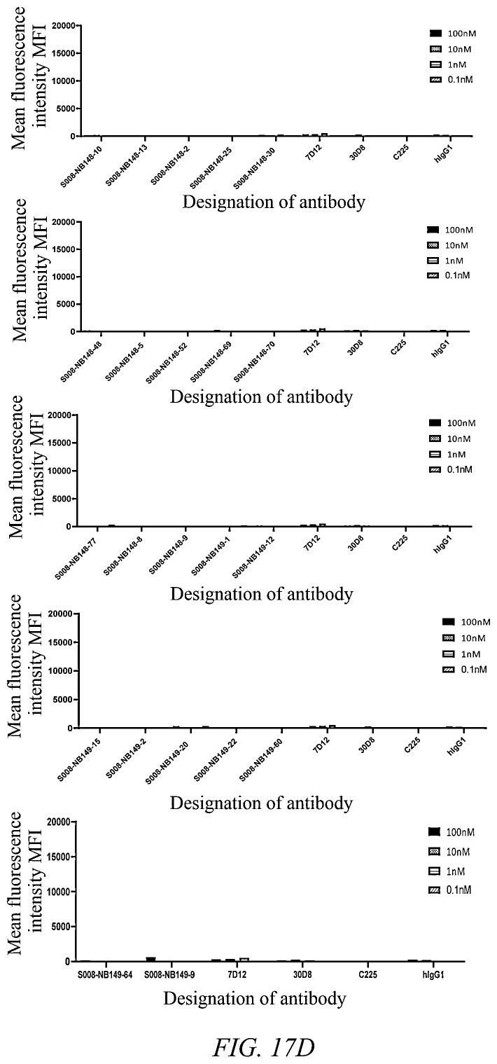

The endogenous cell A431 expressing human EGFR, the transfected cell line CHO-K1-human EGFR 1D4 cells, the cell line MCF-7 without human EGFR expression and the CHO-K1 blank cells were subjected to FACS detection and data analysis according to the method in Example 4.2. The analysis results are shown in Table 18 and FIGS. 17A-17D, wherein the IgG control is hIgG1; anti-C225 and 7D12 are the positive controls for the monkey EGFR protein. The results showed that all VHH-Fc antibodies had specific binding activity to the human EGFR protein on the cell surface.

TABLE 18
Binding reaction of VHH-Fc antibodies with human
EGFR expressing cells detected by FACS

| | Maximum average fluorescence | | | |
|---|---|---|---|---|
| | intensity/Max_MFI | | | |
| Designation of | | | CHO-K1-human | CHO-K1 |
| antibody | A431 | MCF-7 | EGFR 1D4 | Blank cell |
| S008-NB148-10 | 11783 | 83 | 7444 | 229 |
| S008-NB148-13 | 15675 | 118 | 10490 | 101 |
| S008-NB148-2 | 8640 | 77 | 5801 | 89 |
| S008-NB148-25 | 16314 | 135 | 12129 | 141 |
| S008-NB148-30 | 10761 | 173 | 5381 | 322 |
| S008-NB148-48 | 13781 | 114 | 9670 | 175 |
| S008-NB148-5 | 13869 | 96 | 5652 | 151 |
| S008-NB148-52 | 8485 | 71 | 2245 | 135 |
| S008-NB148-69 | 15965 | 91 | 8860 | 254 |
| S008-NB148-70 | 13267 | 79 | 10460 | 98 |
| S008-NB148-77 | 8817 | 79 | 6879 | 365 |
| S008-NB148-8 | 15340 | 94 | 12401 | 160 |
| S008-NB148-9 | 12658 | 125 | 9562 | 86 |
| S008-NB149-1 | 9851 | 168 | 12531 | 227 |
| S008-NB149-12 | 15735 | 82 | 10578 | 184 |
| S008-NB149-15 | 14938 | 82 | 12623 | 93 |
| S008-NB149-2 | 9632 | 79 | 2816 | 91 |
| S008-NB149-20 | 14597 | 103 | 9158 | 363 |
| S008-NB149-22 | 8848 | 91 | 3422 | 189 |
| S008-NB149-60 | 11418 | 76 | 7748 | 153 |
| S008-NB149-64 | 18177 | 128 | 13820 | 171 |
| S008-NB149-9 | 11211 | 80 | 13107 | 631 |
| 30D8 | 712 | 343 | 298 | 369 |
| 7D12 | 16914 | 205 | 17157 | 561 |
| C225 | 15855 | 714 | 16356 | 164 |
| hIgG1 | 199 | 87 | 662 | 290 |

7.2 Assay of Specificity of VHH-Fc to Monkey EGFR Protein

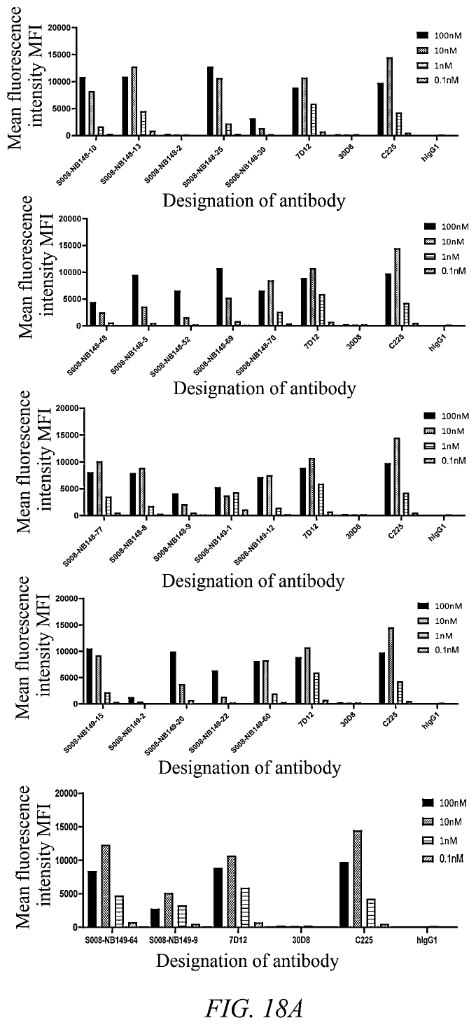

The transfected cell line HEK293T-monkey EGFR cells with the monkey EGFR expression and the cell line 293 blank cells without the monkey EGFR expression were subjected to FACS detection and data analysis according to the method in Example 4.2. The analysis results are shown in Table 19 and FIGS. 18A-18B, wherein the IgG control is hIgG1; anti-C225 and 7D12 are the positive controls for the monkey EGFR protein. The results showed that 5008-NB148-2 had no binding activity to the monkey EGFR protein on the surface of HEK293T cells, and other VHH-Fc antibodies had specific binding activity to the monkey EGFR protein on the surface of HEK293T cells.

TABLE 19
Binding reaction of VHH-Fc antibodies with monkey
EGFR expressing cells detected by FACS

| | Maximum average fluorescence | |
|---|---|---|
| | intensity/Max_MFI | |
| Designation | HEK293T- | HEK293T |
| of antibody | monkey EGFR | Blank cell |
| S008-NB148-10 | 10851 | 312 |
| S008-NB148-13 | 12809 | 303 |
| S008-NB148-2 | 359 | 99 |
| S008-NB148-25 | 12788 | 356 |
| S008-NB148-30 | 3222 | 161 |
| S008-NB148-48 | 4455 | 138 |
| S008-NB148-5 | 9547 | 245 |
| S008-NB148-52 | 6616 | 158 |
| S008-NB148-69 | 10752 | 304 |
| S008-NB148-70 | 8505 | 109 |
| S008-NB148-77 | 10126 | 84 |
| S008-NB148-8 | 8909 | 310 |
| S008-NB148-9 | 4169 | 101 |
| S008-NB149-1 | 5297 | 490 |
| S008-NB149-12 | 7545 | 387 |
| S008-NB149-15 | 10519 | 240 |
| S008-NB149-2 | 1326 | 91 |
| S008-NB149-20 | 9965 | 311 |
| S008-NB149-22 | 6371 | 161 |
| S008-NB149-60 | 8306 | 96 |
| S008-NB149-64 | 12375 | 400 |
| S008-NB149-9 | 5169 | 464 |
| 30D8 | 314 | 134 |
| 7D12 | 10751 | 336 |
| C225 | 14520 | 545 |
| hIgG1 | 141 | 93 |

7.3 Assay of Specificity of VHH-Fc to Human EGFR Family Protein

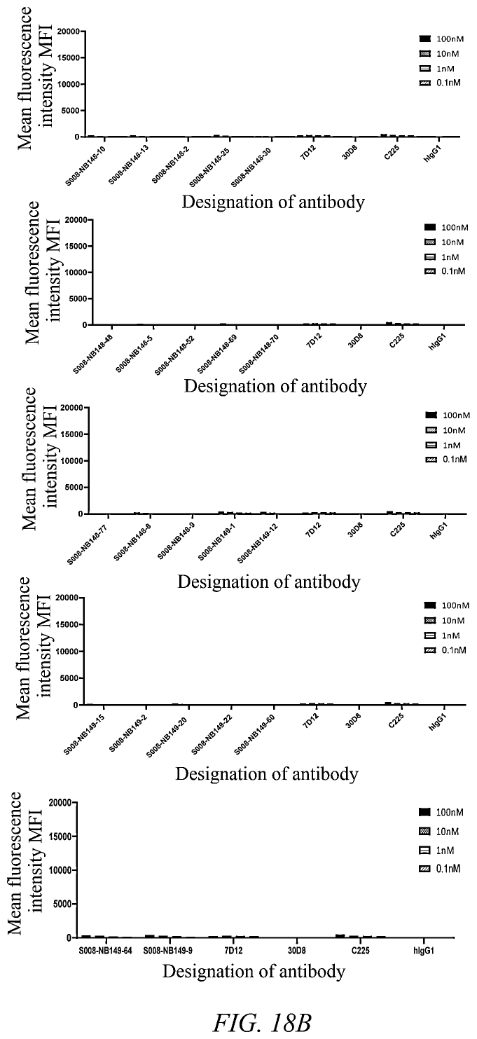

In order to detect the binding specificity of the VHH-Fc antibodies to the EGFR family protein, an ELISA plate was coated with the commercial Her2 protein (purchased from Acro, catalog number: HE2-H5225), and ELISA detection was performed according to the method in Example 4.1. The ELISA results of the VHH-Fc and the Her2 protein are shown in FIG. 19 and Table 20, wherein the IgG control is hIgG1; 4D5 is the positive control for the Her2 protein. Only one concentration of 10 nM was set for the detection. The detection result OD450 was 4.00, which was more than 10 times different from that of hIgG1 at the same concentration, indicating that this experiment can be used to determine the binding reaction of the VHH-Fc antibodies with the EGFR family protein Her2. The results showed that all the purified VHH-Fc antibodies had no binding to the human Her2 protein at ELISA level.

TABLE 20
Binding reaction of VHH-Fc antibodies with Her2 detected by ELISA

| | OD450 | | | | | | | |
|---|---|---|---|---|---|---|---|---|
| Designation of | Antibody concentration(nM) | | | | | | | |
| antibody | 100 | 10 | 1 | 0.1 | 0.01 | 0.001 | 0.0001 | 0 |
| S008-NB148-10 | 0.25 | 0.13 | 0.13 | 0.09 | 0.11 | 0.08 | 0.15 | 0.10 |
| S008-NB148-13 | 0.14 | 0.20 | 0.15 | 0.13 | 0.13 | 0.13 | 0.15 | 0.21 |
| S008-NB148-2 | 0.13 | 0.17 | 0.10 | 0.11 | 0.07 | 0.07 | 0.11 | 0.12 |
| S008-NB148-25 | 0.24 | 0.11 | 0.11 | 0.11 | 0.14 | 0.16 | 0.10 | 0.11 |
| S008-NB148-30 | 0.17 | 0.12 | 0.10 | 0.09 | 0.12 | 0.12 | 0.08 | 0.15 |
| S008-NB148-48 | 0.16 | 0.11 | 0.11 | 0.06 | 0.06 | 0.08 | 0.11 | 0.16 |
| S008-NB148-5 | 0.18 | 0.11 | 0.18 | 0.10 | 0.09 | 0.32 | 0.11 | 0.12 |
| S008-NB148-52 | 0.17 | 0.28 | 0.12 | 0.15 | 0.12 | 0.10 | 0.11 | 0.21 |
| S008-NB148-69 | 0.18 | 0.09 | 0.11 | 0.11 | 0.11 | 0.12 | 0.08 | 0.14 |
| S008-NB148-70 | 0.16 | 0.14 | 0.08 | 0.09 | 0.15 | 0.10 | 0.10 | 0.16 |
| S008-NB148-77 | 0.18 | 0.13 | 0.12 | 0.12 | 0.11 | 0.12 | 0.12 | 0.11 |
| S008-NB148-8 | 0.12 | 0.12 | 0.10 | 0.10 | 0.08 | 0.09 | 0.10 | 0.11 |
| S008-NB148-9 | 0.15 | 0.12 | 0.12 | 0.11 | 0.10 | 0.08 | 0.09 | 0.14 |
| S008-NB149-1 | 0.15 | 0.13 | 0.09 | 0.09 | 0.07 | 0.13 | 0.16 | 0.23 |
| S008-NB149-12 | 0.17 | 0.14 | 0.11 | 0.13 | 0.09 | 0.15 | 0.15 | 0.10 |
| S008-NB149-15 | 0.18 | 0.17 | 0.13 | 0.16 | 0.20 | 0.16 | 0.13 | 0.15 |
| S008-NB149-2 | 0.21 | 0.12 | 0.08 | 0.19 | 0.14 | 0.14 | 0.22 | 0.18 |
| S008-NB149-20 | 0.18 | 0.18 | 0.13 | 0.12 | 0.14 | 0.16 | 0.17 | 0.14 |
| S008-NB149-22 | 0.17 | 0.12 | 0.08 | 0.08 | 0.11 | 0.11 | 0.12 | 0.16 |
| S008-NB149-60 | 0.23 | 0.16 | 0.10 | 0.19 | 0.15 | 0.14 | 0.17 | 0.15 |
| S008-NB149-64 | 0.17 | 0.09 | 0.07 | 0.07 | 0.15 | 0.08 | 0.13 | 0.17 |
| S008-NB149-9 | 0.16 | 0.12 | 0.05 | 0.09 | 0.09 | 0.11 | 0.09 | 0.18 |
| 7D12 | 0.14 | 0.10 | 0.10 | 0.09 | 0.08 | 0.10 | 0.11 | 0.13 |
| C225 | 0.25 | 0.17 | 0.08 | 0.11 | 0.10 | 0.10 | 0.11 | 0.15 |
| 4D5 | Not | 4.00 | Not | Not | Not | Not | Not | Not |
| | determined | | determined | determined | determined | determined | determined | determined |
| hIgG1 | 0.21 | 0.11 | 0.12 | 0.14 | 0.11 | 0.12 | 0.11 | 0.13 |

### Example 8: EGFR Antibody Affinity Assay

8.1 Assay of Affinity of VHH-Fc to Human EGFR Protein

Anti-human EGFR VHH-Fc antibodies were captured using a Protein A chip (GE Helthcare; 29-127-558). Sample buffer and running buffer were HBS-EP+ (10 mM HEPES, 150 mM NaCl, 3 mM EDTA, 0.05% surfactant P20) (GE Healthcare; BR-1006-69). The flow-through cell was set to 25° C. The sample block was set to 16° C. Both were pretreated with the running buffer. In each cycle, the antibody to be tested was first captured with a Protein A chip, then a single concentration of EGFR antigen protein was injected. The binding and dissociation process of the antibody and the antigen protein was recorded, and finally Glycine pH 1.5 (GE Helthcare; BR-1003-54) was used to complete chip regeneration. Binding was measured by injecting different concentrations of recombinant human EGFR in solution for 240 s with a flow rate of 30 μL/min. The concentration started from 200 nM (see the detailed results for the actual concentration in the test) and was diluted at 1:1, making a total of 5 concentrations. The dissociation phase was monitored for up to 600 s and was triggered by switching from sample solution to running buffer. The surface was regenerated by washing with a 10 mM of glycine solution (pH 1.5) for 30 s at a flow rate of 30 μL/min. Bulk refractive index difference was corrected by subtracting the response obtained from the goat anti-human Fc surface, and the blank injection was subtracted at the same time (=double reference). For calculation of apparent KD value and other kinetic parameters, Langmuir 1:1 model was used. The binding rate (Ka), dissociation rate (Kd) and binding affinity (KD) of the VHH-Fc antibodies to human EGFR protein are shown in Table 21, wherein the antibodies C225 and 7D12 were used as positive controls. As shown in Table 21, all the VHH-Fc antibodies bind to the human EGFR protein with an affinity better than 5.21E-07M.

TABLE 21
Affinity of VHH-Fc antibodies to human EGFR
detected by SPR (biacore)

| Designation of | Binding kinetics | | |
|---|---|---|---|
| antibody | ka(1/Ms) | kd(1/s) | KD (M) |
| S008-NB148-10 | 4.24E+04 | 2.97E−03 | 7.00E−08 |
| S008-NB148-13 | 4.24E+04 | 1.09E−02 | 2.57E−07 |
| S008-NB148-2 | 9.98E+03 | 5.20E−03 | 5.21E−07 |
| S008-NB148-25 | 1.92E+04 | 1.41E−03 | 7.31E−08 |
| S008-NB148-30 | 5.86E+04 | 3.17E−04 | 5.41E−09 |
| S008-NB148-48 | 5.88E+08 | 9.55E+00 | 1.63E−08 |
| S008-NB148-5 | 3.33E+04 | 3.25E−04 | 9.75E−09 |
| S008-NB148-52 | 2.78E+03 | 5.79E−04 | 2.08E−07 |
| S008-NB148-69 | 3.64E+04 | 3.69E−04 | 1.01E−08 |
| S008-NB148-70 | 1.64E+05 | 1.18E−02 | 7.18E−08 |
| S008-NB148-77 | 9.18E+08 | 1.80E+01 | 1.96E−08 |
| S008-NB148-8 | 4.97E+04 | 3.28E−03 | 6.61E−08 |
| S008-NB148-9 | 2.99E+04 | 1.41E−02 | 4.72E−07 |
| S008-NB149-1 | 1.12E+05 | 6.59E−03 | 5.87E−08 |
| S008-NB149-12 | 4.37E+04 | 1.70E−03 | 3.90E−08 |
| S008-NB149-15 | 1.51E+05 | 6.95E−03 | 4.59E−08 |
| S008-NB149-2 | 6.47E+04 | 1.98E−03 | 3.07E−08 |
| S008-NB149-20 | 9.41E+04 | 1.46E−03 | 1.55E−08 |
| S008-NB149-22 | 8.49E+03 | 1.02E−03 | 1.20E−07 |
| S008-NB149-60 | 7.51E+04 | 1.97E−03 | 2.62E−08 |
| S008-NB149-64 | 9.09E+04 | 6.80E−05 | 7.48E−10 |
| S008-NB149-9 | 2.35E+05 | 8.62E−03 | 3.66E−08 |
| 30D8 | Negative | Negative | Negative |
| 7D12 | 3.33E+05 | 1.33E−03 | 4.00E−09 |
| C225 | 1.40E+06 | 4.63E−04 | 3.30E−10 |

8.2 Assay of Affinity of VHH-Fc to Human EGFRvIII Protein

The affinity of the VHH-Fc antibodies to human EGFRvIII protein was determined according to the method of Example 8.1, wherein the antibodies 7D12, C225, and 30D8 were used as positive controls. As shown in Table 22, no binding signal was detected between the VHH-Fc antibodies S008-NB148-2 and S008-NB149-64 and the human EGFRvIII, while other antibodies binded to the human EGFRvIII protein with an affinity better than 5.64E-08M.

TABLE 22
Affinity of VHH-Fc antibodies to human EGFRvIII
detected by SPR (biacore)

| Designation of | Binding kinetics | | |
|---|---|---|---|
| antibody | ka(1/Ms) | kd(1/s) | KD (M) |
| S008-NB148-10 | 6.10E+04 | 1.79E−03 | 2.93E−08 |
| S008-NB148-13 | 4.86E+04 | 2.46E−03 | 5.05E−08 |
| S008-NB148-2 | Negative | Negative | Negative |
| S008-NB148-25 | 4.24E+04 | 7.27E−04 | 1.72E−08 |
| S008-NB148-30 | 2.86E+08 | 3.46E+00 | 1.21E−08 |
| S008-NB148-48 | 2.85E+08 | 6.66E+00 | 2.34E−08 |
| S008-NB148-5 | 3.18E+04 | 9.47E−05 | 2.98E−09 |
| S008-NB148-52 | 5.87E+04 | 4.38E−04 | 7.46E−09 |
| S008-NB148-69 | 6.31E+04 | 1.65E−04 | 2.62E−09 |
| S008-NB148-70 | 1.36E+05 | 5.54E−03 | 4.08E−08 |
| S008-NB148-77 | 1.90E+05 | 1.88E−03 | 9.91E−09 |
| S008-NB148-8 | 7.66E+04 | 3.25E−03 | 4.25E−08 |
| S008-NB148-9 | 5.76E+07 | 3.25E+00 | 5.64E−08 |
| S008-NB149-1 | 7.41E+08 | 1.72E+01 | 2.32E−08 |
| S008-NB149-12 | 6.02E+04 | 4.75E−04 | 7.89E−09 |
| S008-NB149-15 | 4.27E+05 | 1.67E−02 | 3.91E−08 |
| S008-NB149-2 | 1.68E+05 | 3.44E−03 | 2.05E−08 |
| S008-NB149-20 | 4.52E+04 | 3.94E−04 | 8.70E−09 |
| S008-NB149-22 | 4.64E+04 | 1.42E−03 | 3.07E−08 |
| S008-NB149-60 | 1.61E+05 | 2.10E−03 | 1.30E−08 |
| S008-NB149-64 | Negative | Negative | Negative |
| S008-NB149-9 | 5.32E+08 | 1.33E+01 | 2.49E−08 |
| 30D8 | 1.97E+05 | 2.29E−03 | 1.17E−08 |
| 7D12 | 3.40E+05 | 1.27E−03 | 3.73E−09 |
| C225 | 1.87E+06 | 5.74E−04 | 3.06E−10 |

8.3 Assay of Affinity of VHH-Fc to Monkey EGFR Protein

The affinity of the VHH-Fc antibodies to the monkey EGFR protein described above was determined according to the method of Example 8.1, wherein the antibodies 7D12 and C225 were used as positive controls, and 30D8 was used as a negative control. As shown in Table 23, no binding signal was detected between the S008-NB148-2 antibody and the monkey EGFR protein, and the other antibodies all binded to the monkey EGFR protein with an affinity better than 2.44E-08M.

TABLE 23
Affinity of VHH-Fc antibodies to monkey EGFR
detected by SPR (biacore)

| | Binding kinetics | | |
|---|---|---|---|
| Antibody | ka (1/Ms) | kd (1/s) | KD (M) |
| S008-NB148-10 | 6.04E+04 | 6.39E−04 | 1.06E−08 |
| S008-NB148-13 | 5.09E+04 | 6.16E−04 | 1.21E−08 |
| S008-NB148-2 | Negative | Negative | Negative |
| S008-NB148-25 | 2.39E+04 | 5.83E−04 | 2.44E−08 |
| S008-NB148-30 | 7.17E+04 | 1.85E−04 | 2.58E-09 |
| S008-NB148-48 | 9.54E+04 | 5.23E-10 | 1.00E-09 |
| S008-NB148-5 | 4.87E+04 | 1.58E−04 | 3.25E−09 |
| S008-NB148-52 | 5.96E+04 | 3.95E−04 | 6.63E−09 |
| S008-NB148-69 | 5.36E+04 | 1.76E-04 | 3.28E−09 |
| S008-NB148-70 | 7.30E+04 | 1.54E−04 | 2.11E−09 |
| S008-NB148-77 | 2.56E+05 | 4.46E−04 | 1.74E−09 |
| S008-NB148-8 | 6.63E+04 | 1.72E−04 | 2.59E-09 |
| S008-NB148-9 | 8.74E+04 | 6.59E−04 | 7.54E−09 |
| S008-NB149-1 | 1.19E+05 | 6.49E−04 | 5.46E−09 |
| S008-NB149-12 | 5.56E+04 | 4.61E−04 | 8.30E−09 |
| S008-NB149-15 | 8.18E+04 | 6.79E−04 | 8.30E-09 |
| S008-NB149-2 | 6.06E+04 | 4.82E−04 | 7.95E−09 |
| S008-NB149-20 | 3.86E+04 | 1.02E−04 | 2.63E−09 |
| S008-NB149-22 | 7.17E+04 | 6.85E−04 | 9.55E−09 |
| S008-NB149-60 | 8.82E+04 | 1.48E−04 | 1.68E−09 |
| S008-NB149-64 | 1.09E+05 | 6.43E−05 | 5.87E−10 |
| S008-NB149-9 | 1.09E+05 | 1.47E−03 | 1.35E−08 |
| 30D8 | Negative | Negative | Negative |
| 7D12 | 4.13E+05 | 1.93E−03 | 4.66E−09 |
| C225 | 1.46E+06 | 3.75E-04 | 2.56E−10 |

8.4 Assay of Affinity of VHH-Fc to Murine EGFR Protein

The affinity of the VHH-Fc antibodies to the murine EGFR protein described above was determined according to the method of Example 8.1. As shown in Table 24, the antibodies S008-NB148-77, S008-NB148-8, and S008-NB149-64 binded to the murine EGFR with an affinity better than 5.30E-08M, and no binding signal was detected for the other antibodies.

TABLE 24
Affinity of VHH-Fc antibodies to murine EGFR
detected by SPR (biacore)

| | Binding kinetics | | |
|---|---|---|---|
| Antibody | ka(1/Ms) | kd(1/s) | KD(M) |
| S008-NB148-10 | Negative | Negative | Negative |
| S008-NB148-13 | Negative | Negative | Negative |
| S008-NB148-2 | Negative | Negative | Negative |
| S008-NB148-25 | Negative | Negative | Negative |
| S008-NB148-30 | Negative | Negative | Negative |
| S008-NB148-48 | Negative | Negative | Negative |
| S008-NB148-5 | Negative | Negative | Negative |
| S008-NB148-52 | Negative | Negative | Negative |
| S008-NB148-69 | Negative | Negative | Negative |
| S008-NB148-70 | Negative | Negative | Negative |
| S008-NB148-77 | 2.90E+08 | 3.50E+00 | 1.21E−08 |
| S008-NB148-8 | 3.23E+05 | 1.71E−02 | 5.30E−08 |
| S008-NB148-9 | Negative | Negative | Negative |
| S008-NB149-1 | Negative | Negative | Negative |
| S008-NB149-12 | Negative | Negative | Negative |
| S008-NB149-15 | Negative | Negative | Negative |
| S008-NB149-2 | Negative | Negative | Negative |
| S008-NB149-20 | Negative | Negative | Negative |
| S008-NB149-22 | Negative | Negative | Negative |
| S008-NB149-60 | Negative | Negative | Negative |
| S008-NB149-64 | 1.05E+05 | 1.01E−04 | 9.59E−10 |
| S008-NB149-9 | Negative | Negative | Negative |
| 30D8 | Negative | Negative | Negative |
| 7D12 | Negative | Negative | Negative |
| C225 | Negative | Negative | Negative |

### Example 9 Antibody Antigen-Binding Epitope Analysis

9.1 Identification of Antigen-Binding Region of Antibody

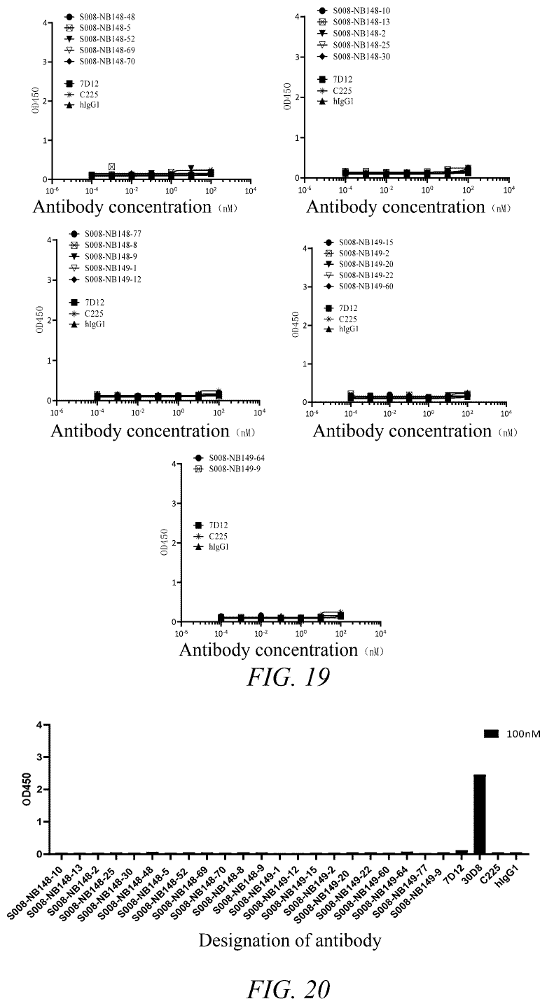

In order to identify the antigen-binding epitope distribution of the VHH antibodies against EGFR, the pepvIII polypeptide obtained in Example 2.4 was used for coating according to the ELISA method in Example 4.1. As shown in FIG. 20, in which the antibody 30D8 was used as a positive control, all the VHH-Fc antibodies did not bind to the polypeptide pepvIII. Except for S008-NB149-64 which did not bind to the EGFRvIII protein, the binding sites of other antibodies were not on the N-terminal polypeptide pepvIII of the EGFRvIII protein.

9.2 Antibody Antigen-Binding Epitope Competition Experiment (Epitope Binning)

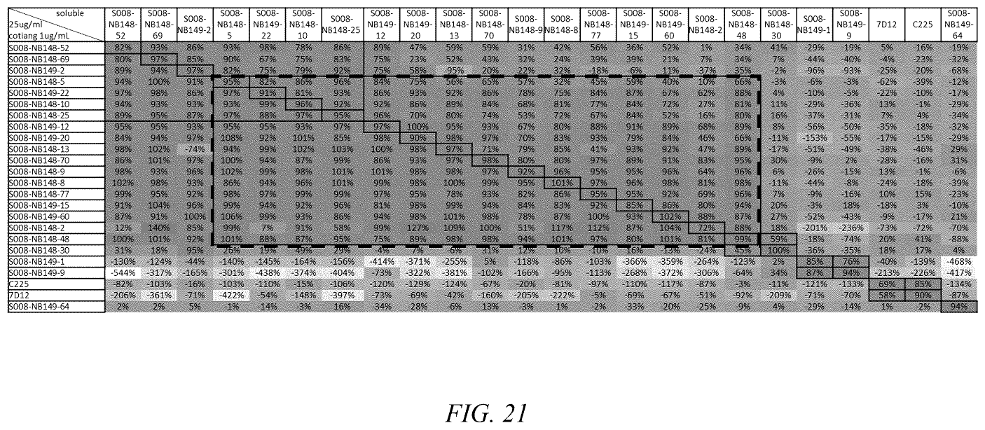

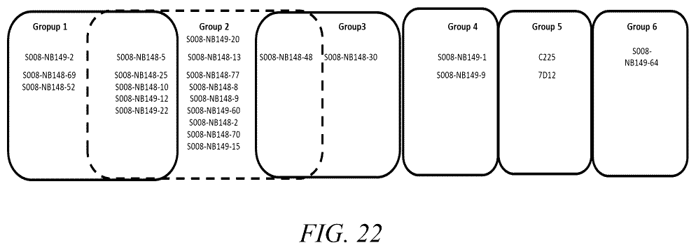

Competitive ELISA was used to classify the epitopes of VHH antibodies and control antibody with known epitopes. According to the method in Example 4.2, the ELISA plate was coated with 1 μg/mL of the antibody, and the human EGFR protein was serially diluted starting from 30 μg/mL, and the EC80 value was calculated (Table 25). The ELISA plate was coated with 1 μg/mL of the antibody, 25 μg/mL of the antibody to be detected was added, and then human EGFR protein corresponding to each antibody coated on the plate was added at the EC80 concentration, incubated for 2 hours, and washed 5 times with PBS, and then an HRP-labeled anti-His antibody (purchased from GenScrip, catalog number: A00612) was added for detection. If there was no competitive relationship between the antibody coated on the plate and the antibody to be detected in the solution, then the antibody coated on the plate could bind to the complex of the antibody to be detected and the human EGFR antigen in solution, and the absorption at OD450 nm was detected, and the inhibition rate between each pair of antibodies was calculated according to the absorbance at OD450 nm (FIG. 21). According to the inhibition rate, each antibody epitope was classified as shown in FIG. 22. The 24 antibodies including positive antibodies can be divided into 6 groups. S008-NB149-2, 5008-NB148-69 and 5008-NB148-52 were classified as the first group; 5008-NB149-20, 5008-NB148-13, 5008-NB148-77, 5008-NB148-8, 5008-NB148-9, S008-NB149-60, S008-NB148-2, S008-NB148-70 and S008-NB149-15 were classified as the second group; 5008-NB148-5, 5008-NB148-25, 5008-NB148-10, 5008-NB149-12 and S008-NB149-22 antibodies competed with both the first group of antibodies and the second group of antibodies; 5008-NB148-30 was classified as the third group; 5008-NB148-48 competed with both the second group of antibodies and the third group of antibodies; 5008-NB149-1 and 5008-NB149-9 was classified as the fourth group; C225 and 7D12 was classified as the fifth group, all the antibodies had no competitive relationship with the positive antibodies C225 and 7D12; and S008-NB149-64 was classified as the sixth group.

TABLE 25
EC80 value of human EGFR protein corresponding
to VHH antibody

| EGFR | EC80 μg/mL |
|---|---|
| hIgG1 | Negative |
| S008-NB148-10 | 10.5 |
| S008-NB148-13 | 1.1 |
| S008-NB148-2 | 15.2 |
| S008-NB148-25 | 8.3 |
| S008-NB148-30 | 21.1 |
| S008-NB148-48 | 64.2 |
| S008-NB148-5 | 20.1 |
| S008-NB148-52 | 2.9 |
| S008-NB148-69 | 3.3 |
| S008-NB148-70 | 37.4 |
| S008-NB148-77 | 46.2 |
| S008-NB148-8 | 76.0 |
| S008-NB148-9 | 7.9 |
| S008-NB149-1 | 3.0 |
| S008-NB149-12 | 2.2 |
| S008-NB149-15 | 2.4 |
| S008-NB149-2 | 2.9 |
| S008-NB149-20 | 2.6 |
| S008-NB149-22 | 4.6 |
| S008-NB149-60 | 5.4 |
| S008-NB149-64 | 0.1 |
| S008-NB149-9 | 24.5 |
| 30D8 | Negative |
| 7D12 | 138.9 |
| C225 | 862 |

1. A nanobody or an antigen-binding fragment that specifically binds to EGFR and EGFRvIII, wherein the nanobody or the antigen-binding fragment comprises a combination of CDRs, the combination of CDRs comprises: CDR1, CDR2, and CDR3; the CDR1, CDR2 and CDR3 have any sequence combination selected from the following or a sequence combination with 1, 2, 3 or more amino acid insertions, deletions and/or substitutions compared to the sequence combination:

| | SEQ ID NO. | | |
|---|---|---|---|
| No. | CDR1 | CDR2 | CDR3 |
| VH1 | SEQ ID NO. 63 | SEQ ID NO. 64 | SEQ ID NO. 65 |
| VH2 | SEQ ID NO. 66 | SEQ ID NO. 67 | SEQ ID NO. 68 |
| VH3 | SEQ ID NO. 69 | SEQ ID NO. 70 | SEQ ID NO. 71 |
| VH4 | SEQ ID NO. 72 | SEQ ID NO. 73 | SEQ ID NO. 74 |
| VH5 | SEQ ID NO. 75 | SEQ ID NO. 76 | SEQ ID NO. 77 |
| VH6 | SEQ ID NO. 78 | SEQ ID NO. 79 | SEQ ID NO. 80 |
| VH7 | SEQ ID NO. 81 | SEQ ID NO. 82 | SEQ ID NO. 83 |
| VH8 | SEQ ID NO. 84 | SEQ ID NO. 85 | SEQ ID NO. 86 |
| VH9 | SEQ ID NO. 87 | SEQ ID NO. 88 | SEQ ID NO. 89 |
| VH10 | SEQ ID NO. 90 | SEQ ID NO. 91 | SEQ ID NO. 92 |
| VH11 | SEQ ID NO. 93 | SEQ ID NO. 94 | SEQ ID NO. 95 |
| VH12 | SEQ ID NO. 96 | SEQ ID NO. 97 | SEQ ID NO. 98 |
| VH13 | SEQ ID NO. 99 | SEQ ID NO. 100 | SEQ ID NO. 101 |
| VH14 | SEQ ID NO. 102 | SEQ ID NO. 103 | SEQ ID NO. 104 |
| VH15 | SEQ ID NO. 105 | SEQ ID NO. 106 | SEQ ID NO. 107 |
| VH16 | SEQ ID NO. 108 | SEQ ID NO. 109 | SEQ ID NO. 110 |
| VH17 | SEQ ID NO. 111 | SEQ ID NO. 112 | SEQ ID NO. 113 |
| VH18 | SEQ ID NO. 114 | SEQ ID NO. 115 | SEQ ID NO. 116 |
| VH19 | SEQ ID NO. 117 | SEQ ID NO. 118 | SEQ ID NO. 119 |
| VH20 | SEQ ID NO. 120 | SEQ ID NO. 121 | SEQ ID NO. 122 |
| VH21 | SEQ ID NO. 123 | SEQ ID NO. 124 | SEQ ID NO. 125 |
| VH22 | SEQ ID NO. 126 | SEQ ID NO. 127 | SEQ ID NO. 128 |
| VH23 | SEQ ID NO. 129 | SEQ ID NO. 130 | SEQ ID NO. 131 |
| VH24 | SEQ ID NO. 132 | SEQ ID NO. 133 | SEQ ID NO. 134 |
| VH25 | SEQ ID NO. 135 | SEQ ID NO. 136 | SEQ ID NO. 137 |
| VH26 | SEQ ID NO. 138 | SEQ ID NO. 139 | SEQ ID NO. 140 |
| VH27 | SEQ ID NO. 141 | SEQ ID NO. 142 | SEQ ID NO. 143 |
| VH28 | SEQ ID NO. 144 | SEQ ID NO. 145 | SEQ ID NO. 146 |
| VH29 | SEQ ID NO. 147 | SEQ ID NO. 148 | SEQ ID NO. 149 |
| VH30 | SEQ ID NO. 150 | SEQ ID NO. 151 | SEQ ID NO. 152 |
| VH31 | SEQ ID NO. 153 | SEQ ID NO. 154 | SEQ ID NO. 155 |
| VH32 | SEQ ID NO. 156 | SEQ ID NO. 157 | SEQ ID NO. 158 |
| VH33 | SEQ ID NO. 159 | SEQ ID NO. 160 | SEQ ID NO. 161 |
| VH34 | SEQ ID NO. 162 | SEQ ID NO. 163 | SEQ ID NO. 164 |
| VH35 | SEQ ID NO. 165 | SEQ ID NO. 166 | SEQ ID NO. 167 |
| VH36 | SEQ ID NO. 168 | SEQ ID NO. 169 | SEQ ID NO. 170 |
| VH37 | SEQ ID NO. 171 | SEQ ID NO. 172 | SEQ ID NO. 173 |
| VH38 | SEQ ID NO. 174 | SEQ ID NO. 175 | SEQ ID NO. 176 |
| VH39 | SEQ ID NO. 177 | SEQ ID NO. 178 | SEQ ID NO. 179 |
| VH40 | SEQ ID NO. 180 | SEQ ID NO. 181 | SEQ ID NO. 182 |
| VH41 | SEQ ID NO. 183 | SEQ ID NO. 184 | SEQ ID NO. 185 |
| VH42 | SEQ ID NO. 186 | SEQ ID NO. 187 | SEQ ID NO. 188 |
| VH43 | SEQ ID NO. 189 | SEQ ID NO. 190 | SEQ ID NO. 191 |
| VH44 | SEQ ID NO. 192 | SEQ ID NO. 193 | SEQ ID NO. 194 |
| VH45 | SEQ ID NO. 195 | SEQ ID NO. 196 | SEQ ID NO. 197 |
| VH46 | SEQ ID NO. 198 | SEQ ID NO. 199 | SEQ ID NO. 200 |
| VH47 | SEQ ID NO. 201 | SEQ ID NO. 202 | SEQ ID NO. 203 |
| VH48 | SEQ ID NO. 204 | SEQ ID NO. 205 | SEQ ID NO. 206 |
| VH49 | SEQ ID NO. 207 | SEQ ID NO. 208 | SEQ ID NO. 209 |
| VH50 | SEQ ID NO. 210 | SEQ ID NO. 211 | SEQ ID NO. 212 |
| VH51 | SEQ ID NO. 213 | SEQ ID NO. 214 | SEQ ID NO. 215 |
| VH52 | SEQ ID NO. 216 | SEQ ID NO. 217 | SEQ ID NO. 218 |
| VH53 | SEQ ID NO. 219 | SEQ ID NO. 220 | SEQ ID NO. 221 |
| VH54 | SEQ ID NO. 222 | SEQ ID NO. 223 | SEQ ID NO. 224 |
| VH55 | SEQ ID NO. 225 | SEQ ID NO. 226 | SEQ ID NO. 227 |
| VH56 | SEQ ID NO. 228 | SEQ ID NO. 229 | SEQ ID NO. 230 |
| VH57 | SEQ ID NO. 231 | SEQ ID NO. 232 | SEQ ID NO. 233 |
| VH58 | SEQ ID NO. 234 | SEQ ID NO. 235 | SEQ ID NO. 236 |
| VH59 | SEQ ID NO. 237 | SEQ ID NO. 238 | SEQ ID NO. 239 |
| VH60 | SEQ ID NO. 240 | SEQ ID NO. 241 | SEQ ID NO. 242 |
| VH61 | SEQ ID NO. 243 | SEQ ID NO. 244 | SEQ ID NO. 245 |
| VH62 | SEQ ID NO. 246 | SEQ ID NO. 247 | SEQ ID NO. 248 |
| VH63 | SEQ ID NO. 249 | SEQ ID NO. 250 | SEQ ID NO. 251 |
| VH64 | SEQ ID NO. 252 | SEQ ID NO. 253 | SEQ ID NO. 254 |
| VH65 | SEQ ID NO. 255 | SEQ ID NO. 256 | SEQ ID NO. 257 |
| VH66 | SEQ ID NO. 258 | SEQ ID NO. 259 | SEQ ID NO. 260 |

each CDR1, CDR2 and CDR3 is coded according to the prevailing analysis methods of KABAT, Chothia or IMGT; preferably, the substitution is a conservative amino acid substitution.

2. The nanobody or the antigen-binding fragment of claim 1, wherein
(1) the CDR1, CDR2 and CDR3 have sequences as shown in SEQ ID NO. 63, 64 and 65, respectively;
(2) the CDR1, CDR2 and CDR3 have sequences as shown in SEQ ID NO. 66, 67 and 68, respectively;
(3) the CDR1, CDR2 and CDR3 have sequences as shown in SEQ ID NO. 69, 70 and 71, respectively;
(4) the CDR1, CDR2 and CDR3 have sequences as shown in SEQ ID NO. 72, 73 and 74, respectively;
(5) the CDR1, CDR2 and CDR3 have sequences as shown in SEQ ID NO. 75, 76 and 77, respectively;
(6) the CDR1, CDR2 and CDR3 have sequences as shown in SEQ ID NO. 78, 79 and 80, respectively;
(7) the CDR1, CDR2 and CDR3 have sequences as shown in SEQ ID NO. 81, 82 and 83, respectively;
(8) the CDR1, CDR2 and CDR3 have sequences as shown in SEQ ID NO. 84, 85 and 86, respectively;
(9) the CDR1, CDR2 and CDR3 have sequences as shown in SEQ ID NO. 87, 88 and 89, respectively;
(10) the CDR1, CDR2 and CDR3 have sequences as shown in SEQ ID NO. 90, 91 and 92, respectively;
(11) the CDR1, CDR2 and CDR3 have sequences as shown in SEQ ID NO. 93, 94 and 95, respectively;
(12) the CDR1, CDR2 and CDR3 have sequences as shown in SEQ ID NO. 96, 97 and 98, respectively;
(13) the CDR1, CDR2 and CDR3 have sequences as shown in SEQ ID NO. 99, 100 and 101, respectively;
(14) the CDR1, CDR2 and CDR3 have sequences as shown in SEQ ID NO. 102, 103 and 104, respectively;
(15) the CDR1, CDR2 and CDR3 have sequences as shown in SEQ ID NO. 105, 106 and 107, respectively;
(16) the CDR1, CDR2 and CDR3 have sequences as shown in SEQ ID NO. 108, 109 and 110, respectively;
(17) the CDR1, CDR2 and CDR3 have sequences as shown in SEQ ID NO. 111, 112 and 113, respectively;
(18) the CDR1, CDR2 and CDR3 have sequences as shown in SEQ ID NO. 114, 115 and 116, respectively;
(19) the CDR1, CDR2 and CDR3 have sequences as shown in SEQ ID NO. 117, 118 and 119, respectively;
(20) the CDR1, CDR2 and CDR3 have sequences as shown in SEQ ID NO. 120, 121 and 122, respectively;
(21) the CDR1, CDR2 and CDR3 have sequences as shown in SEQ ID NO. 123, 124 and 125, respectively;
(22) the CDR1, CDR2 and CDR3 have sequences as shown in SEQ ID NO. 126, 127 and 128, respectively;
(23) the CDR1, CDR2 and CDR3 have sequences as shown in SEQ ID NO. 129, 130 and 131, respectively;
(24) the CDR1, CDR2 and CDR3 have sequences as shown in SEQ ID NO. 132, 133 and 134, respectively;
(25) the CDR1, CDR2 and CDR3 have sequences as shown in SEQ ID NO. 135, 136 and 137, respectively;
(26) the CDR1, CDR2 and CDR3 have sequences as shown in SEQ ID NO. 138, 139 and 140, respectively;
(27) the CDR1, CDR2 and CDR3 have sequences as shown in SEQ ID NO. 141, 142 and 143, respectively;
(28) the CDR1, CDR2 and CDR3 have sequences as shown in SEQ ID NO. 144, 145 and 146, respectively;
(29) the CDR1, CDR2 and CDR3 have sequences as shown in SEQ ID NO. 147, 148 and 149, respectively;
(30) the CDR1, CDR2 and CDR3 have sequences as shown in SEQ ID NO. 150, 151 and 152, respectively;
(31) the CDR1, CDR2 and CDR3 have sequences as shown in SEQ ID NO. 153, 154 and 155, respectively;
(32) the CDR1, CDR2 and CDR3 have sequences as shown in SEQ ID NO. 156, 157 and 158, respectively;
(33) the CDR1, CDR2 and CDR3 have sequences as shown in SEQ ID NO. 159, 160 and 161, respectively;
(34) the CDR1, CDR2 and CDR3 have sequences as shown in SEQ ID NO. 162, 163 and 164, respectively;
(35) the CDR1, CDR2 and CDR3 have sequences as shown in SEQ ID NO. 165, 166 and 167, respectively;
(36) the CDR1, CDR2 and CDR3 have sequences as shown in SEQ ID NO. 168, 169 and 170, respectively;
(37) the CDR1, CDR2 and CDR3 have sequences as shown in SEQ ID NO. 171, 172 and 173, respectively;
(38) the CDR1, CDR2 and CDR3 have sequences as shown in SEQ ID NO. 174, 175 and 176, respectively;
(39) the CDR1, CDR2 and CDR3 have sequences as shown in SEQ ID NO. 177, 178 and 179, respectively;
(40) the CDR1, CDR2 and CDR3 have sequences as shown in SEQ ID NO. 180, 181 and 182, respectively;
(41) the CDR1, CDR2 and CDR3 have sequences as shown in SEQ ID NO. 183, 184 and 185, respectively;
(42) the CDR1, CDR2 and CDR3 have sequences as shown in SEQ ID NO. 186, 187 and 188, respectively;
(43) the CDR1, CDR2 and CDR3 have sequences as shown in SEQ ID NO. 189, 190 and 191, respectively;
(44) the CDR1, CDR2 and CDR3 have sequences as shown in SEQ ID NO. 192, 193 and 194, respectively;
(45) the CDR1, CDR2 and CDR3 have sequences as shown in SEQ ID NO. 195, 196 and 197, respectively;
(46) the CDR1, CDR2 and CDR3 have sequences as shown in SEQ ID NO. 198, 199 and 200, respectively;
(47) the CDR1, CDR2 and CDR3 have sequences as shown in SEQ ID NO. 201, 202 and 203, respectively;
(48) the CDR1, CDR2 and CDR3 have sequences as shown in SEQ ID NO. 204, 205 and 206, respectively;
(49) the CDR1, CDR2 and CDR3 have sequences as shown in SEQ ID NO. 207, 208 and 209, respectively;
(50) the CDR1, CDR2 and CDR3 have sequences as shown in SEQ ID NO. 210, 211 and 212, respectively;
(51) the CDR1, CDR2 and CDR3 have sequences as shown in SEQ ID NO. 213, 214 and 215, respectively;
(52) the CDR1, CDR2 and CDR3 have sequences as shown in SEQ ID NO. 216, 217 and 218, respectively;
(53) the CDR1, CDR2 and CDR3 have sequences as shown in SEQ ID NO. 219, 220 and 221, respectively;
(54) the CDR1, CDR2 and CDR3 have sequences as shown in SEQ ID NO. 222, 223 and 224, respectively;
(55) the CDR1, CDR2 and CDR3 have sequences as shown in SEQ ID NO. 225, 226 and 227, respectively;
(56) the CDR1, CDR2 and CDR3 have sequences as shown in SEQ ID NO. 228, 229 and 230, respectively;
(57) the CDR1, CDR2 and CDR3 have sequences as shown in SEQ ID NO. 231, 232 and 233, respectively;
(58) the CDR1, CDR2 and CDR3 have sequences as shown in SEQ ID NO. 234, 235 and 236, respectively;
(59) the CDR1, CDR2 and CDR3 have sequences as shown in SEQ ID NO. 237, 238 and 239, respectively;
(60) the CDR1, CDR2 and CDR3 have sequences as shown in SEQ ID NO. 240, 241 and 242, respectively;
(61) the CDR1, CDR2 and CDR3 have sequences as shown in SEQ ID NO. 243, 244 and 245, respectively;
(62) the CDR1, CDR2 and CDR3 have sequences as shown in SEQ ID NO. 246, 247 and 248, respectively;
(63) the CDR1, CDR2 and CDR3 have sequences as shown in SEQ ID NO. 249, 250 and 251, respectively;
(64) the CDR1, CDR2 and CDR3 have sequences as shown in SEQ ID NO. 252, 253 and 254, respectively;
(65) the CDR1, CDR2 and CDR3 have sequences as shown in SEQ ID NO. 255, 256 and 257, respectively;
(66) the CDR1, CDR2 and CDR3 have sequences as shown in SEQ ID NO. 258, 259 and 260, respectively; or,
(67) the CDR1, CDR2 and CDR3 have a sequence combination having 1, 2, 3 or more amino acid insertions, deletions and/or substitutions compared with the above sequence combinations (1)-(66); it is preferably a substitution, more preferably a substitution of a conservative amino acid residue.

3. The nanobody or the antigen-binding fragment of claim 1, wherein the nanobody or the antigen-binding fragment comprises:
(1) a variable region having a sequence as shown in SEQ ID NO: 19, 21, 23, 25, 27, 29, 31, 33, 35, 37, 39, 41, 43, 45, 47, 49, 51, 53, 55, 57, 59 or 61;
(2) an amino acid sequence having at least 90% identity, preferably at least 91%, 92%, 93%, 94%, 95%, 96%, 97%, 98% or 99% identity with the sequence shown in (1) above; or,
(3) the framework region of the nanobody or the antigen-binding fragment has at least 90% identity, preferably at least 91%, 92%, 93%, 94%, 95%, 96%, 97%, 98%, or 99% identity with the framework region of the amino acid sequence as shown in SEQ ID NO: 19, 21, 23, 25, 27, 29, 31, 33, 35, 37, 39, 41, 43, 45, 47, 49, 51, 53, 55, 57, 59 or 61.

4. The nanobody or the antigen-binding fragment of claim 1, wherein the nanobody or the antigen-binding fragment binds to human EGFR and EGFRvIII with a dissociation constant (KD) of no more than 10⁻⁷ nM, and binds to cynomolgus monkey EGFR with a dissociation constant (KD) of no more than 10⁻⁸ nM;
optionally, the nanobody or the antigen-binding fragment binds to or does not bind to monkey EGFR protein;
optionally, the nanobody or the antigen-binding fragment binds to or does not bind to murine EGFR protein;
optionally, the nanobody or the antigen-binding fragment does not compete with antibody C225 or antibody 7D12.

5. The nanobody or the antigen-binding fragment of claim 1, wherein the antibody or the antigen-binding fragment comprises a sequence of the constant region of any one of antibody IgG1, IgG2, IgG3, IgG4, IgA, IgM, IgE or IgD; preferably, comprises a sequence of the constant region of antibody IgG1, IgG2, IgG3 or IgG4.

6. The nanobody or the antigen-binding fragment of claim 1, wherein the antibody or the antigen-binding fragment further comprises an antibody constant region sequence without a CH1 fragment.

7. The nanobody or the antigen-binding fragment of claim 1, wherein the antibody or the antigen-binding fragment further comprises an antibody constant region sequence with CH2 and CH3 fragments, or, the antibody or the antigen-binding fragment further comprises an antibody Fc region;
the antibody constant region or the antibody Fc region is linked to the antibody or the antigen-binding fragment with or without a linker peptide;
optionally, the antibody constant region or the antibody Fc region is derived from camelidae, mice, rats, rabbits, sheep or humans;
optionally, the antibody constant region or the antibody Fc region is derived from IgG, IgA, IgM, IgD or IgE.

8. The nanobody or the antigen-binding fragment of claim 1, wherein the antibody or the antigen-binding fragment is:
(1) a chimeric antibody or a fragment thereof;
(2) a humanized antibody or a fragment thereof; or,
(3) a fully human antibody or a fragment thereof;
preferably, the antibody or the antigen-binding fragment is selected from a monoclonal antibody, a polyclonal antibody, a natural antibody, an engineered antibody, a monospecific antibody, a multispecific antibody (for example, a bispecific antibody), a monovalent antibody, a multivalent antibody, a full-length antibody, an antibody fragment, a naked antibody, a conjugated antibody, a humanized antibody, a fully human antibody, Fab, Fab′, F(ab′)2, Fd, Fv, scFv, a diabody or a single domain antibody.

9. The nanobody or the antigen-binding fragment of claim 1, wherein the nanobody or the antigen-binding fragment is further coupled with a therapeutic agent or a tracer; preferably, the therapeutic agent is selected from a radioisotope, a chemotherapeutic agent or an immunomodulator, and the tracer is selected from a radiological contrast agent, a paramagnetic ion, a metal, a fluorescent label, a chemiluminescence label, an ultrasound contrast agent or a photosensitizer.

10. A multispecific antigen-binding molecule, wherein the multispecific antigen-binding molecule comprises a first antigen-binding module and a second antigen-binding module, the first antigen-binding module comprises the nanobody or the antigen-binding fragment of claim 1, the second antigen-binding module specifically binds to other antigens than EGFR or binds to an EGFR epitope different from the first antigen-binding module;
preferably, the other antigens are selected from CD3, PD-1, PD-L1, Her2, EpCAM, CD16, CD20, CD30, CD33, CD47, CD52, CD64, CD133, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, or FAP;
preferably, the multispecific antibody is a bispecific antibody, a trispecific antibody or a tetraspecific antibody.

11. A chimeric antigen receptor (CAR), wherein the chimeric antigen receptor at least comprises an extracellular antigen-binding domain, a transmembrane domain and an intracellular signaling domain, and the extracellular antigen-binding domain comprises the nanobody or the antigen-binding fragment of claim 1.

12. An immune effector cell, wherein the immune effector cell comprises the chimeric antigen receptor of claim 11 or comprises a nucleic acid fragment encoding the chimeric antigen receptor of claim 11;
preferably, the immune effector cell is selected from a T cell, a NK cell (a natural killer cell), a NKT cell (a natural killer cell), a monocyte, a macrophage, a dendritic cell or a mast cell; the T cell may be selected from an inflammatory T cell, a cytotoxic T cell, a regulatory T cell (Treg) or a helper T cell;
preferably, the immune effector cell is an allogeneic immune effector cell or an autologous immune cell.

13. An isolated nucleic acid molecule, wherein the nucleic acid molecule encodes the nanobody or the antigen-binding fragment of claim 1.

14. An expression vector, wherein the expression vector comprises the isolated nucleic acid molecule of claim 13.

15. An isolated host cell, wherein the isolated host cell comprises the isolated nucleic acid molecule of claim 13; preferably, the host cell is a eukaryotic cell or a prokaryotic cell; more preferably, the host cell is derived from a mammalian cell, a yeast cell, an insect cell, Escherichia coli and/or Bacillus subtilis; more preferably, the host cell is selected from HEK293E cell or CHO cell.

16. A method for preparing the antibody or the antigen-binding fragment of claim 1, wherein a host cell is cultured under appropriate conditions, and the antibody or the antigen-binding fragment is isolated, and
wherein the host cell is an isolated host cell comprising an isolated nucleic acid molecule, and the nucleic acid molecule encodes the nanobody or the antigen-binding fragment.

17. A method for preparing an immune effector cell, wherein the method comprises introducing a nucleic acid fragment encoding the chimeric antigen receptor of claim 11 into the immune effector cell, optionally, the method further comprises enabling the immune effector cell to express the chimeric antigen receptor.

18. A pharmaceutical composition, wherein the composition comprises the antibody or the antigen-binding fragment of claim 1; preferably, the composition further comprises a pharmaceutically acceptable carrier, diluent or adjuvant; preferably, the pharmaceutical composition further comprises an additional antineoplastic agent.

19. (canceled)

20. A method for preventing and/or treating a tumor disease or an inflammatory disease, wherein the method comprises administering an effective amount of the antibody or the antigen-binding fragment of claim 1 to a patient in need thereof;
preferably, the tumor disease or the inflammatory disease is a tumor disease or an inflammatory disease with EGFR overexpression; more preferably, the tumor disease is preferably glioma, melanoma, glioblastoma, sarcoma, brain tumor, non-small cell lung cancer, bladder cancer, breast cancer, endometrial cancer, lung cancer, ovarian cancer, prostate cancer, colon cancer, stomach cancer, liver cancer, kidney cancer, brain cancer, laryngeal cancer, rectal cancer, pancreatic cancer, head and neck cancer, esophageal adenocarcinoma, esophageal squamous cell carcinoma, solid tumors, non-Hodgkin's lymphoma, thyroid cancer, nasopharyngeal carcinoma, esophageal carcinoma, or skin cancers; the inflammatory disease is preferably inflammatory arthritis, serpedo, psoriasis, rheumatoid arthritis, spondylarthropathies, contact dermatitis, delayed hypersensitivity reaction, endometriosis, scar formation, benign prostatic hyperplasia, eczema, dermatitis, nerve inflammation, liver diseases and nephritis, gastrointestinal diseases, inflammatory bowel diseases, Crohn's disease or gastritis.

21. (canceled)

22. A kit, wherein the kit comprises the antibody or the antigen-binding fragment thereof of claim 1; optionally, the kit further comprises an instruction for use.
